(19) United States
(12) Patent Application Publication
(10) Pub. No.: US 2011/0093959 A1
(43) Pub. Date: Apr. 21, 2011

(54) CRYPTOTIS PARVA SCREENING MODEL FOR ANTIEMETIC POTENTIAL

(75) Inventor: Nissar A. Darmani, Pomona, CA (US)

(73) Assignee: WESTERN UNIVERSITY OF HEALTH SCIENCES, Pomona, CA (US)

(21) Appl. No.: 12/865,597

(22) PCT Filed: Jan. 30, 2009
(86) PCT No.: PCT/US2009/032572

§ 371 Date: Nov. 29, 2010

Related U.S. Application Data
(60) Provisional application No. 61/006,739, filed on Jan. 30, 2008.
(60) Provisional application No. 61/058,723, filed on Jun. 4, 2008.

(51) Int. Cl. A01K 67/00; C07D 407/04

(52) U.S. Cl. 800/3; 800/9; 548/253

(57) ABSTRACT

The present invention provides for anrelate to an animal model for characterizing emesis, and for screening and characterizing emetic and antiemetic agents. In several embodiments, the Least Shrew, Cryptotis parva, provides a specific and rapid behavioral animal model to screen concomitantly both the CNS penetration and the antiemetic potential of antiemetic agents to relieve, for example, chemotherapy-induced emesis.

### RELATED PATENT APPLICATIONS

The present invention is related to and claims the benefit of priority of U.S. Patent applications Ser. No. 61/006,739, filed Jan. 30, 2008, and Ser. No. 61/058,723, filed Jun. 4, 2008.

### FEDERAL FUNDING LEGEND

This invention was made with government support under Grant No. R01CA115331 awarded by the National Cancer Institute. The U.S. Government has certain rights in the invention.

### FIELD OF THE INVENTION

The embodiments of the present invention relate to an animal model for characterizing emesis, and for screening and characterizing emetic and antiemetic agents. In several embodiments, the Least Shrew, Cryptotis parva, provides a specific and rapid behavioral animal model to screen concomitantly both the CNS penetration and the antiemetic potential of antiemetic agents to relieve, for example, cisplatin-induced emesis.

### BACKGROUND

Nausea and vomiting are common side effects of chemotherapy treatment for cancer. Once these side effects begin, they can be difficult to control. Vomiting, or emesis, is thought to be controlled by autonomic nerves, which control involuntary bodily functions such as heartbeat and breathing. Various irritants including smells, tastes, anxiety, pain, motion, or digestive chemicals can trigger the vomiting reflex arc in the brain to initiate emesis. Many factors influence whether a person will experience nausea and vomiting. For example, women and people under the age of fifty are more likely to experience nausea and vomiting from chemotherapy. Also, people who are prone to motion sickness or anxiety are more likely to react to chemotherapy with nausea and vomiting.

Additionally, chemotherapy-treated patients often experience two “phases” (clusters or bouts) of emesis. The first phase, called the acute or immediate phase, occurs from an hour or two after treatment up to about a day after treatment. It is followed by a relatively calm, vomiting-free period from one to a few days long, which is then disturbed by the second phase, the “delayed phase”, of vomiting that occurs after the calm period. Emesis adds to the fatigue and distress cancer patients already suffer, causing reluctance to comply with dosing regimens.

Although there are many anti-emetic drugs available, none of them completely block nausea and vomiting due to chemotherapy, especially where the delayed phase is concerned. Because of this, nausea and vomiting side effects remain problematic. Thus, there is a need for further research into the causes of these symptoms, and especially for improved approaches to screening for anti-emetic agents. For example, there is a need for an animal model that better mimics chemotherapy-induced vomiting in people. The current animal models are disadvantageous for many reasons: they are distinct from humans such that only part of the human responses are mimicked, or they require relatively long observation times to be relevant to rapid screening (especially where delayed phase emesis is concerned), or they involve large animals that require large doses of candidate anti-emetic drugs (raising costs of drug development and screening).

Hence, there is a need for an animal model that consistently mimics human emetic responses, is small enough for low cost, high throughput screening, and provides a means for easily observing the effects of emetics and antiemetics. There is also a need for an animal model that consistently mimics human emetic responses, is small enough for low cost, high throughput screening; and for further investigation of antiemetic potential of CysLT₁ receptor antagonists (or inhibitors of enzymes responsible for leukotriene production) against diverse vomiting stimuli, in the laboratory as well as in humans.

### SUMMARY

The present invention provides for a model for screening and characterization of antiemetic agents. More specifically, the North American Least Shrew, Cryptotis parva, provides a specific and rapid behavioral animal model to screen concomitantly both the CNS penetration and the antiemetic potential of antiemetic agents to relieve, for example, cisplatin-induced emesis.

One embodiment of the present invention provides for the use of C. parva as a specific and rapid behavioral model to screen concomitantly both the CNS penetration and the antiemetic potential of tachykinin NK₁ receptor antagonists.

Another embodiment provides a relatively high-throughput method of screening the anti-emetic efficacy of tachykinin NK₁ receptor antagonists, for use as therapeutic agents for treating emesis. This embodiment includes selecting an animal model of emesis, in which the animal species exhibits CNS penetration and emesis in response to tachykinin NK₁ receptor agonists; dividing test animals into two groups; administering a potential tachykinin NK₁ receptor antagonist to one group of animals and its vehicle to another group of animals; administering a tachykinin NK₁ receptor agonist to both groups of animals; and observing and comparing behavioral indices of CNS penetration and emesis in both groups. In a particular embodiment, the animal species is Cryptotis parva.

The present invention also provides for compositions and methods for treating nausea and vomiting. More specifically, an embodiment that the provides for leukotriene antagonists, such as CysLT₁ antagonists and of leukotriene biosynthesis inhibitors, as antiemetic agents.

The present invention also provides for an animal model for the screening and characterization of antiemetic agents. More specifically, Least Shrew, C. parva, provides a specific and rapid behavioral animal model to screen the antiemetic potential of both CysLT₁ antagonists and of leukotriene biosynthesis inhibitors.

One embodiment provides a relatively high-throughput method of screening the antiemetic efficacy of CysLT₁ receptor antagonists for use as therapeutic agents for treating emesis. Another embodiment provides such a screening method for the antiemetic efficacy of leukotriene biosynthesis inhibitors as therapeutic agents for treating emesis.

Another embodiment includes selecting an animal model of emesis, dividing test animals (such as C. parva), into several groups; pretreating groups of test animals with different doses of a CysLT₁-receptor antagonist and then administering a leukotriene (e.g., LC₄ or LD₄) to the pretreated animals, then recording emetic parameters for a suitable period of time, e.g., for the next thirty minutes.

In another embodiment, the antiemetic potential of CysLT₁-receptor antagonists may be evaluated against inflammatory agents or bacterial emetic toxins.

In yet another embodiment, shrews may be pretreated with varying doses of a CysLT₁-receptor antagonist or an inhibitor of enzymes responsible for leukotriene production, either alone or in combination with either one or more other antiemetics before administration of the emetic. Example antiemetics also include a serotonin 3 receptor (5-HT₃) antagonist such as topisetron, a neurokinin 1 (NK₁) receptor antagonist, cannabinoids, or an anti-inflammatory agent such as dexamethasone. Example emetics include the chemotherapeutic agent cisplatin, which may be administered to shrews in a 10 mg/kg dose.

The present invention provides an animal model useful for investigating the antiemetic efficacy of drugs, both against the immediate and delayed phases of cisplatin-induced vomiting for a longer period of time, e.g., up to forty hours.

The present invention also provides for an animal model in which the mechanisms invoked for immediate and delayed phase emesis may be explored. For example, this model shows, for the first time, that Fos expression increases in delayed phase emesis, and that emesis in both phases is sensitive to cannabinoids, mediated by CB₁ receptors.

The present invention also provides an animal model useful for screening for synergy between two or more antiemetic agents. For example, the antiemetic combination of a cannabinoid (e.g., Δ⁹-THC) and a 5-HT₃ antagonist (e.g., tropisetron) exhibited modest synergy in relieving cisplatin-iduced emesis in the Least Shrew. Conversly, there appeared to be no synergistic effects with a cannabinoid (e.g., Δ⁹-THC) and a corticosteroid (e.g., dexamethasone).

### DESCRIPTION OF THE DRAWINGS

FIG. 1 shows the dose-response emetic effects of varying doses of intraperitoneally-administered cisplatin (CIS) on the feeding behavior of the Least Shrew throughout the 47-hour observation period. Feeding is expressed as the mean (±S.E.M.) number of mealworms eaten per hour. a=Significantly different from night 1 and day 2 in the corresponding dose at P<0.05; b=Significantly different from 0 mg/kg during day 1 at P<0.05; c=Significantly different from 0 mg/kg during night 1 at P<0.05; d=Significantly different from 0 mg/kg during day 2. The time scale for the 20 mg/kg dose of CIS was limited to 31-hour observation due to toxicity.

FIG. 2 demonstrates the dose-response developmental profile of total emesis during the immediate and delayed phases of vomiting in the Least Shrew caused by varying doses of intraperitoneally-administered CIS. Emetic episodes are expressed in terms of mean total emesis per hour (±S.E.M.) during the 47-hour observation period. a=The immediate phase is significantly different from the corresponding delayed phase for the cited dose at P<0.05; b=Significantly different from 0 mg/kg control group at corresponding emetic phase at P<0.05. The time scale for the 20 mg/kg dose of CIS was limited to 31-hour observation due to toxicity.

FIG. 3 shows the dose-response developmental profile of wet vomiting in the Least Shrew during the immediate and delayed phases of CIS-induced emesis. Emetic episodes are expressed in terms of mean (±S.E.M) wet vomits per hour during the 47-hour observation period. a=The immediate phase is significantly different from the corresponding delayed phase for the cited dose at P<0.05; b=Significantly different from 0 mg/kg control group at corresponding emetic phase at P<0.05. The time scale for the 20 mg/kg dose of CIS was limited to 31-hour observation due to toxicity.

FIG. 4 shows the dose-response developmental profile of dry vomiting in the Least Shrew during the immediate and delayed phases of CIS-induced emesis. Emetic episodes are expressed in terms of mean (±S.E.M.) dry vomits per hour during the 47-hour observation time. a=The immediate phase is significantly different from the corresponding delayed phase for the cited dose at P<0.05; b=Significantly different from 0 mg/kg control group at corresponding emetic phase at P<0.05. The time scale for the 20 mg/kg dose of CIS was limited to 31-hour observation due to toxicity.

FIG. 5 presents the dose-response developmental profile of abdominal contractions in the Least Shrew caused by varying doses of intraperitoneally administered CIS. The frequency of contractions is expressed in terms of mean (±S.E.M.) contractions per hour during the 47-hour observation period. a=The immediate phase is significantly different from the corresponding delayed phase for the cited dose at P<0.05; b=Significantly different from 0 mg/kg control group at the corresponding emetic phase at P<0.05. The time scale for the 20 mg/kg dose of CIS was limited to 31-hour observation due to toxicity.

FIG. 6 shows the effect of CIS (0 mg/kg and 10 mg/kg, i.p.) on the tissue levels of 5-HT and its major metabolite 5-HIAA in the shrew frontal cortex (graphs A and C, respectively) and brainstem (graphs B and D, respectively) during the peak immediate (2 hour)-phase and delayed (33 hour)-phase of emesis. a=Significantly different from 0 mg/kg control group at the corresponding time at P<0.05; b=Significantly different from the immediate phase at the corresponding dose.

FIG. 7 depicts the effect of CIS (0 mg/kg and 10 mg/kg, i.p.) on the tissue concentrations of 5-HT and its major metabolite 5-HIAA in the shrew duodenum (graphs A and C, respectively) and jejunum (graphs B and D, respectively) during the peak immediate (2 hour)-phase and delayed (33 hour)-phase of emesis. a=Significantly different from 0 mg/kg control group at the corresponding time at P<0.05, b=Significantly different from the immediate phase at the corresponding dose.

FIG. 8 shows the effect of CIS (0 mg/kg and 10 mg/kg, i.p.) on the tissue levels of dopamine and its major metabolite HVA in the shrew frontal cortex (graphs A and C) and brainstem (graphs B and D, respectively) during the peak immediate (2 hour)-phase and delayed (33 hour)-phase of emesis. a=Significantly different from 0 mg/kg control group at the corresponding time at P<0.05, b=Significantly different from the immediate phase at the corresponding dose.

FIG. 9 depicts the effect of CIS (0 mg/kg and 10 mg/kg, i.p.) on dopamine tissue concentration in the shrew duodenum (graph A) and jejunum (graph B) during the peak immediate (2 hour)-phase and delayed (33 hpir)-phase of emesis. a=Significantly different from 0 mg/kg control group at the corresponding time at P<0.05, b=Significantly different from the immediate phase (2 hour) at the corresponding dose.

FIG. 10 shows the effect of CIS (0 mg/kg and 10 mg/kg, i.p.) on the tissue concentrations of substance P in the shrew frontal cortex (graph A), brainstem (graph B), duodenum (graph C) and jejunum (graph D) during the peak immediate (2 hour)-phase and delayed (33 hour)-phase of emesis. a=Significantly different from 0 mg/kg control group at the corresponding time at P<0.05, b=Significantly different from the immediate phase (2 hour) at the corresponding dose.

FIG. 11 reflects the dose-response emetic effects of varying doses of intraperitoneally-administered substance P (SP) (A and B) and the brain penetrating, NK₁ receptor selective agonist GR73632 (C and D), recorded during the 30 min post-injection observation period in the Least Shrew. Graphs A and C depict enhancements in the frequency (mean±S.E.M.) of emesis, whereas B and D show the percentage of shrews vomiting. Asterisks represent significant differences from corresponding vehicle control (0 mg/kg) at P<0.05 (*), P<0.01 (**) and P<0.001 (***).

FIG. 12 demonstrates the ability of varying doses of intraperitoneally administered GR73632 (a brain penetrating, selective NK₁ receptor agonist) to induce dose-dependent increases in the frequency (mean±S.E.M.) of scratching behavior during a 30 minute observation period in the Least Shrew (Graph A). Graphs B and C show the ability of varying doses of two structurally diverse but selective NK₁ receptor antagonists (CP99,994 and L733060) in suppressing the scratching behavior produced by a 5 mg/kg intraperitoneal dose of GR73632. Asterisks represent significant difference from corresponding vehicle control (0 mg/kg) at P<0.05 (*) and P<0.001 (***).

FIG. 13 shows the antiemetic effects of the NK₁ receptor selective antagonist CP99,940 against substance P-induced emesis (A and B) and GR73632-induced emesis (C and D) in the Least Shrew. Graphs E and F show the ability of another NK₁ receptor selective antagonist, L733060, to suppress emesis produced by GR73632. Different groups of shrews received i.p. vehicle (0 mg/kg), or varying doses of CP99,994 (5, 10, or 20 mg/kg) or L733060 (5, 10, or 20 mg/kg), 30 min prior to an emetic dose of either SP (50 mg/kg) or GR73632 (5 mg/kg). Emetic parameters were recorded for 30 min post emetic injection. A, C and E depict attenuations in the frequency (mean±S.E.M.) of emesis, whereas B, D, and F show reductions in the percentage of shrews vomiting. Asterisks represent significant differences from corresponding vehicle control (0 mg/kg) at P<0.01 (**) and P<0.001 (***).

FIG. 14 reflects the lack of effect of either the selective NK₂ receptor antagonist GR159897 (20 mg/kg, i.p.), or the selective NK₃ receptor antagonist SB218795 (20 mg/kg, i.p.), on the ability of a 5 mg/kg intraperitoneal dose of the selective NK₁ receptor agonist GR73632 to produce emesis and scratching behavior. Graph A represents the frequency of emesis (mean±S.E.M.), graph B depicts the percentage of shrews vomiting, and graph C shows the frequency of scratching (mean±S.E.M.).

FIG. 15 demonstrates time-dependent distribution of exogenously administered SP (50 mg/kg, i.p.) in: (A) Brain stem ( [symbol] ) and frontal cortex ( - - - - ), (B) duodenum ( [symbol] ) and jejunum ( - - - - ), and (C) blood serum. Asterisks represent significant differences from corresponding basal level at P<0.05 (*) and P<0.01 (**).

FIG. 16 shows the immunoreactivity of the neuronal marker protein Fos (Fos-IR) in the brain and gut of GR73632 injected shrews and controls. (A) Coronal section of the dorsal vagal complex (DVC) area of a non-vomiting Least Shrew (saline control) stained for Fos-IR. (B) Fos-IR stained coronal section of the DVC in a shrew which vomited after being given 2.5 mg/kg (i.p.) GR73632. The NTS shows a strong induction of Fos-IR, the DMNX exhibits weaker induction, while the AP is devoid of it following either saline or GR72632 injection. Scale bar for (A) and (B)=100 μm. (C) Fos-IR in the myenteric plexus and intestinal wall of a control shrew. Cells in crypts and villi did not produce Fos-IR, but scattered Fos-IR nuclei were found in the myenteric plexus of shrews following either (C) saline, or (D) GR73632 injection. (D) Fos-IR is greatly enhanced in the myenteric plexus (arrowheads) of the GR73632-injected shrew. Scale bar for (C and D)=40 μm. AP, area postrema; Cr, intestinal crypts; DMNX, dorsal motor nucleus of the vagus nerve; MP, intestinal wall layers including myenteric plexus; NTS, nucleus of the solitary tract; V, intestinal villi.

FIG. 17 is the immunohistochemical analysis of immunotoxin lesion. SSP-SAP (1.2 mg/kg, i.p.) was injected to lesion NK₁ receptor-containing cells in the gut. Immunolabeling for NK₁ receptors and substance P (SP) was used to assess the lesion. (A-B) Labeling in the dorsal vagal complex (DVC) of saline-(A/B) or SSP-SAP-injected (A′/B′) shrews appeared normal for both NK₁ receptor (A/A′) and SP (B/B′) (C-D): Relative to saline control (C) labeling in the small intestine showed a distinct and extensive loss of NK₁ receptor (C′) containing cell bodies and fibers in the myenteric plexus, crypts, and villi, although the loss was not complete. SP-containing fibers and cell bodies also showed indications of degeneration (D′), relative to saline-treated control (D). The inset in (B′) shows the presence of Fos-IR within the DVC following i.p. injection of GR73632 and vomiting in a SSP-SAP-injected shrew, demonstrating that the NTS is still functionally responsive to emesis. Scale bars (except inset)=50 μm.

FIG. 18 demonstrates the emetic and scratching dose-response effects of intraperitoneally administered doses of the brain penetrating selective NK₁-receptor agonist GR63762 in normal (0) and in peripheral NK₁-receptor ablated () shrews during the 30 min observation period immediately following NK₁ agonist injection. On day one, large groups of shrews were treated i.p. either with saline (control) or 1.2 mg/kg SSP-saporin (a non-brain penetrant, NK₁ receptor specific immunotoxin) and on day four were challenged with varying doses of GR73632. Graph A shows dose-dependent increases in the frequency of emesis (mean±S.E.M.), whereas graph B depicts the percentage of shrews vomiting. Graph C presents dose-dependent increases in the frequency of (mean±S.E.M.) scratching behavior. Asterisks represent significant differenced from corresponding vehicle control at P<0.01 (**) and P<0.001 (***), and crosses represent significant differences from the corresponding dose in the NK₁ receptor-intact control group (normal, 0) at P<0.05†.

FIG. 19 presents example metabolites of arachidonic acid that can induce emesis in picogram amounts in the Least Shrew. Compounds found experimentally to produce emesis are highlighted light gray (e.g., arachidonic acid, PGG₂, 20 Hydroxy PGE₂, 20 Hydroxy PGE_{2α}, PGF_{2α}, LTC₄, LTD₄) while related non-emetic compounds are black (e.g., PGD₂, PGE₂, PGI₂, LTE₄, LTF₄). The appropriate metabolic systems are dark gray (e.g., LTC₄ Synthase, PGI Synthase, PGF Synthetase/Aldehyde reductase). A number of other arachidonic acid metabolic cascades whose compounds have not been tested are not diagrammed.

FIG. 20 shows the dose-response emetic effects of varying doses of intraperitoneally-administered leukotriene LTC₄ recorded during the 30 min post-injection observation period in the Least Shrew. The graph depicts enhancements in the frequency (mean±S.E.M.) of emesis.

FIG. 21 shows the antiemetic effects of the CysLT₁ receptor selective antagonist pranlucast against Leukotriene LTC₄-induced emesis in the Least Shrew; attenuations in the frequency (mean±S.E.M.) of emesis. Different groups of shrews received i.p. vehicle (0 mg/kg), or varying doses of pranlucast (2.5, 5, or 10 mg/kg) 30 min prior to an emetic dose of lekotriene LTC₄ (1 mg/kg). Emetic parameters were recorded for 30 min post emetic injection.

FIG. 22 shows the antiemetic effects of the CysLT₁ receptor selective antagonist pranlucast against Leukotriene LTC₄-induced emesis in the Least Shrew; reductions in the percentage of shrews vomiting. Dosing and parameters as in FIG. 21.

FIG. 23 depicts both tropisetron (0, 0.025, 0.1, 0.25, 1, and 5 mg/kg) and Δ⁹-THC (0, 0.1, 0.25, 0.5, 1, and 5 mg/kg) by themselves reduced the frequency of CIS-induced (20 mg/kg) emesis in a dose-dependent manner. Only low doses of Δ⁹-THC (0.25 and 0.5 mg/kg) in combination with low doses of tropisetron (0.025 and 0.1 mg/kg) had significantly greater antiemetic efficacy in reducing the frequency of emesis relative to corresponding vehicle-treated tropisetron control groups. *Significantly different from 0 mg/kg Δ⁹-THC control group for the corresponding doses of tropisetron, P<0.05. ▾ Significantly different from the corresponding tropisetron vehicle control group, P<0.05.

FIG. 24 shows tropisetron dose-dependently and completely protected shrews from CIS-induced (20 mg/kg) emesis. Δ⁹-THC significantly reduced the number of shrews vomiting up to 92% in a dose-dependent manner. Δ⁹-THC had no significant synergistic efficacy with tropisetron in reducing the percentage of animal vomiting. *Significantly different from 0 mg/kg Δ⁹-THC control group for the corresponding doses of tropisetron, P<0.05. ▾ Significantly different from the corresponding tropisetron vehicle control group, P<0.05.

FIG. 25 shows dexamethasone (DEX) (0, 0.25, 1, 5, 10, and 20 mg/kg) by itself failed to significantly attenuate the frequency of CIS-induced (20 mg/kg) emesis. Δ⁹-THC by itself significantly reduced the frequency of emesis produced by CIS in a dose-dependent manner. At the doses tested, Δ⁹-THC had no significant additive or synergistic antiemetic activity with dexamethasone. *Significantly different from 0 mg/kg Δ⁹-THC control group for the corresponding dexamethasone doses, P<0.05.

FIG. 26 shows that DEX (0, 0.25, 1, 5, 10, and 20 mg/kg) failed to significantly reduce the number of shrews vomiting in response to CIS (20 mg/kg) exposure. Δ⁹-THC completely protected shrews from CIS-induced emesis in a dose-dependent manner. Δ⁹-THC had no significant additive activity with dexamethasone in reducing the emesis percentage of animals. *Significantly different from 0 mg/kg Δ⁹-THC control group for the corresponding dexamethasone doses P<0.05.

FIG. 27 is bar graphs of counts of Fos-immunoreactive nuclei following acute or delayed phase emesis in the Least Shrew. Fos-IR nuclei in the dorsal vagal complex (DVC) were analyzed for each nuclear region in the DVC. Induction of Fos (and vomiting) was through treatment with 10 mg/kg CIS (i.p.). Means and standard errors of Fos-IR nuclei are graphed for each pre-treatment condition, each subnucleus of the DVC, and both phases of CIV. Significantly reduced numbers of Fos-immunopositive nuclei (*P<0.05) were noted after THC injection when compared to either the vehicle or SR141716A+THC conditions. When regions of the DVC having the same treatment conditions were compared between acute and delayed phases, the AP and NTS had significantly fewer († p<0.05) Fos+nuclei in the delayed phase than in the acute phase of emesis when treated with vehicle or SR141716A+THC, but not when treated with THC alone (P>0.1). Numbers of Fos+nuclei in the DMNX during the delayed phase were not significantly different from the acute phase (P>0.12). Abbreviations: AP—area postrema; DMNX—dorsal motor nucleus of the vagus nerve; NTS—nucleus of the solitary tract.

FIG. 28 presents examples of Fos-IR in the dorsal vagal complex of the Least Shrew following CIS-induced emesis. Sagittal sections through the DVC demonstrate different numbers of Fos-IR nuclei depending on the emetic phase or drug treatment. Vehicle-injected controls (panel A) were effectively devoid of Fos-IR. CIS-injected shrews demonstrated a robust increase in Fos-IR nuclei (black ovals) following the acute phase of emesis (panel B), and a less robust but still significant increase in Fos-IR following the delayed phase (panel C). Injection of THC produces near-control levels of Fos-IR nuclei (panel D), an effect reversible by blockade of CB₁ receptors with SR141716A (panel E). When a transmitted light image of Fos-IR is overlaid on a confocal image of CB₁ receptor-IR from the same microscopic field of the NTS of shrews following acute phase emesis (panel F), Fos-IR nuclei (pseudocolored white ovals) can be seen within unlabeled somata enmeshed in the CB₁ receptor-IR terminal-like structures (grey dots). Asterisks mark the somata of unlabeled (Fos-/CB₁-IR negative) neurons, arrowheads mark CB₁ receptor-IR terminals apposed to NTS somata, and arrows identify somata with both Fos-IR nuclei and CB₁ receptor-IR structures apposed to them. Abbreviations: A—area postrema; DMNX—dorsal motor nucleus of the vagus; NTS—nucleus of the solitary tract. Scale bars=50 μm for A-E; 20 μm for F.

FIG. 29 shows confocal micrographs of cannabinoid receptor, 5-HT, and substance P immunolabeling in the dorsal vagal complex of the least shrew. (A) Sagittal section similar to that in FIG. 28C, stained for CB₁ receptor-IR. CB₁-IR was localized mostly to the NTS. (B) Coronal section of the DVC demonstrating CB₂-IR, which localizes to the brainsurface and choroid plexus (arrows), and to non-neuronal elements within the brainstem that appear vascular in nature (arrowheads). Elements suggestive of neuronal origin were absent. (C) Multiple-labeling of 5-HT, CB₁ receptor, and SP in the NTS of the shrew demonstrated a variety of interactions. Asterisks demonstrate unlabeled somata apposed to punctate (terminal-like) immunolabeling for 5-HT, CB₁ receptors, and/or SP. Although many terminal-like structures were singly labeled, some showed colocalization of 5-HT/CB₁ receptor (arrows), SP/CB₁ receptor (arrowheads), or 5-HT/SP (double arrow). A single terminal also demonstrated colocalization of all three antigens (double arrowhead). (D) Zero-primary coronal DVC section using the anti-rat secondary antibody and Alexa594-conjugated tyramide amplification. (E) Zero-primary coronal DVC section using the anti-rabbit secondary and Alexa488-conjugated tyramide. Abbreviations: 12 hypoglossal nucleus; 4V 4th ventricle; AP area postrema; ChP—choroid plexus; Cu—cuneate nucleus; mlf—medial longitudinal fasciculus; NTS—nucleus of the solitary tract. Scale bars—(A), 50 μm; (B), 500 μm; (C), 10 μm; (D-E), 50 μm.

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

The present invention provides for an animal model that better mimics chemotherapy-induced vomiting in humans. The current animal models are disadvantageous for many reasons: they are distinct from humans such that only part of the human responses are mimicked, or they require relatively long observation times to be valid (especially where delayed phase emesis is concerned), or they involve large animals that require large doses of candidate antiemetic drugs (raising costs of drug development and screening).

An animal model, C. parva, was introduced recently as a versatile model for studying emesis. Darmani,105 J. Neurol. Transm 1143-54 (1998); Darmani et al., 106 J. Neurbiol. Transm. 1045-61 (1999); Darmani, 24 Neuropsychopharmacol. 198-203 (2001a)), and could potentially be utilized as a rapid and specific behavioral model for the study of both CNS penetration and the antiemetic potential of agents such as NK₁ receptor antagonists. C. parva are tiny carnivorous mammals, native to the eastern half of the north and central Americas. Unlike other shrews, C. parva are social, burrowing and nesting in groups, and even sharing food. This characteristic has enabled adaptation to laboratory colonies. In the wild, Least Shrews eat a wide variety of invertebrates, including insects, earthworms, snails, and slugs. Plant material makes up a tiny portion of the shrew diet. In captivity, shrews may be fed a blend of cat food, mouse chow, meal worms, and the like. Additionally, C. parva breed well in captivity. Specimens are available through the Western University of Health Sciences.

Although the neurotransmitter basis of chemotherapy-induced vomiting (CIV) is thought to be multifactorial, it is generally accepted that acute (immediate) CIV is mainly due to the release of serotonin (5-HT) within the gastrointestinal tract, and the delayed phase occurs following substance P (SP) release in the brainstem. The present invention provides for a model to test this dogma. The temporal development of CIS's immediate and delayed emetic effects in the Least Shrew showed concomitant changes in the turnover of major emetic neurotransmitters both in the central and peripheral loci associated with CIV. CIS caused dose- and time-dependent emetic effects. More specifically, a 10 mg/kg dose of CIS produced both phases of emesis with corresponding peak mean frequencies occurring at 2 hours to 3 hours and 33 hours post-treatment. These CIS(10 mg/kg, i.p.)-induced peak immediate and delayed phases were associated with concomitant increases in the turnover of 5-HT, dopamine and SP, in both the brainstem and jejunum. The increases during both phases appear to be site-specific because neurotransmitter release was not altered persistently in the shrew frontal cortex or duodenum, although increases or decreases did occur occasionally. These findings suggest that the Least Shrew appears to be a sensitive and rapid emesis model for both phases of CIV, and both emetic phases are associated with specific increases in the release of all of the cited neurotransmitters in both the brainstem and jejunum. Thus, the generally accepted neurotransmitter dogma needs to be updated because more recent neurochemical studies in humans as well as other clinical findings support the current basic results obtained in the Least Shrew.

Additionally, there is significant evidence, in both animal studies and clinical trials, that the tachykinin substance P (SP) is thought to play a cardinal role in emesis, via the activation of central tachykinin NK₁ receptors. The current NK₁-blocking anti-emetic drugs have been slow to develop, however, and only one (aprepitant) is approved for general use thus far. A potential complication to targeting NK₁ is that NK₁ receptors are found in the gastrointestinal tract and enteric nervous system, so the anti-emetic effect may be a peripheral as well as a central effect. In fact, no study has yet confirmed the peripheral or central (or mixed) location of anti-emetic action by NK₁ antagonist drugs. Although part of the delay is related to the development costs of the drugs, there is another complication as well: the current anti-NK₁ drugs are less effective in the acute phase than the more-common anti-serotonin 5-HT₃ receptor antagonists (like ondansetron). Where the current anti-NK₁ drugs shine is in the delayed phase, where they exhibit good anti-emetic effectiveness in clinical trials. Because a good animal model for testing the delayed phase quickly and with minimal expense is lacking, this potential has yet to be exploited fully. The lack of a good animal model also stymies research on whether NK₁ antagonists must penetrate the brain (i.e., central action) to be effective anti-emetics, or whether the drug need only access the gut (i.e., peripheral action). Knowing this, and having an animal model that can differentiate the two conditions, would significantly speed drug discovery and potentially lower the expense (assuming no requirement for central receptor delivery). The present invention provides for an animal model that may be observed visually to characterize the difference in brain-penetrating and non-penetrating tachykinin NK₁ receptor antagonists.

The present invention also provides for methods of treating emesis with leukotriene antagonists, including both CysLT₁ antagonists and inhibitors of leukotriene synthesis, that have demonstrated antiemetic potential and possible clinical utility against emesis produced by a variety of agents including chemotherapeutics, inflammatory agents as well as bacterial toxins and other gastrointestinal conditions.

Additionally, cannabinoids can inhibit the CIV acute phase, albeit through a poorly understood mechanism. The present invention provides for an animal model that facilitates examining the substrates of cannabinoid-mediated inhibition of both the emetic phases via immunolabeling for serotonin, Substance P, cannabinoid receptors 1 and 2 (CB₁, CB₂), and the neuronal activation marker Fos. Briefly, Least Shrews were injected with CIS and one of vehicle, Δ⁹-THC, or both Δ⁹-THC and the CB₁ receptor antagonist SR141716A, and monitored for vomiting. Δ⁹-THC-pretreatment caused concurrent decreases in the number of shrews expressing vomiting and Fos-immunoreactivity (Fos-IR), effects which were blocked by SR141716A-pretreatment. Acute phase vomiting induced Fos-IR in the solitary tract nucleus (NTS), dorsal motor nucleus of the vagus (DMNX), and area postrema (AP), whereas in the delayed phase Fos-IR was not induced in the AP at all, and was induced at lower levels in the other nuclei when compared to the acute phase. CB₁ receptor-IR in the NTS was dense, punctate labeling indicative of presynaptic elements, which surrounded Fos-expressing NTS neurons. CB₂ receptor-IR was not found in neuronal elements, but in vascular-appearing structures. All areas correlated with serotonin- and Substance P-IR. These results support published acute phase data in other species, and are the first describing Fos-IR following delayed phase emesis. The data suggest overlapping but separate mechanisms are invoked for each phase, which are sensitive to antiemetic effects of Δ⁹-THC mediated by CB₁ receptors.

CIV involves both central and peripheral mechanisms (Andrews & Rudd, 164 Handbook Exp. Pharma. 359-440 (2004); Hesketh et al., 39 Eur. J. Cancer 1074-80 (2003); Minami et al., in SEROTONIN & SCI. BASIS ANTIEMETIC THER. 68-76 (Oxford Clin. Commun., Oxford, 1995); Veyratt-Follet et al., 53 Drugs 206-34 (1997). The medullary dorsal vagal complex (DVC) in the brainstem, including the nucleus tractus solitarius (NTS), the dorsal motor nucleus of the vagus (DMNX), and the area postrema (AP) are involved in the central mediation of emesis. Emetic afferents to the DVC arise from diverse brain nuclei, and from peripheral structures such as the gastrointestinal tract (GIT) mainly via the vagus nerve. The AP in the chemoreceptive trigger zone allows bloodborne chemicals absorbed by or secreted from the intestinal mucosa to bypass the blood-brain-barrier. The DMNX receives afferents from vagal nodose ganglion neurons, and sends efferents to the enteric nervous system (ENS), as well as to a central pattern generator (CPG) postulated to be dorsomedial to the nucleus ambiguus and retrofacial nucleus which coordinates peristaltic activity and its reversal during emesis (Hornby, S8A Am. J. Med. 106S-12S (2001); Onishi et al.,136 Auton. Neurosci. 20-30 (2007)). The NTS is a point of convergence and a major integrative site which receives information from diverse afferent inputs including vestibular nuclei, vagal afferents, afferents from several parts of the forebrain (e.g., neocortex and hypothalamus), and the other DVC nuclei. Emetic afferents arise from diverse areas including the AP and from the gastrointestinal tract (GIT) via the vagus nerve.

Although the neurochemical circuits for CIV are poorly understood, several emetic stimuli such as dopamine, serotonin, substance P (SP) and prostaglandins are postulated to contribute to its genesis (Andrews & Rudd, 2004; Minami et al., 99 Pharmacol. Therap. 149-65 (2003); Veyrat-Follet et al., 53 Drugs 206-34 (1997)). The chemotherapeutic agent CIS produces vomiting biphasically in both humans (Hesketh et al., 39 Eur. J. Cancer 1074-80 (2003)), and other emetic species (Jordan et al., 61 Crit. Rev. Oncol. Hematol. 162-175 (2007)); Rudd et al., 119 J. Pharmacol. 931-36 (1996); Rudd et al., 391 Eur. J. Pharmacol. 145-50 (2000); Sam et al., 472 Eur. J. Pharmacol. 135-45 (2003); Tanihata et al., 461 Eur. J. Pharmacol. 197-206 (2003)). In patients, the acute (immediate) emetic phase is comprised of episodes occurring within 24 hour of CIS exposure and the delayed phase between days 2 and 7 post-infusion. The current antiemetic therapy dogma is based upon the premise that in the acute phase CIS releases serotonin from enterochromaffin cells of the GIT, which then stimulates 5-HT₃ receptors on vagal afferents to initiate the vomiting reflex, while the delayed phase emesis is thought to be due to activation of neurokinin NK₁ receptors subsequent to the release of SP in the brainstem (Andrews & Rudd, 2004; Sanger & Andrews, 129 Auton. Neurosci. 3-16 (2006)). Indeed, in cancer patients 5-HT₃ receptor antagonists are effective during the acute phase, whereas NK₁ receptor antagonists improve the antiemetic efficacy of conventional antiemetics during the delayed phase, while inclusion of dexamethasone (an inhibitor of prostaglandin synthesis) in the antiemetic cocktails improves the efficacy of both classes of antiemetics (Hesketh et al., 2003).

The role of dopamine D2 receptors in CIV and utilization of its corresponding antagonists are thought to be of historical interest because these agents were the mainstay of antiemetic therapy prior to the 1980s (Jordan et al., 2007; Veyrat-Follet et al., 53 Drugs 206-34 (1997)). Although these findings are important breakthroughs in clinical oncology, the incidence of nausea and vomiting still remains unacceptably high, and is a major factor in premature discontinuation of chemotherapy. The inability to develop more effective antiemetic regimens reflects the partial appreciation of the relative contribution of multiple emetic neurotransmitters and their temporal interplay in the regulation of both phases of CIV both in the brainstem and the GIT. Indeed, unlike older clinical reports which implicated one neurotransmitter per emetic phase (Cubeddu et al., 66 Br. J. Cancer 198-203 (1992); Janes et al., 34 Eur. J. Cancer 196-98 (1998); Wilder-Smith et al., 72 Cancer 2239-41 (1993)), more recent human studies show a more complex, differential and overlapping involvement of several neurotransmitters in the human blood or urine (Higa et al., 12 Oncol. Pharm. Pract. 109-201 (2006)), during the full time course of CIV. Clinical evidence is supportive of this notion since no single antiemetic is completely effective at blocking emesis in either phase, but when administered together, the antiemetic efficacy of the combination is greater than that of each agent given individually (Hesketh et al., 2003).

Although scant evidence exists on the effect of CIS on the concentrations of dopamine or SP in human plasma or urine, no data exists on the tissue levels of these neurotransmitters in any emetic loci (Higa et al., 2006; Inoue et al., 33 Gan to Kagaku Ryoho 931-36 (2006)). Moreover, although numerous investigators have determined the effects of CIS on 5-HT turnover at a specific phase or during both the immediate and delayed emetic phases either in the brainstem (Endo et al., 7 Biog. Amines 525-33 (1990b), Endo et al., 9 Biog. Amines 163-75 (1992); Liu et al., Re. Commn Mol. Pathol. Phramacol. 97-113 (2003)), blood (Barns et al., 62 Br. J. Cancer, 862-64 (1990); Castejon et al., 291 J. Pharmacol. Exp. Therap. 960-66 (1999)), GIT (Endo et al., 15 j. Toxicol. Sci. 235-44 (1990a); Fukunaka et al., 31 Gen. Pharmacol. 775-81 (1998)), or in the urine (Cubeddu et al., 66 Br. J. Cancer 198-202 (1992); du Bois et al., 5 Support Care Cancer 212-18 (1997); Higa et al., 12 J. Oncol. Pharm. Pract. 109-201 (2006); Janes et al., 34 Eur. J. Cancer 196-98 (1998)), no published study explains the concomitant alterations in tissue levels of one or more of these emetic neurotransmitters simultaneously in the brainstem and GIT during both phases of emesis in a vomiting species. Because CIS can induce both phases of emesis in animals when administered either via the central or peripheral route (Smith et al., 40 J. Pharm. Pharmacol. 142-43 (1988); Tanihata et al., 2003); and both central (Gidda et al., 1995; Higgins et al., 1989; Smith et al., 1988; Yoshida et al., 1992) and peripheral (Fukui et al., 1992; Gidda et al., 273 J. Pharmacol. Exp. Ther. 695-701 (1991); Mutoh et al., 58 Jpn. J. Pharmacol. 321-24 (1992)), loci have already been implicated for serotonin, and more recently for SP's emetic actions (Darmani et al., 1214 Brain Res. 58-72 (2008)), it is imperative to simultaneously evaluate the effects of CIS on the turnover of the discussed emetic neurotransmitters both in the brainstem and the GIT.

To further test the CIV neurotransmitter dogma, the current work establishes the dose-reponse and time-response emetic effects of CIS in the Least Shrew (C. parva) to identify CIS's peak vomiting episodes during the immediate and delayed phases. This emesis model appears to posses emetic responses similar to those of humans (Darmani, 105 Neural. Transm. 1143-54 (1998)). The present invention also provides for the determination of concomitant changes in the tissue levels of the key emetic neurotransmitters (5-HT, dopamine, and SP) and some of their major metabolites (5-HIAA and HVA, respectively, for 5-HT and dopamine) as a measure of their turnover in both the CNS (frontal cortex (as control) and brainstem) and GIT (duodenum and jejunum), during the peak immediate- and delayed phases of vomiting, in response to an effective dose of CIS.

One of the embodiments of the present invention provides for the temporal development of immediate- and delayed-phases of CIS-induced emesis in this species. CIS (i.p.) produced both phases of CIV in a dose-dependent and time-dependent fashion in a manner similar to other emesis models (Milano et al., 274 J. Pharmal. Exp. Ther. 951-61 (1995); Rudd et al., 119 Br. J. Pharacol. 931-36 (1996); Rudd et al., 391 Eur. J. Pharmacol. 145-50 (2000); Sam et al., 472 Eur. J. Pharmacol. 135-45 (2003); Tanihata et al., 2003). The emetic parameters herein are presented per hour as the mean frequencies of total-, wet- and dry-vomits, as well as the mean number of abdominal contractions. The motor component of emesis often consists of both retching and vomiting (Andrews et al., 307 Eur. J. Pharmacol. 305-13 (1996)).

Retching events consist of abdominal contractions accompanied by decreases in thoracic pressure coincident with increases in abdominal pressure. In an emetic event both thoracic and abdominal pressures become positive and this is associated with the expulsion of gastrointestinal content. Several retches occur as a burst that either terminate in gastric expulsion (a vomit event) or does not (a retching event). Visual observation of emesis in the larger species of shrews (Suncus murinus) has revealed that although vomiting can be accurately quantified by direct observation as in other species (Andrews et al., 1996), abdominal and thoracic movements characteristic of retching occur at a frequency too high for individual retches (lasting 250 msec) to be counted, and thus electrophysiological methods were employed to analyze the details of this behavior.

Those investigators who have used direct visual observation report a sequence of retches (four to nine retches) as a retching event (McCarthy & Borison, 266 Am. J. Physiol. 738-43 (1974); Lang, in MECH. & CONTROL EMESIS 71-82 (Bianchi et al., eds. John Libbey Eurotxt Cooloque INSERM, 1992). In this particular study, the observation cage was situated below the camera and each shrew was observed from the dorsal view. CIS caused squeezing contractile movements of the hind portion of the Least Shrew involving contractions of abdominal, lateral and back muscles from caudal to cranial direction lasting 2 to 3 seconds. Because the details of these contractions were not the studied intensely, each contraction might consist of a single retch or train of retches. Least Shrews tended to eject the gastrointestinal content (wet vomiting) following rhythmical undulating contractions of their abdomen, thorax and neck muscles with characteristic up and down and forward shaking of the head with mouth openings and sometimes accompanied by lateral wiping of their mouth on the cage floor. Dry vomits constituted all of the latter behaviors but no ejection of solid or liquid material occurred probably due to an empty stomach.

From statistical and visual considerations, the mean total vomit per hour (wet plus dry vomits), appears to provide the best illustration of the temporal development of both emetic phases. Some shrews in the vehicle-treated group tended to vomit with relatively low frequencies at various times during the 47-hour observation period which were mainly of dry mouth openings. This may be due to the very rapid swallowing of mealworms. The 5 mg/kg dose of CIS tended to increase the mean frequency of total vomiting but significant effects for both phases occurred at its 10 mg/kg dose. Indeed, its peak mean frequency for the immediate phase occurred between the second and third hour of exposure with smaller frequencies occurring up to 13 hours. A quiescent period of practically no emesis occurred up to 24 hours. Thereafter, the frequency increased, reaching maximum at 33 hours, which then declined slowly to basal levels by 47 hours. Moreover, during the immediate phase (10 mg/kg) the vomit frequency was significantly greater than its corresponding vehicle-treated control. It was significantly less, however, relative to the delayed phase vomit frequency, which itself was greater than its corresponding control. Although the peak immediate (third hour) and delayed (43 hours to 45 hours) phases for the 5 mg/kg dose occurred relatively later, the corresponding peaks (between 1 hour and 2 hours, and 23 hours and 25 hours, respectively) for the 20 mg/kg group occurred earlier. The pattern of development of wet and dry vomits followed essentially that of total vomits. Wet vomits generally accounted for the major portion of total emesis for the first couple of hours of CIS exposure during the immediate phase, whereas dry vomits mainly contributed to the remaining episodes during both early and delayed phases. Lower doses of CIS (5 and 10 mg/kg) tended to stimulate eating during the early part of exposure (night 1 and day 1, respectively). On the other hand, the 20 mg/kg CIS dose suppressed feeding after the first hour throughout the observation period, while its 10 mg/kg dose reduced feeding in the delayed phase. CIS also induces such anorexic effects in other species (Takeda et al., 2008).

A closer inspection of the current data and published studies in other emesis models suggests that the details of temporal development of CIS-induced emetic behaviors are somewhat variable, and are probably dependent upon the: (1) CIS dose used, (2) route of administration employed, (3) presentation of emetic parameters either as a single or combinations of behaviors, and (4) possible species differences in CIS action and disposition. Indeed, as with humans (Hesketh et al., 2003), CIS infused in piglets results in immediate and delayed peaks occurred at 2 hour and 22 hour, respectively, with emetic events lasting up to 58 hours (Milano et al., 1995). In cats, CIS induces maximum emetic behaviors between the third and fourth hours for the immediate phase, succeeded by a quiescent period up to 22 hours and then followed by a series of delayed episodes between 22 hours and 38 hours (Rudd et al., 2000). Higher doses of CIS induces both phases in the cat quickly, but toxicity restricted the full observation time. In pigeons, CIS causes immediate and delayed peak behaviors respectively at 2 hours to 4 hours, and 16 hours to 22 hours post-CIS-treatment with lower frequencies being present throughout the 48 hour observation period during which some pigeons died of toxicity (Tanihata et al., 2003). In the ferret, i.p. administered CIS produces the immediate and delayed peaks (3 hours to 4 hours, and 52 hours to 56 hours) later, and higher doses produce the emetic effects earlier, but are toxic (Rudd et al., 1996). The house musk shrew (S. murinus) appears rather insensitive to CIS's emetic effects: only 80% of the animals vomit in response to the largest tested dose of CIS, and toxicity causes fatality in some of these animals and thus limit full observation (Sam et al., 2003).

Abdominal contractions appear to be a normal behavioral repertoire of the Least Shrew ranging from none to very low frequencies per hour and its profile appears to follow the intensity of feeding behavior. CIS also increased the frequency of this behavior in a dose- and time-dependent manner with broad peaks occurring between 3 hours to 7 hours, and 26 hours to 46 hours post-treatment. If these contractions in the Least Shrew represent the retching behavior in other species, then this behavior could be also used to study the antiemetic potential of drugs. Although the mean frequency of these contractions appears to be variable from one experiment to another in the same laboratory (present work vs. Darmani, 1998), such variation also occurs in other species (e.g., Sam et al., 2003 vs. Yamamoto et al., 2004). Overall, the Least Shrew appears to be a robust animal model to investigate the antiemetic potential of drugs against both phases of vomiting caused by a 10 mg/kg intraperitoneal dose of CIS.

Regarding the neurotransmitter basis of CIV, although there is significant neurochemical, electrophysiological and behavioral evidence in support of a major peripheral role for gastrointestinal 5-HT in the mediation of the acute phase, there is additional evidence suggesting involvement of central serotonergic mechanism(s) in this phase, as well as the involvement of 5-HT in the delayed phase. For example: (a) central administration of peripherally-ineffective doses of CIS produce both emetic phases in the pigeon and feline models (Smith et al., 40 J. Pharm. Pharmacol. 142-43 (1988); Tanihata et al., 2003); (b) systemic administration of CIS during the acute phase not only increases indices of 5-HT turnover and/or the activity of its rate limiting synthesis enzyme in the ferret brainstem (Endo et al., 1990a, Minami et al., 1995), and ileum (Kudo et al., 2001), or human plasma (Cubeddu et al., 1992) during the acute phase; but this chemotherapeutic agent also increases 5-HT turnover in the latter loci during the delayed phase (Endo et al., 1990a; Fukunaka et al., 1998; Higa et al., 2006; Liu et al., 2003); (c) though peripheral injection of quaternary forms of 5-HT₃ receptor antagonists (which are unable to pass the blood brain barrier) can prevent emesis produced via systemically administered CIS in the ferret (Gidda et al., 1991), such antagonists potently abolish the induced emesis when injected centrally but not when administered systemically in dogs (Gidda et al., 1995); and (d) the peripherally acting quaternary analog of serotonin, 5-HTQ, has ten-times greater affinity for the 5-HT₃ site, but was three-times less potent than 5-HT in causing emesis when administered systemically in the Least Shrew (Darmani, 1998).

To evaluate the basis of central/peripheral components of CIV neurotransmitter dogma, the brainstem tissue containing the DVC emetic nuclei, the frontal cortex which is not directly involved in emesis, as well as duodenal and jejunal tissues, were chosen to simultaneously measure release and/or turnover in the concentrations of 5-HT, dopamine and SP, and some of their major metabolites. The results presented herein show selective and concurrent changes in 5-HT turnover in specific central and peripheral emetic loci, and support the results of the above discussed serotonergic studies which were published in separate manuscripts by different investigators either during early or delayed CIS exposure, in brainstem or small intestine of other vomiting species. Indeed, CIS failed to significantly affect the basal tissue levels of 5-HT, its major metabolite 5-HIAA, or its turnover (5-HIAA/5-HT ratio) either at the peak acute (i.e., 2 hour post-treatment) or delayed phase (i.e., 33 hour post-exposure) in both shrew frontal cortex and duodenal tissues. CIS significantly increased brainstem 5-HT turnover, however, during both immediate and delayed phases of emesis because the tissue concentrations of 5-HT and 5-HIAA were increased relative to corresponding controls.

Moreover, during the acute phase a similar increase occurred in the jejunal turnover of serotonin, although the tissue concentration of 5-HT was decreased but its metabolite level was significantly increased probably due to rapid 5-HT release and metabolism. Likewise, in the delayed phase, jejunal 5-HT turnover was increased since its tissue concentration was nearly 50% greater than its corresponding control. The discrepancy between these basic studies and early clinical findings which had indicated significant changes in plasma or urinary 5-HT turnover only occurs during the immediate CIV phase is more apparent than real because: (1) some of these studies were either confined to less than the first day of chemotherapy exposure (Barns et al., 62 Br. J. Cancer 862-64 (1990); Castejon et al., 291 J. Pharmacol. Exp. Therap. 960-66 (1999); Cubeddu, 53(S1) Oncol. 18-25 (1996)); or (2) 5-HIAA samples were analyzed at relatively long intervals during the delayed phase which could have masked the observed changes. Indeed, in two of the latter studies, smaller 5-HIAA peaks post-day-1 exposure were observed but ignored, while Higa et al. (2006) concluded a biochemical link between urinary changes in 5-HIAA concentrations with the acute phase emesis in cancer patients which extended to the delayed phase. Thus, the current findings support the view that emesis-related increases in 5-HT turnover occur simultaneously in both brainstem and specific portions of small intestine, and these changes are not only confined to the acute phase but extend to the delayed phase. This provides a plausible explanation that although 5-HT3 receptor antagonists are not very effective by themselves during the delayed phase, they can provide beneficial effect when combined with other antiemetics (Campos et al., 19 J. Clin. Oncol. 1759-67 (2001); Hesketh et al., 2003).

Although shrew brainstem dopamine concentrations tended to increase (PN 0.05) during both phases of vomiting, CIS only significantly raised dopamine tissue levels in the frontal cortex during the immediate phase. Dopamine turnover in the brainstem appears to be increased, however, during both phases since the tissue concentrations of its major metabolite, HVA, were significantly raised by CIS. While duodenal dopamine levels were unaffected, the jejunal tissue concentrations were raised by 171% (P<0.05) during the delayed phase, but the increase in the immediate phase failed to achieve significance. Thus, the role of dopamine in CIV exhibited herein provides direct evidence for increased dopamine turnover in both the brainstem and jejunal tissues associated with both phases of CIV. Indeed, the effect of CIS on increased dopamine function has only been reported either in a PC12 cell line or in the plasma of cancer patients receiving chemotherapy (Inoue et al., 2006; Kasabdji et al., 59 Life Sci. 1793-1802 (1996)). These results support the early clinical observations that dopamine D2 receptor antagonists may have a protective role in the control of CIV (Jordan et al., 61 Crit. Rev. Oncol. Hematol. 162-75 (2007)).

One of the important findings of the present study is the effect of CIS on the release of SP in CIV. Numerous indirect histochemical, electrophysiological, and pharmacological studies have implicated SP and activation of its NK₁ receptor in the brainstem emetic nuclei in the mediation of delayed CIV (Andrews & Rudd, 2004). Until the present invention, there was no direct evidence whether SP produces emesis via NK₁ receptor activation, or whether CIS can potentiate SP release in the brainstem or any other emetic loci. Indeed, in the present invention shows that both SP and its brain-penetrating selective NK₁ receptor agonist, GR73632, induce vomiting via activation of NK₁- (but not NK₂- or NK₃-) receptors located in the nucleus tractus solitarius and the dorsal motor nucleus of the vagus, while gastrointestinal NK₁ receptors play a facilitatory role in the induced emesis.

The evidence presented herein shows that CIS causes tremendous increases (1396% and 956%, respectively) in the brainstem SP tissue levels during both the immediate and delayed phase CIV, the increase being maximal in the initial phase. Moreover, a similar profile but relatively more limited increases (333% and 226.5%, respectively) in jejunal SP tissue concentrations were also observed during both phases of CIV. A recent clinical study supports these findings, because changes in the plasma SP concentration of patients receiving high dose CIS had a similar profile of increases in both acute and delayed phase CIV (Higa et al., 2006). The observed changes in the current study seem to be region specific since the frontal cortex SP concentration during delayed phase was decreased, while duodenal concentrations were unaffected in either phase. These findings fit well with other clinical observations that inclusion of NK₁ receptor antagonists in antiemetic cocktails improves their overall efficacy during both phases of CIV (Campos et al., 2001; Hesketh et al., 2003).

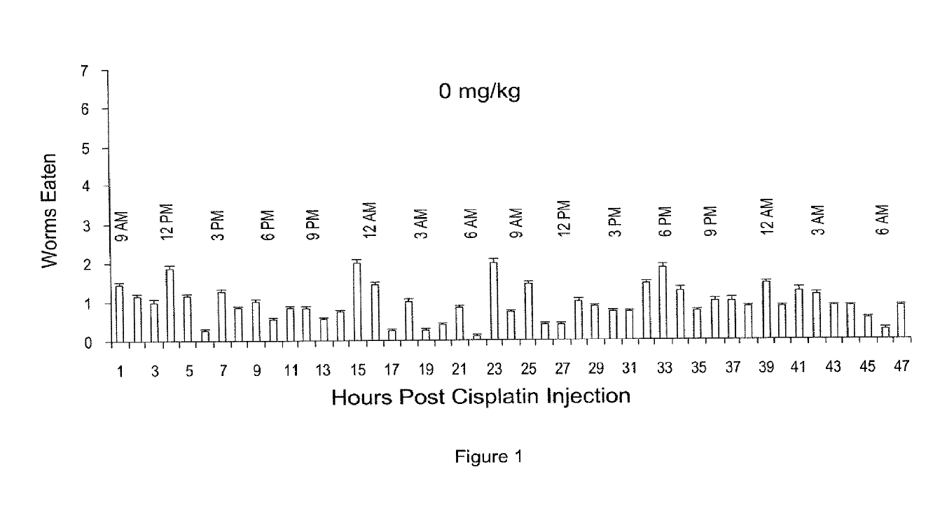

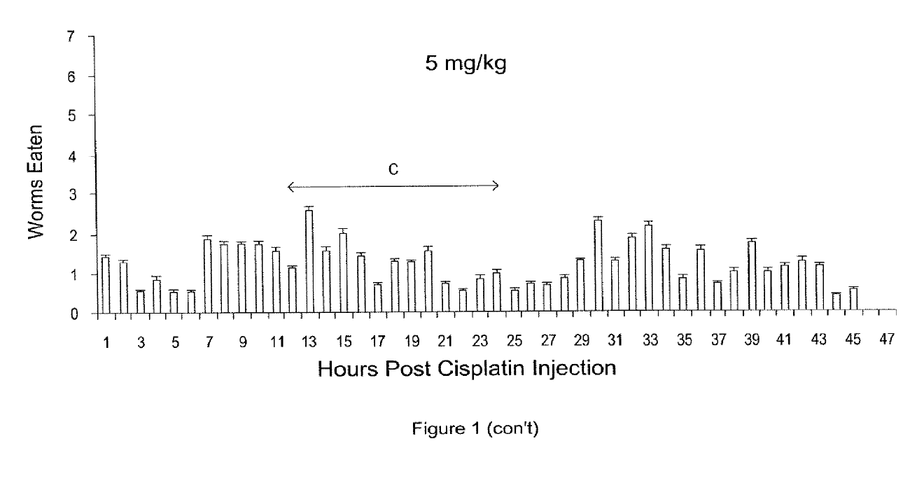

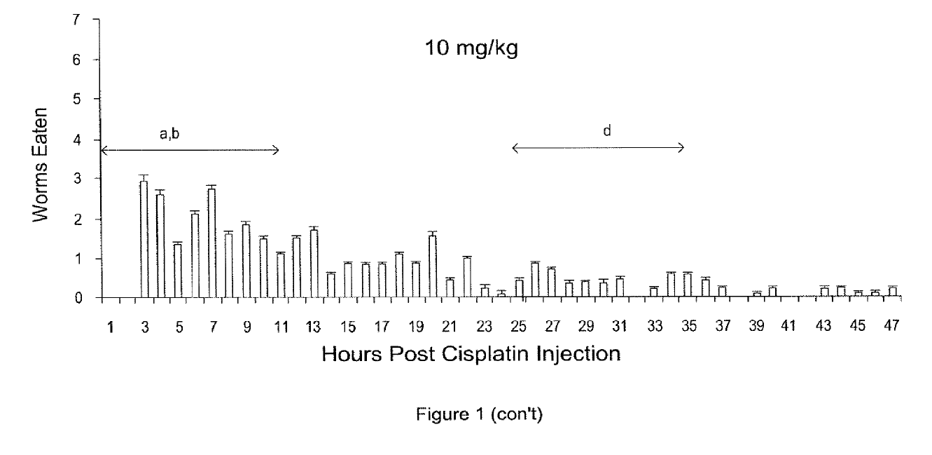

In particular, vehicle-treated shrews tended to continually eat approximately 1 to 1.5 mealworms per hour throughout most of the observation period (FIG. 1). They tended to eat less (0.25 to 1 mealworms per hour) for a few hours after 12 PM. For statistical purposes the eating pattern (number of mealworms eaten per hour) was divided into four periods: day 1 (0 hour to 11 hour post-CIS injection), night 1 (12 hour to 24 hour post-CIS injection), day 2 (25 hours to 35 hours post-CIS injection) and night 2 (36 hours to 47 hours post-CIS injection). Because shrews tended to die from the 20 mg/kg CIS treatment during night 2 of the observation period, the eating data were only analyzed for day 1, night 1 and day 2 for the 0, 5, 10 and 20 mg/kg treatment groups.

A two-way repeated measure of analysis of variance indicated a significant overall effect for time (day 1, night 1, and day 2) and CIS dose (0, 5, 10, and 20 mg/kg) as factors [F (6, 54)=4.90, P<0.002] (FIG. 1). In addition, significant main effect differences were observed in the number of worms eaten during the cited time periods in the 10 mg/kg [F (2, 14)=9.67, P<0.01] and 20 mg/kg [F (2, 12)=23.1, P<0.0004] doses of CIS (FIG. 1). Indeed, Fisher's LSD post hoc test showed that the 10 mg/kg-treated shrews ate significantly more worms during day 1 relative to both night 1 (P<0.04) and day 2 (P<0.01), (FIG. 1). A similar pattern of effect was seen for the 20 mg/kg group (P<0.03 and P<0.0001, respectively) for these observation periods.

There was no such time effect, however for the 0 mg/kg and 5 mg/kg treatment groups. There were also significant differences across CIS doses during day 1 [F (3, 27)=6.82, P<0.0014], night 1 [F (3, 27)=11.23, P<0.0001] and day 2 [F (3, 27)=14.06, P<0.0001]. Indeed, Fishers LSD post hoc analysis revealed that the 10 mg/kg CIS-treated animals ate significantly more (P<0.02), while the 20 mg/kg group ate less (P<0.048) than the control (0 mg/kg) group during day 1 (FIG. 1). During the first night, the 5 mg/kg group ate significantly more (P<0.03), while the 20 mg/kg-treated shrews ate significantly less (P<0.001) than controls. During day 2, both the 10 mg/kg (P<0.03) and 20 mg/kg (P<0.0001) groups ate significantly less than the vehicle-treated controls.

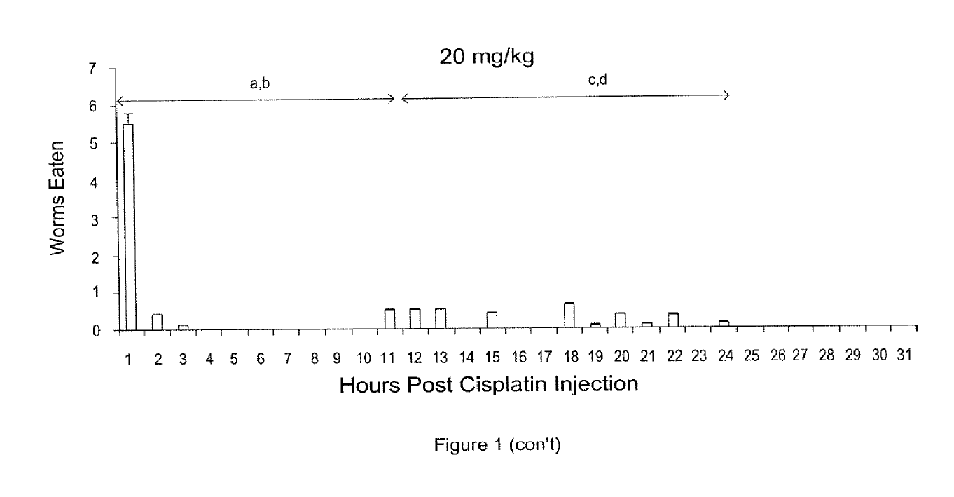

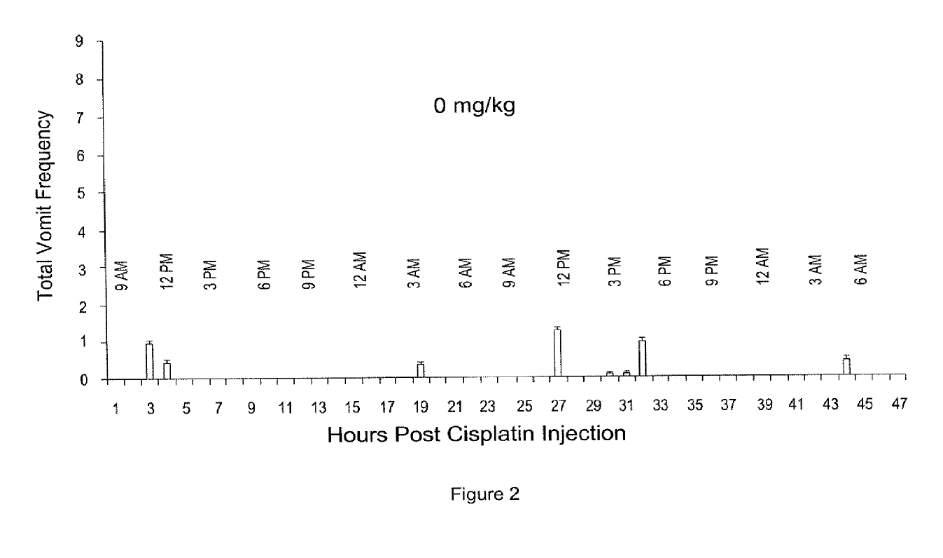

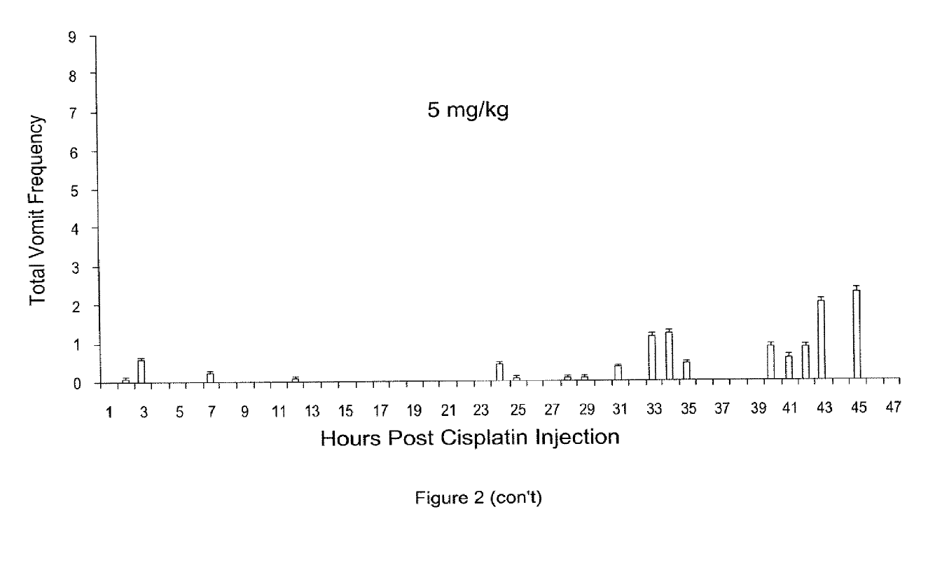

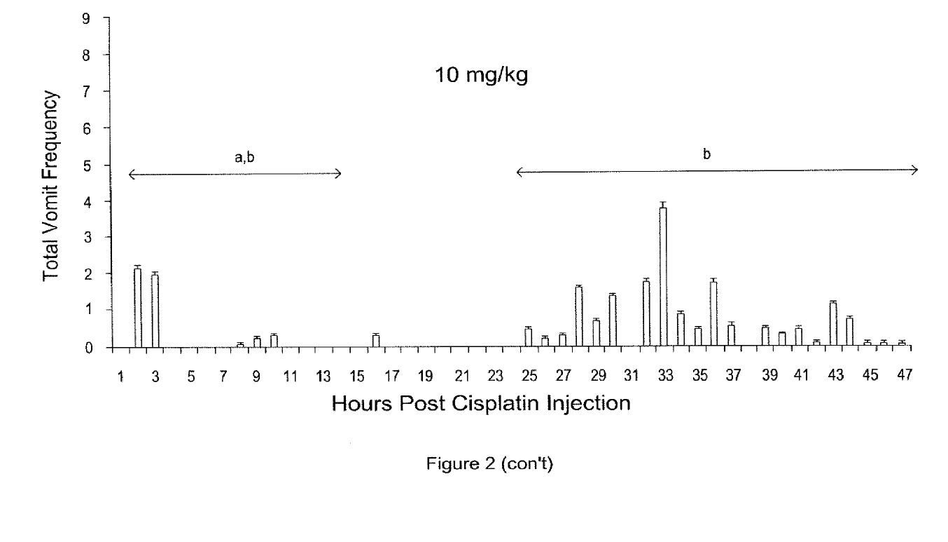

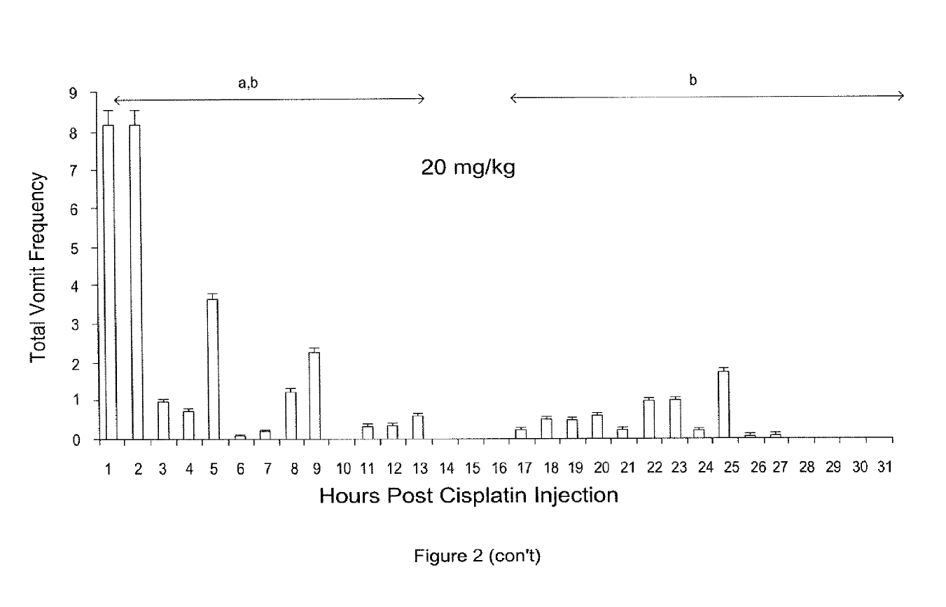

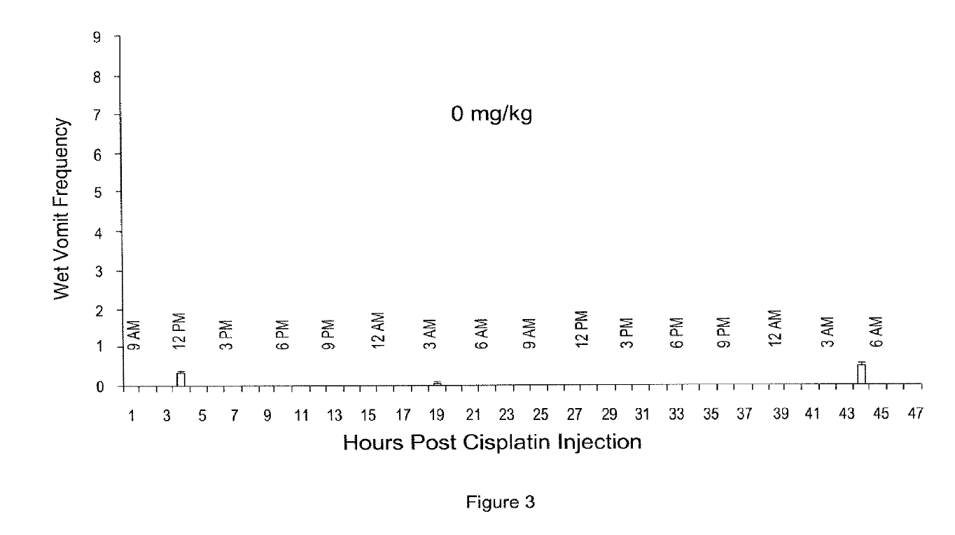

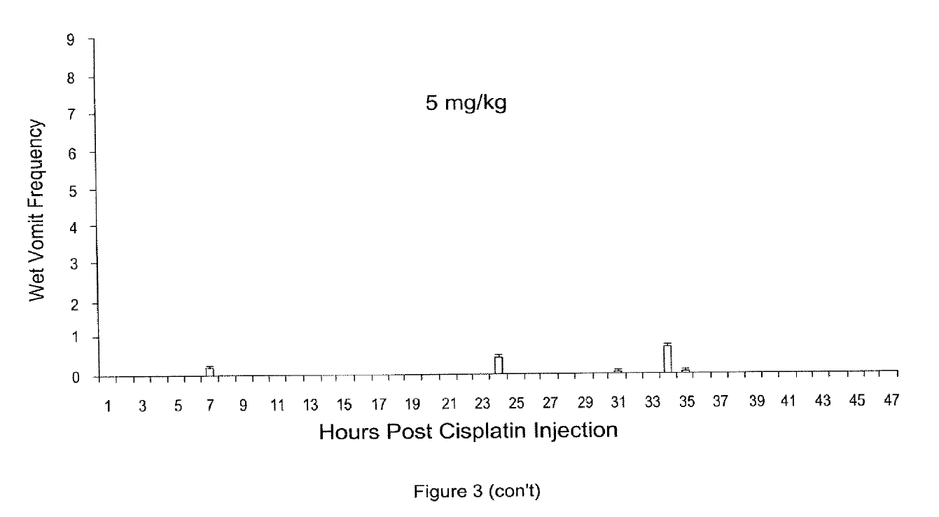

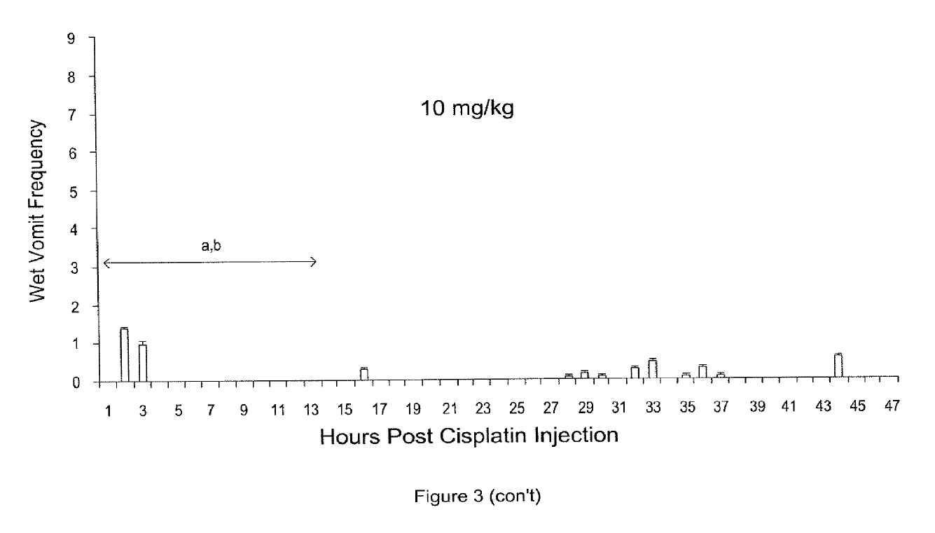

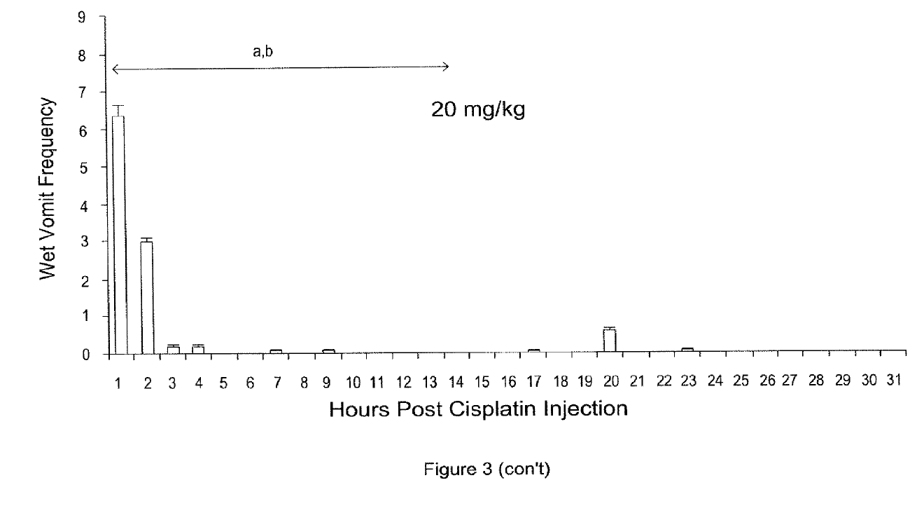

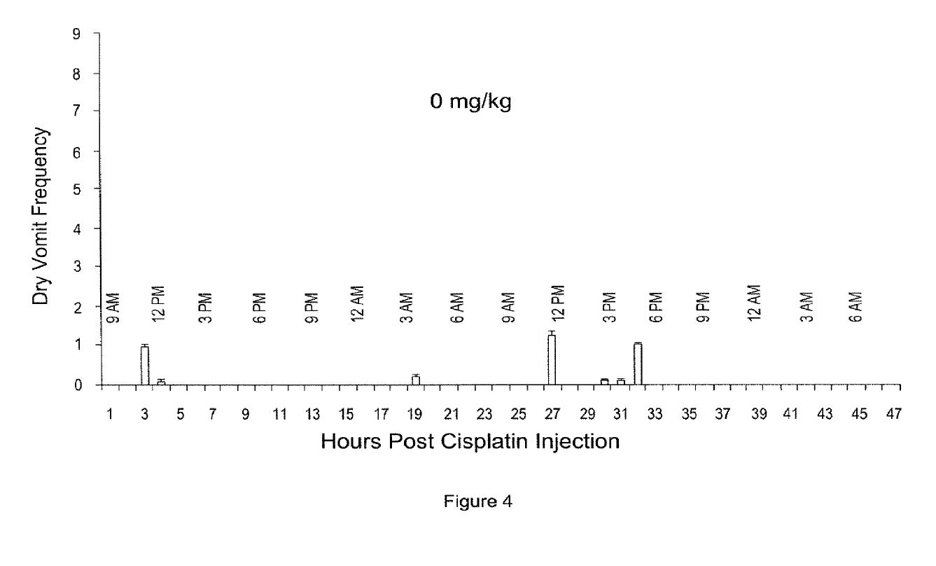

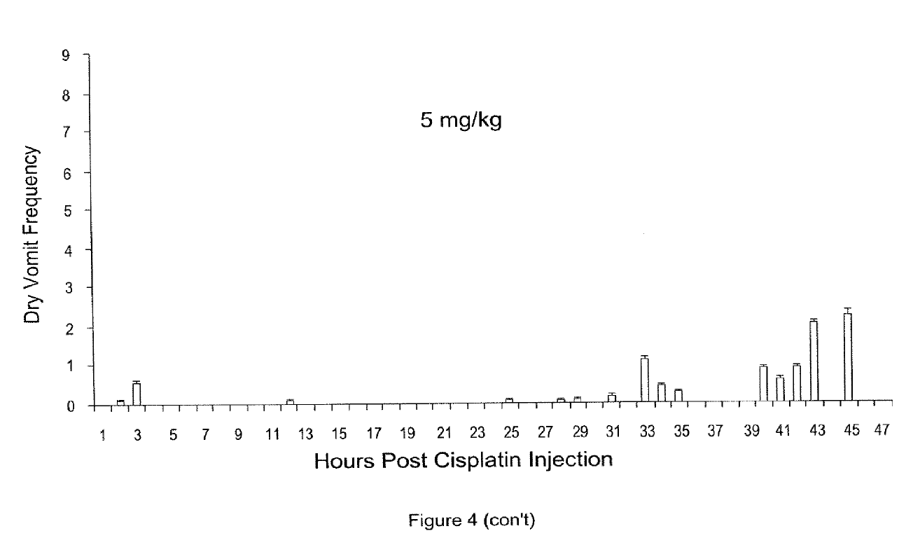

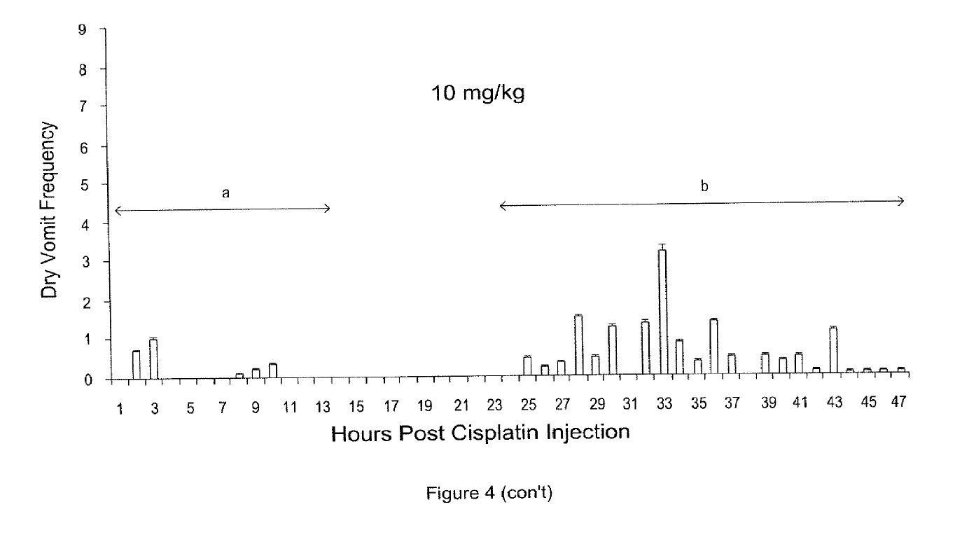

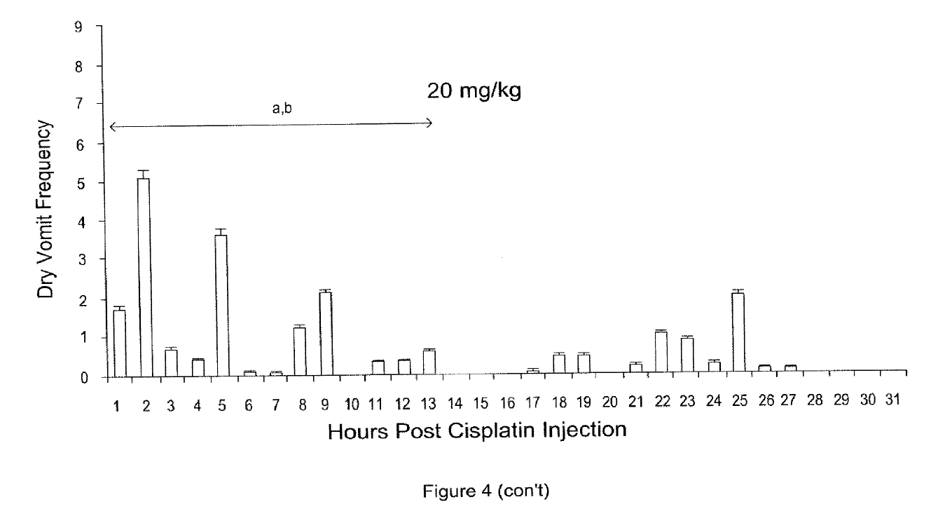

The vomiting episodes are presented in terms of total vomits (FIG. 2), wet vomits (FIG. 3) and dry vomits (FIG. 4). Some shrews in the vehicle-injected control group tended to vomit occasionally which were likely due to rapid eating and often times were of dry nature (mainly mouth openings) and this occurred in a random manner throughout the 47 hour-observation period (FIGS. 2 to 4). The frequency of vomiting per hour increased following CIS treatment. The 5 mg/kg dose exhibited a pattern of total vomiting (FIG. 2) with smaller mean frequencies (0.25 to 0.5 episodes per hour) within the first 13 hour of treatment, then a quiescent period of no emesis up to 23 hours post treatment (i.e., 14 hours to 24 hours observation), followed by more robust total mean vomiting frequencies (0.5 to 2.5 episodes per hour) throughout the remainder of the observation up to 47 hours post-treatment, which represents the delayed phase (i.e., from 24 to 47 hours post-CIS injection). The 5 mg/kg dose caused few episodes of wet vomits during the second phase and only one episode during the seventh hour of the first phase (FIG. 3). On the other hand, dry vomiting contributed for the majority of total vomits caused by the 5 mg/kg dose of CIS in both phases of emesis (FIG. 4). The 10 mg/kg dose of CIS caused more defined total episodes of vomiting (mean±SEM per hour) both during the immediate and delayed phases in the time frame described above (FIG. 2). Indeed, maximal vomit frequencies occurred between the second and third hours of CIS exposure for the immediate phase and varied from 28 to 37 hours of exposure during the delayed phase in a well defined cluster, being maximal at 33 hour post-CIS treatment.

Wet vomits also maximally contributed between second and third hours of exposure in the immediate phase and at 33 hour during the delayed phase. The 20 mg/kg CIS dose induced both phases of total emesis more rapidly (FIG. 2) and wet vomits accounted for the majority of observed emesis during the first 2 hours of exposure (FIG. 3). Dry vomits accounted for other episodes of vomiting during the remainder of first phase and nearly all of the delayed phase (FIG. 4). This CIS dose was toxic and animals did not survive the 47 hour observation period and thus observation was terminated during the second night of treatment. Therefore, the emesis data is only presented up to 31 hours of observation for the 20 mg/kg CIS dose.

The two-way repeated measures of analysis of variance of total frequency of vomiting indicated a significant overall effect between the observation periods (immediate and delayed) and CIS doses [F (3, 28)=13.3, P<0.001]. In addition, significant main effect differences in the mean frequency of total vomiting occurred between the two phases for the 10 mg/kg (delayed N immediate) [F (1, 7)=8.2, P<0.038] and 20 mg/kg doses (immediate N delayed) [F (1,7)=13.02, P<0.009], while the phase difference for the 5 mg/kg dose was outside the significance range (P=0.074) (FIG. 2). There were also significant CIS dose effects in the total frequency of emesis in both the immediate [F (3, 28)=12.45, P<0.0001] and delayed phases [F (3, 28)=8.1, P<0.029]. Post hoc analysis indicated that the 10 mg/kg (P<0.03 and P<0.045) and 20 mg/kg doses of CIS (P<0.0001 and P<0.045) were significantly different from their corresponding vehicle controls in both the immediate and delayed phases, respectively (FIG. 2) Likewise, analysis of wet vomiting indicated a significant overall effect between observation periods and CIS doses [F (3, 28)=33.1, P<0.0001] as well as main dose effects for the 10 mg/kg (immediate N delayed) [F (1, 7)=5.8, P<0.025] and 20 mg/kg (immediate N delayed) [F (1, 7)=53.5, P<0.0002] CIS doses between the two phases of emesis (FIG. 3). In the immediate phase there was a significant dose difference [F (3, 28)=30.07, P<0.0001] and post hoc analysis revealed significantly greater frequencies of wet emesis for the 10 mg/kg (P<0.045) and 20 mg/kg (P<0.0001) doses of CIS relative to the control group (FIG. 3). In the delayed phase there was no significant dose effect for wet vomiting. Two-way repeated measures of analysis of variance of dry vomiting also indicated a significant overall effect between vomit phases and CIS doses [F (3, 28=5.45, P<0.0044], as well as main differences in the immediate and delayed phases for the 10 mg/kg (immediate<delayed) [F (1, 7)=5.09, P<0.05] and 20 mg/kg CIS doses (immediate N delayed) [F (1, 7)=5.59, P<0.045] (FIG. 4). In the immediate phase, there was a significant difference among CIS doses [F (3, 28)=5.32, P<0.005] and post hoc analysis indicated the 20 mg/kg (P<0.0025) CIS dose caused significantly greater frequencies of dry vomiting relative to the vehicle treated control group (FIG. 4). The delayed phase also exhibited significant differences in the frequency of dry vomiting among CIS doses [F (3, 28)=5.9, P<0.04] with the 10 mg/kg CIS causing greater frequencies of dry emesis (P<0.04) relative to the vehicle-treated control group (FIG. 4).

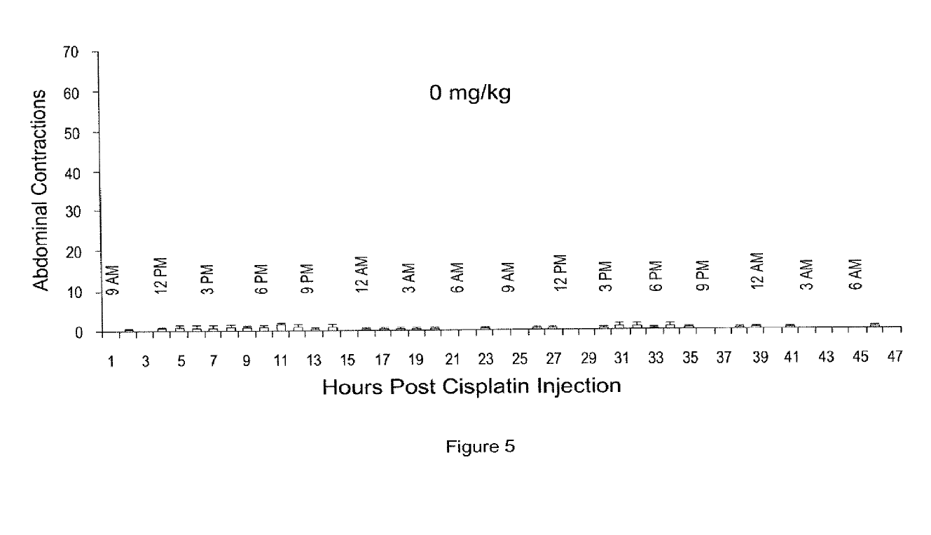

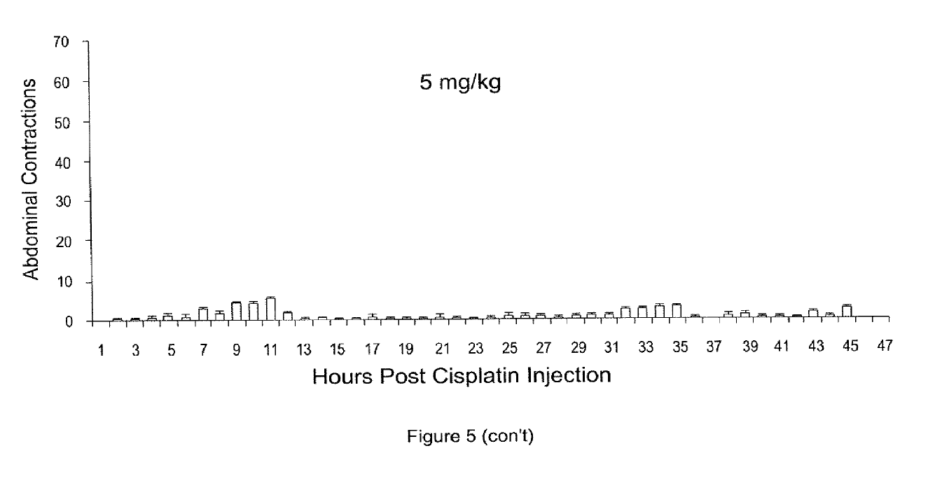

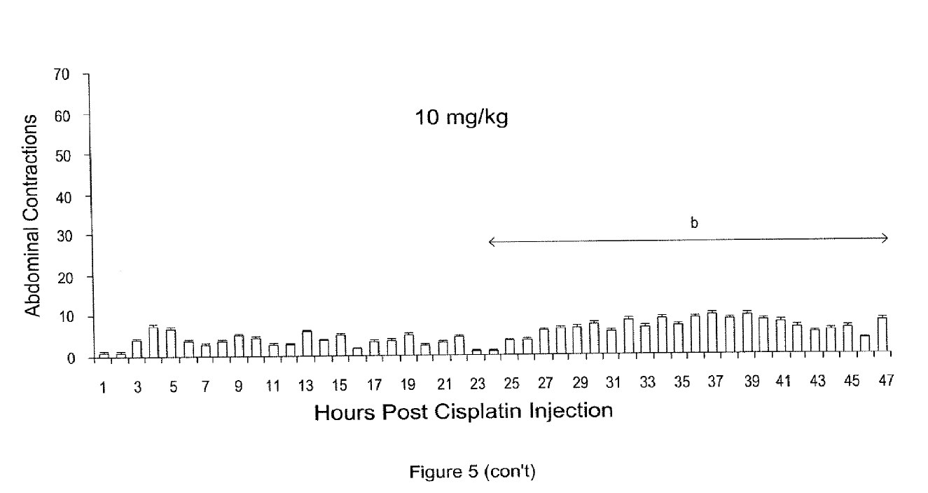

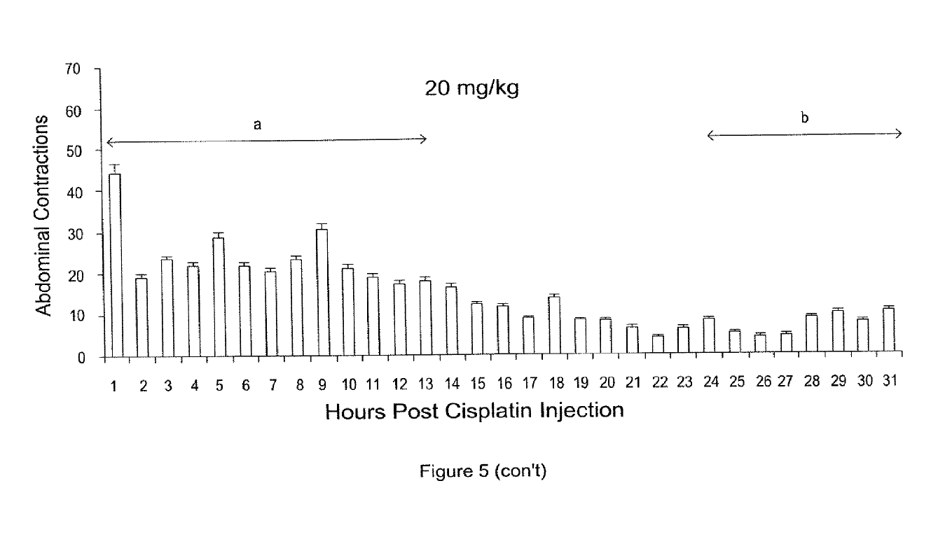

Vehicle-treated shrews exhibited very low frequencies of abdominal contractions throughout the 47 hour observation period (FIG. 5). CIS increased the frequency of contractions in a dose-dependent manner by 2-fold to 40-fold throughout the observation period in a dose-dependent manner. There was a clear maximal frequency, however, between the third and fifth hour of CIS (10 mg/kg) exposure for the immediate phase, and a second maximum between 31 hour to 47 hour of exposure, corresponding to the delayed phase of emesis (FIG. 5). The frequency of contractions was statistically analyzed in the same manner of emesis data corresponding to the time parameters set for the immediate and delayed phases of emesis.

The two-way repeated measures of analysis of variance of frequency of abdominal contractions indicated a significant overall effect between the observation periods (immediate and delayed phases) and CIS doses (0, 5, 10, and 20 mg/kg doses) [F (3, 28)=9.14, P<0.0002] (FIG. 5). Also, there were significant main differences in the mean frequency of contractions between the immediate and delayed vomiting phases in the 20 mg/kg dose [F (1, 7)=9.34, P<0.02]. Both in the immediate [F (3, 28)=11.5, P<0.000] and delayed phases [F (3,28)=5.83, P<0.003], there were significant CIS dose effects. Indeed, post hoc analysis indicated that relative to the vehicle-treated control group, the 20 mg/kg CIS dose caused a significantly greater (P<0.0001) mean frequency of contractions during the immediate phase, while both the 10 mg/kg (P<0.003) and 20 mg/kg (P<0.003) groups exhibited greater frequencies of contractions during the delayed phase (FIG. 5).

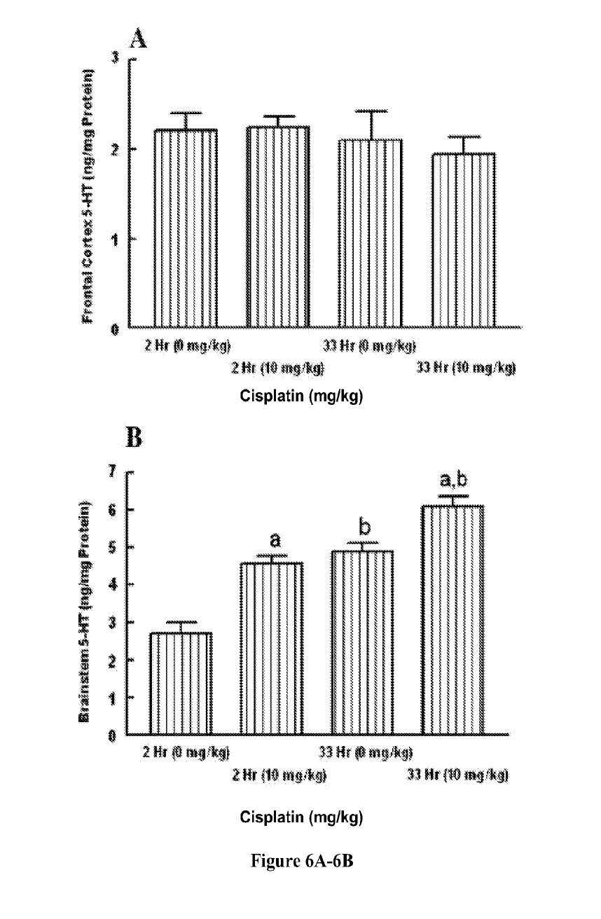

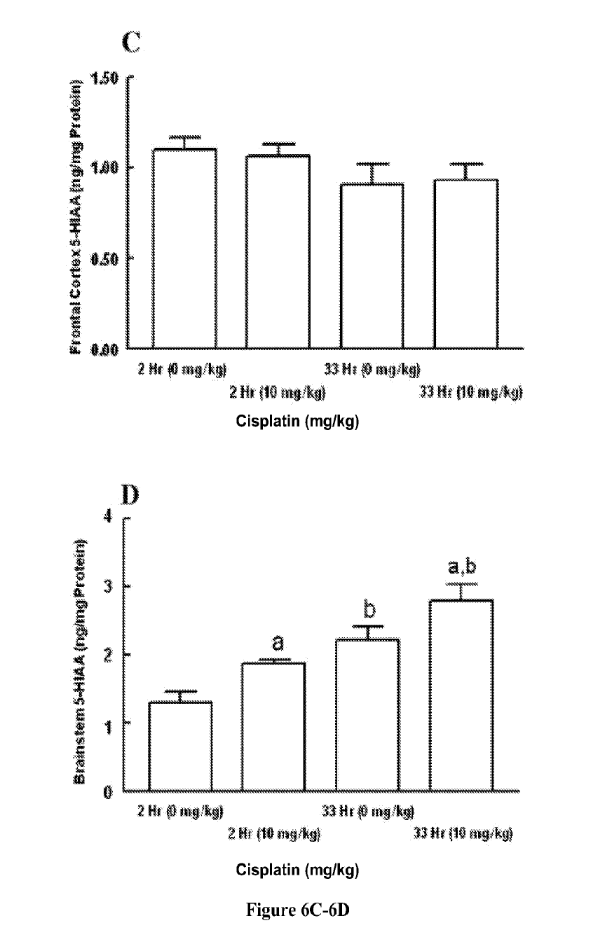

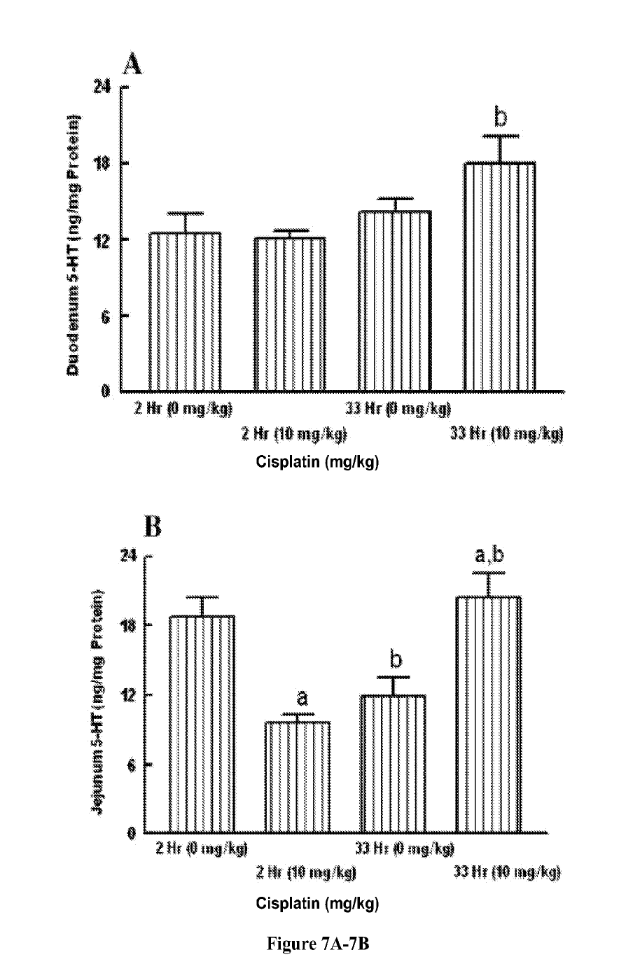

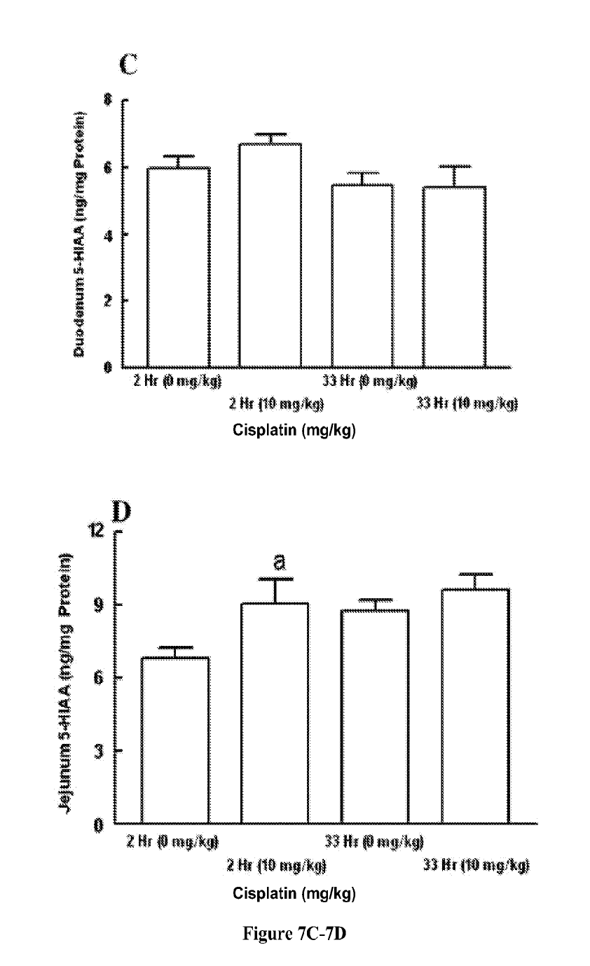

Two-factor analysis of serotonin levels in shrew frontal cortex for CIS dose (0 mg/kg and 10 mg/kg) and exposure time (2 hour and 33 hour) resulted in no significant overall effect (FIG. 6A). A similar analysis of serotonin levels in the shrew brainstem exhibited a significant overall effect, however [F (3, 31)=28.5, P<0.0001] (FIG. 6B). Analysis of main effects revealed significant dose [F, (1, 31)=35.2, P<0.0001] and time effects [F (1, 31)=52.01, P<0.0001, but no significant interaction between dose and time. Post hoc test showed that relative to their corresponding vehicle-treated controls, CIS caused significant increases in brainstem 5-HT tissue levels both at 2 hour (68.2%, P<0.0001) and 33 hour (25%, P<0.002) (FIG. 6B). In addition, brainstem 5-HT levels for both 0 mg/kg and 10 mg/kg CIS doses during the 33 hour exposure were significantly greater (69.6% increase, P<0.0001 and 33.9% increase, P<0.0002, respectively) than their corresponding values at 2 hour. Analysis of duodenal 5-HT tissue levels revealed a significant overall effect [F (3, 30)=3.62, P<0.024] (FIG. 7A).

The duodenal 5-HT concentrations were not significantly altered by CIS, however, because there was no dose effect, but there was a significant time effect [F (1, 30)=7.74, P<0.009]. Indeed, the mean 5-HT concentration at 33 hours in CIS-treated animals was significantly greater (48.5% increase) than its corresponding value at the 2 hour CIS exposure (P<0.009). Two-way analysis of jejunal 5-HT concentration exhibited a significant overall difference [F (3, 32)=11.3, P<0.0001] (FIG. 7B). Analysis of main effects indicated a significant overall interaction between CIS doses and exposure periods [F (1, 32)=31.98, P<0.0001], but exhibited no dose or time effect. Because of this interaction, the data were re-evaluated via one way analysis of variance. Thus, relative to their corresponding controls, CIS significantly (P<0.002) reduced jejunal tissue concentration by 49.1% at 2 hour but increased it by 41.8% (P<0.0007) at the 33 hour exposure (FIG. 7B). In addition, for the 0 mg/kg dose, the 5-HT concentration at 2 hour was greater (36.7%, P<0.004) than at 33 hours, although the reverse was true for the 10 mg//kg CIS dose (114.5% smaller, P<0.0001).

Two-way analysis of variance indicated that CIS administration failed to significantly affect the basal levels of 5-HIAA in the frontal cortex either at 2 hour (1.1±0.07 vs. 1.06±0.7) or 33 hour exposure (0.91±0.11 vs. 0.93±0.09) relative to their corresponding vehicle-treated control groups (FIG. 6C). Analysis of 5-HIAA levels in the shrew brainstem revealed significant overall effects [F (3, 31)=11.88, P<0.0001] (FIG. 6D). Analysis of main effects showed significant dose [F (1, 31)=9.91, P<0.004] and time effect [F (1, 31)=26.64, P<0.0001], but no significant dose and time interaction. Post hoc test showed that relative to their corresponding vehicle-treated controls, CIS significantly increased brainstem tissue 5-HIAA levels both at 2 hour (43.8%; P<0.03) and 33 hour (26.7%, P<0.032). In addition, 5-HIAA levels were greater (71.5%, P<0.001 and 49.2%, P<0.008, respectively) at 33 hour relative to their corresponding values at 2 hour for both vehicle- and CIS-treated groups. The duodenal tissue concentration of 5-HIAA was not significantly altered by CIS (FIG. 7C). Two-way analysis of jejunal 5-HIAA concentration indicated a significant overall effect, however [F (3, 33)=3.22, P<0.035] (FIG. 7D). Analysis of main effects indicated a significant CIS dose effect [F (1, 31)=5, P<0.032] but no time- or interaction effect. Indeed, post hoc analysis showed that CIS significantly increased (32.4%, P<0.023) jejunal 5-HIAA concentration at 2 hour relative to its corresponding control (FIG. 7D).

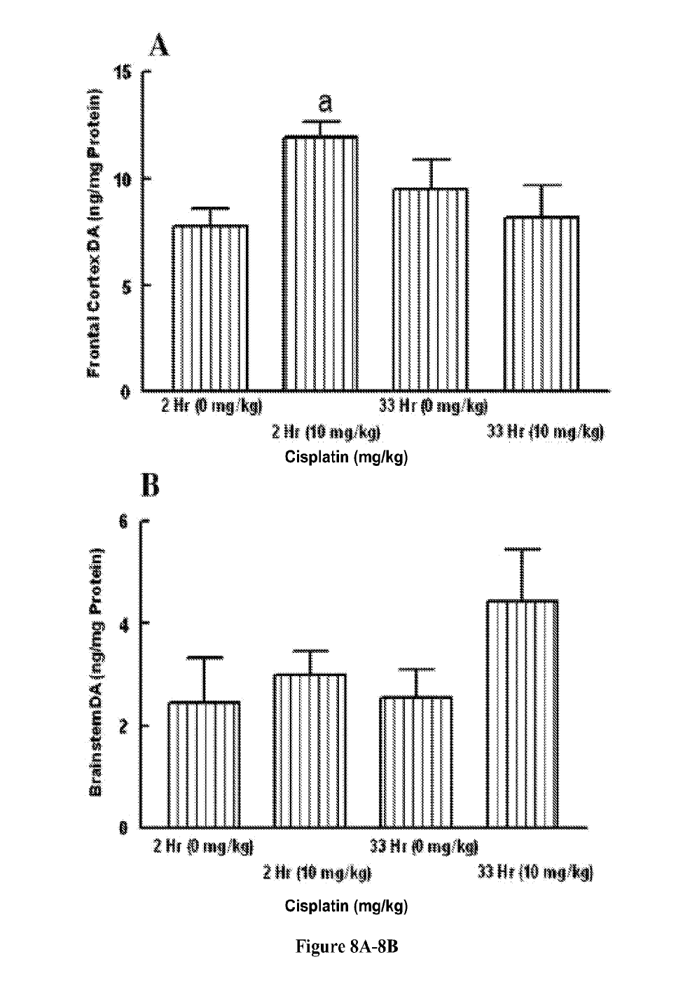

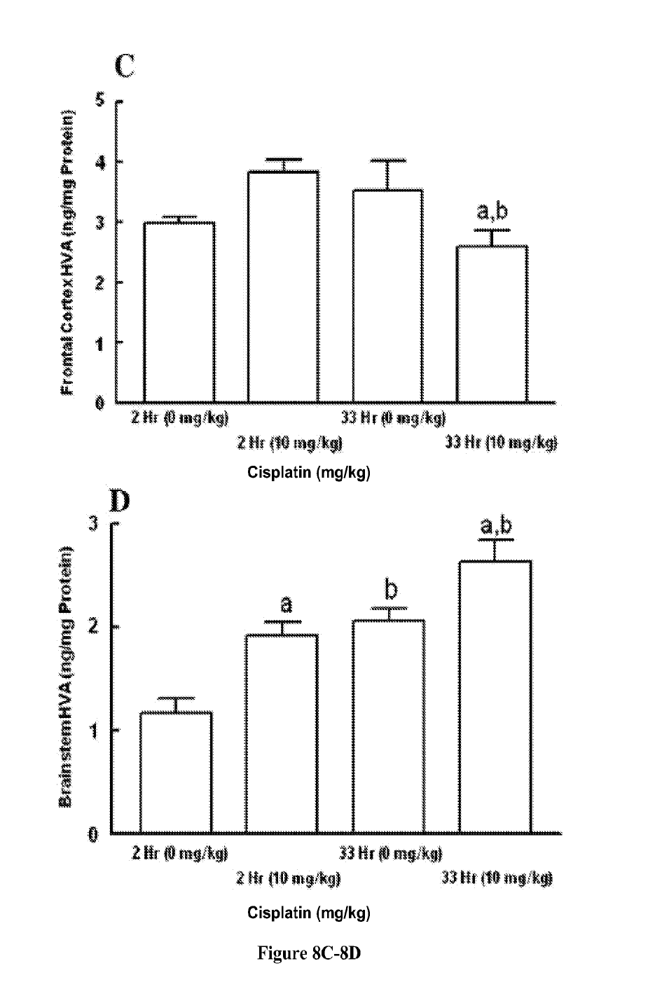

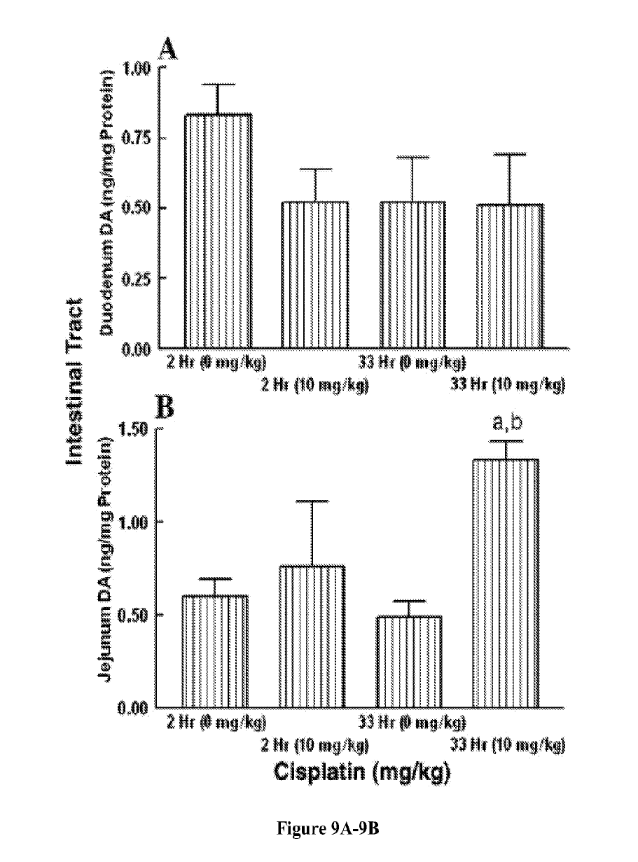

Two-factor analysis of dopamine concentrations in shrew frontal cortex indicated a significant overall interaction [F (3, 31)=5.14, P<0.03] but no dose or time effect. Thus, a subsequent one-way analysis of the data indicated a significant increase in dopamine tissue level (P<0.025) in the 10 mg/kg CIS group during the 2 hour exposure relative to its control group (FIG. 8A). Such analysis of dopamine levels in either the brainstem (FIG. 8B) or duodenum (FIG. 9A) for CIS dose (0 and 10 mg/kg) and time (2 and 33 hour) resulted in no overall significant effect. On the other hand, such analysis indicated a significant overall effect for jejunal dopamine levels [F (3, 31)=3.35, P<3.35, P<0.03] and analysis of main effects showed a significant dose effect [F (1, 31)=6.30, P<0.017] but no time- or interaction effect. Indeed, CIS increased jejunal dopamine concentration by 171% (P<0.007) and 121% (P<0.06) at 33 hour relative to both its corresponding 0 mg/kg control value at 33 hour and the 0 mg/kg control group at 2 hour, respectively (FIG. 9B).

Two-way analysis of HVA concentration in shrew frontal cortex for CIS dose (0 and 10 mg/kg) and time (2 and 33 hour) indicated a significant overall effect [F (3, 31)=3.03, P<0.045] (FIG. 8C). Analysis of main effects showed significant interaction [F (1, 31)=47.42, P<0.011] but no dose or time effect. A one-way analysis of each factor indicated that CIS caused a significant decrease in HVA concentration relative to its corresponding vehicle control in frontal cortex (35.9%, P<0.04) at 33 hour exposure (FIG. 8C), while HVA concentration tended to increase relative to corresponding vehicle-treated control at 2 hours, but the change did not achieve significance (FIG. 8C). In addition, HVA tissue level was significantly less (32.4%, P<0.009) at 33 hour than 2 hour exposure for the 10 mg/kg CIS dose (FIG. 8C). A similar two-way analysis of HVA in the brainstem revealed a significant overall effect [F (3, 31)=13.48, P<0.0001] (FIG. 8D). Analysis of main effects revealed a significant dose [F (1, 31)=16.81; P<0.0003] and time [F (1, 31)=24.71, P<0.0001] effect but no interaction. Post hoc analysis indicated that CIS significantly increased HVA levels in the brainstem both at 2 hour (65.5%, P<0.003) and 33 hours (27.7%, P<0.02) relative to their corresponding control levels (FIG. 8D). In addition, brainstem HVA levels were greater in both 0 mg/kg (77.69%, P<0.000) and 10 mg/kg (37% P<0.04) CIS-treated groups at 33 hour relative to their corresponding dose levels at 2 hour. HVA concentration in the intestine could not be evaluated due to an unknown interfering peak.

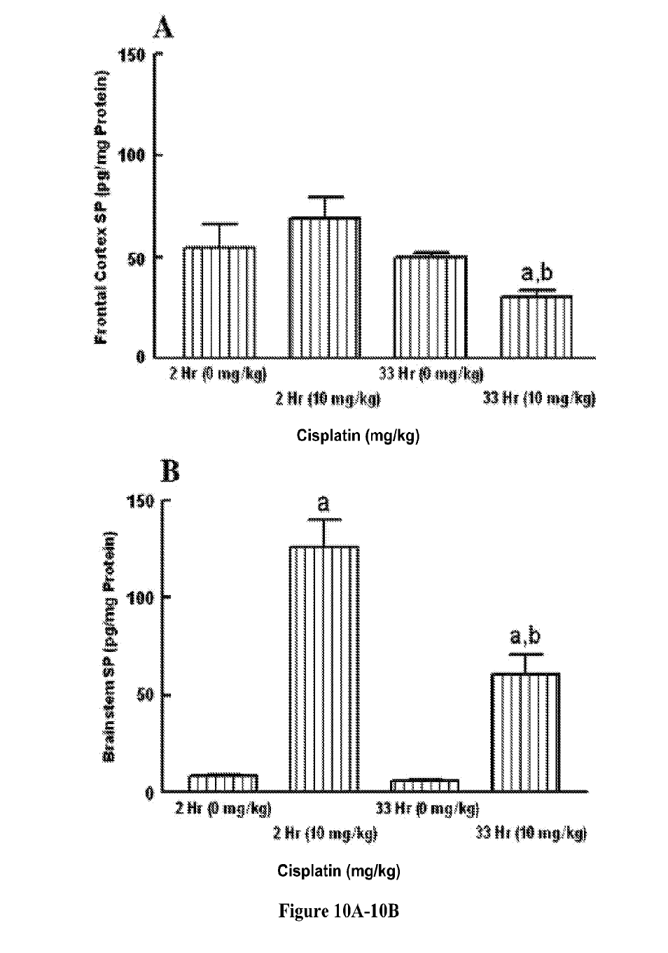

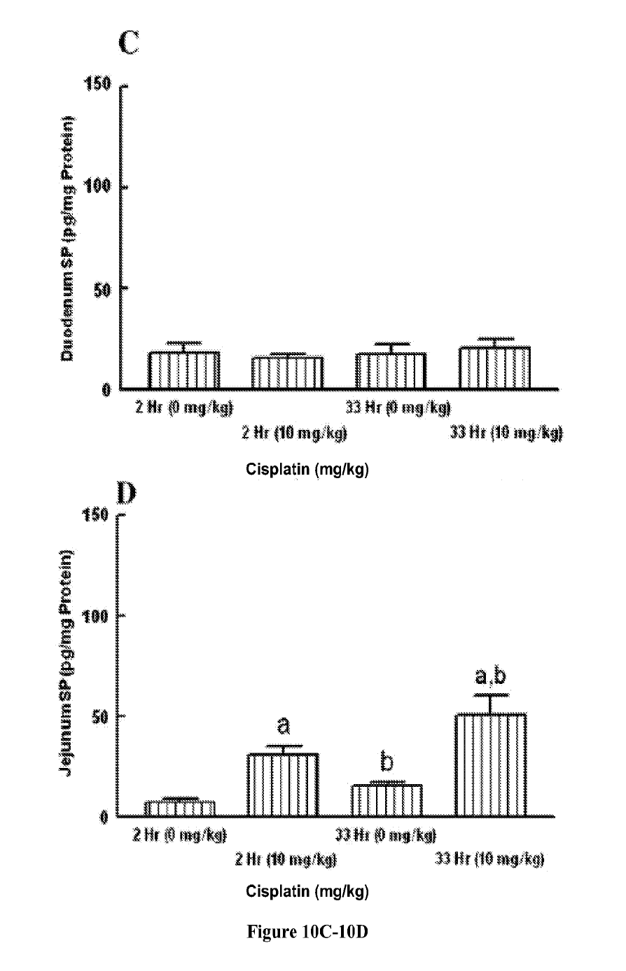

Two factor analysis of variance for SP tissue levels in shrew frontal cortex indicated a significant overall effect [F (3, 26)=4.4, P<0.012] (FIG. 10A). Analysis of main effects showed significant time [F (1, 26)=8.3, P<0.008] and interaction effects but no significant dose effect. Because of this interaction, a one-way analysis of variance was utilized to re-evaluate the data. Post-hoc analysis revealed no significant difference in frontal cortex SP tissue levels between vehicle and CIS groups at 2 hour, but CIS significantly reduced (39.2% decrease, P<0.001) SP levels at 33 hour relative to its corresponding vehicle-treated controls. In addition, SP tissue levels were significantly lower in CIS-group at 33 hour vs. 2 hour (FIG. 10A). Two-way analysis of brainstem SP concentration also exhibited an overall significant effect [F (3, 26)=47.1, P<0.0001] (FIG. 10B). Analysis of main effects revealed significant dose [F (1, 26)=106.1, P<0.0001], time [F (1, 26)=16.6, P<0.0001] and interaction effects [F(1,26=4.9, P<0.001]. Indeed, Post hoc analysis revealed significantly higher (1396% and 956% increases, P<0.0001 and P<0.0002, respectively) SP levels in CIS-treated groups during the 2 hour and 33 hour relative to their corresponding vehicle-treated control groups (FIG. 10B). Furthermore, brainstem mean SP level was significantly lower in the 33 hour CIS group (100%, P<0.002) compared to the 2 hour CIS group (FIG. 10B). Two-way analysis of variance for duodenal SP tissue levels failed to exhibit a significant effect (FIG. 10C), but jejunal SP concentrations revealed an overall significant effect [F (3, 26)=13.11, P<0.0001] (FIG. 10D). Furthermore, analysis of main effects of jejunal SP concentrations revealed significant dose [F (1, 27)=29.97, P<0.0001] and time effects [F (1, 27)=6.96, P<0.01] but no interaction. Post hoc analysis revealed that CIS caused significant increases in SP levels [333% (P<0.0001) and 226.5% (P<0.0001), respectively] at both 2 and 33 hour observation periods relative to their corresponding vehicle-treated controls. In addition, the SP level in vehicle-treated control group at 33 hour was significantly higher than its corresponding value at the 2 hour treatment period (118% increase, P<0.0001).

The present invention thus provides for an animal model that is particularly useful in studying emesis. For example, temporal developments of CIS's immediate and delayed emetic phases were characterized in the Least Shrew, which were dose- and time-dependent events. CIS (10 mg/kg, i.p.) produced both emetic phases with corresponding maximal mean frequencies occurring respectively at 2-3 hour and 33 hour post exposure. The Least Shrew appears to be a rapid and robust emesis model for screening the efficacy of antiemetics against both phases of CIV in less than 47 hour. The present neurotransmitter studies suggest that the two peak phases of CIV are simultaneously associated with increases in 5-HT, dopamine, and SP turnover in both the brainstem and in specific portions of the small intestine such as the jejunum. Thus, both central and peripheral mechanisms appear to contribute to the emetic actions of each of the discussed neurotransmitters, albeit there is overlap as well as variation in the extent of contribution of each of these anatomic and neurochemical emetic parameters in the mediation of each phase of CIV. Moreover, although the presence of such neurotransmitters is a necessary prerequisite for emetic activity, their release in the cited loci per se does not guarantee their emetic activity either at a specific site or at a given CIV phase.

In the absence of a suitable animal model, the discovery of emetic action of SP and the role of brain stem NK₁ receptors in vomiting is mainly based upon indirect evidence. The present invention provides a model in which the direct emetic effects of systemic SP and several selective brain penetrating and non-penetrating NK₁ receptor agonists, as well as the involvement of tachykinin NK₁ receptors, NK₂ receptors, and NK₃ receptors are systematically characterized in a vomiting species, the Least Shrew NK₁ receptor model of emesis.

More specifically, aspects of the present Least Shrew emetic model (a) demonstrate whether intraperitoneal (i.p.) administration of either SP, brain penetrating or non-penetrating NK₁, NK₂, or NK₃ receptor agonists induce vomiting and/or scratching in the Least Shrew (C. parva) in a dose-dependent manner; (b) show whether these effects are sensitive to the above selective NK₁, NK₂ and/or NK₃ receptor antagonists; (c) show whether an exogenous emetic dose of SP (50 mg/kg, i.p.) can penetrate into the shrew brain stem and frontal cortex; (d) demonstrate whether GR73632 (2.5 mg/kg, i.p.)-induced activation of NK₁ receptors would increase Fos-measured neuronal activity in the neurons of both the brain stem emetic nuclei and the enteric nervous system of the gut; and (d) show whether selective ablation of peripheral NK₁ receptors can affect emesis produced by GR73632.

The embodiments provide an animal model in which SP resulted only in emesis, and GR73632 caused both emesis and scratching behavior in a dose-dependent fashion, and these effects were sensitive to NK₁ receptor antagonists, but not to NK₂ or NK₃ receptor antagonists. Neither the non-penetrating NK₁ receptor agonists, nor NK₂ and NK₃ receptor-selective agonists caused a significant dose-dependent behavioral effect. An emetic dose of SP selectively and rapidly penetrated the brain stem but not the frontal cortex. Systemic GR73632 increased Fos activity in the enteric nervous system of the gut, as well as in the medial subnucleus of nucleus tractus solitarius and the dorsal motor nucleus of the vagus, but not in the area postrema. Peripheral ablation of NK₁ receptors attenuated the ability of GR73632 to induce a maximal frequency of emesis and shifted its percent animal vomiting dose-response curve to the right. The NK₁ receptor-ablated shrews were able to exhibit scratching behavior in response to systemic GR73632-injection. These results, for the first time, affirm both a cardinal role for central NK₁ receptors in the ability of SP to induce vomiting, and a facilitating role for the gastrointestinal NK₁ receptors to rapidly expel vomit. Importantly, the Least Shrew is a specific and rapid behavioral animal model to screen concomitantly both the CNS penetration and the antiemetic potential of tachykinin NK₁ receptor antagonists.

Substance P (SP), neurokinin A, and neurokinin B are endogenous members of the tachykinin family of neuropeptides, which preferentially interact with three related G-protein-coupled receptors, the neurokinin NK₁, NK₂, and NK₃ receptors, respectively. Severini et al., 54 Pharmacol. Rev. 285-322 (2002). SP has been implicated as a primary afferent neurotransmitter via NK₁ receptors in various noxious stimuli including emesis. Andrews & Sanger, 2 Curr. Op. Pharmacol. 650-56 (2002). Indeed, SP-like immunoreactivity, high concentrations of the peptide, and significant densities of NK₁ receptors are found in several anatomical substrates of emesis: (a) the DVC (Amin et al., 126 J. Physiol. 596-618 (1954); Andrews & Rudd, 164 HANDBOOK EXP. PHARMACOL. 359-440 (Holzer, ed. Springer-Verlag, Berlin, 2004)); (b) the peripheral enteric nervous system of the GIT, which is involved in the genesis of abnormal motility patterns in emesis (Andrews & Rudd, 2004): and (c) the enterochromaffin cells (EC) of intestinal mucosa (Sjolund et al., 85 Gastroenterology 1120-30 (1983)). The latter is of particular importance because the release of both serotonin and SP from EC cells could play important roles in the induction of immediate and delayed phases of emesis produced by cytotoxic anti-cancer therapies. Veyrat-Follet et al., 1997.

Moreover, intravenous (i.v.) administration (Carpenter et al., 43 Federation Proc. 2952-54 (1984)), or central injection of SP in the NTS (Gardner et al., 112 Br. J. Pharmacol. 516P (1994)), induce emesis respectively in both conscious dogs and ferrets, while its topical application to the AP elicits retchings in anesthetized ferrets (Andrews & Rudd, 2004). In general, it is largely believed that the site of anti-emetic action of NK₁ receptor antagonists is in the CNS because: (a) brain penetration is required for their anti-vomiting activity (Rupniak et al., 326 Eur. J. Pharmacol. 201-09 (1997)); (b) intracisternal administration or direct injection of CNS penetrant or nonpenetrant NK₁ receptor antagonists into the vicinity of the NTS prevents emesis produced by the peripheral administration of CIS (Tattersal et al., 35 Neuropharmacol. 1121-29 (1996); Gardner et al., 1994; Tanihata et al., 461 Eur. J. Pharmacol. 197-206 (2003)); (c) NK₁ receptor antagonists possess broad spectrum antiemetic activity against both central and peripheral emetogens (Megens et al., 302 J. Pharm. Exp. Therap. 696-709 (2002); Singh et al., 321 Eur. J. Pharmacol. 209-26 (1997); Rudd et al., 366 Eur. J. Pharmacol. 243-52 (1999); Lucot et al., 120 Br. J. Pharmacol. 116-20 (1997)); and (d) there is strong correspondence in the rank order of potency between NK₁ receptor antagonists ID₅₀ₛ for their antiemetic activity against CIS-induced or loperamide-induced emesis in ferrets, and their ability to suppress centrally-mediated foot tappings in gerbils induced by central injection of NK₁ receptor selective agonists (Megens et al., 2002; Rupniak et al., 1997; Singh et al., 1997). The latter two models have been concomitantly used as central indices for the CNS penetration, NK₁ receptor antagonist activity, and CNS-mediated antiemetic potential of NK₁ receptor antagonists.

There are significant differences between typical rodent and human NK₁ receptors, however. In general, rodent NK₁ receptors have low affinity for NK₁ receptor antagonists, and therefore, alternative animal models such as gerbils have been introduced, because such antagonists have the tissue receptor affinity profile for the NK₁ antagonists in these animals is similar to that for human NK₁ receptors. Duffy et al., 301 J. Pharmacol. Exp. Therap. 536-42 (2002); Rupniak et al., 1997.

Despite the emetic potential of SP, no one has reported on the emetic efficacy of synthetic selective NK₁ receptor agonists such as GR73632 (Hagan et al., 19 Neuropeptides 127-35 (1991)), although work has been done investigating the anti-emetic potential of NK₁ receptor antagonists, either against the early and/or delayed phase of CIS-induced emesis, or following administration of diverse centrally/peripherally-acting emetogens. Andrews & Rudd, 2004. These models are nonspecific, however, and require one to three days of constant behavioral observation, or they are not directly related to NK₁ receptor activation. In rodent models central administration of NK₁ receptor agonists induces a specific NK₁ receptor-mediated behavior analogous to foot-tapping in gerbils called scratching. Ravard et al., 651 Brain Res. 199-208 (1994).

Additionally, there are a number of inconsistencies in dogma of “central-only” site of antiemetic action of NK₁ receptor antagonists. For example, although SP appears to be an inconsistent emetogen in the ferret when administered i.v. or subcutaneously (s.c.) (Knox et al., 31 Brain Res. Bull. 477-83 (1993)), it is a potent emetogen in the dog when administered i.v. (Carpenter et al., 1984), but does not induce emesis when administered intracerebroventricularly (i.c.v.) either in the dog or cat (Wu et al., 1 Peptides 173-75 (1985); Yasnetsov et al., 103 Byulleten Eksperimental' not Biologyii I Meditsiny, 586-88 (1987)). Additionally, when administered s.c., the peripherally-acting peptide NK₁ receptor antagonist, sendide, prevents emesis in ferrets caused by i.p. injection of CIS. Minami et al., 363 Eur. J. Pharmacol. 49-55 (1998). Furthermore, the binding affinity of NK₁ receptor antagonists for either the human or ferret brain NK₁ receptors is not predictive of their rank potency order to inhibit CIS-induced retching in ferrets. Rupniak et al., 1997. Finally, intravenous administration of the NK₁ receptor antagonist, RPR 100893, was shown to be effective against CIS(i.v.)-induced emesis in ferrets but failed to reduce the NK₁ receptor (GR73632, i.c.v.)-induced foot-tapping behavior in gerbils. Rupniak et al., 1997. These disparate findings suggest a peripheral component for the vomiting activity of SP in addition to its proposed central emetic action.

Indeed, centrally- and/or peripherally-acting NK₁ receptor antagonists, such as CP99,994 or sendide, can reduce the vagal afferent discharge produced by: peripherally-administered SP or serotonin (Minami et al., 428 Eur. J. Pharmaco. 215-20 (2001); Minami et al., 1998), or by the electrical stimulation of the vagus nerve in an anesthetized ferret preparation (Watson et al., 115 Br. J. Pharmacol. 84-94 (1995)).

A major factor that has hampered further progress is the lack of a rapid and specific animal model for the study of emetogenic potential of SP and related NK₁ receptor selective agonists. One possible model to investigate the peripheral emetic component of NK₁ receptor activation is to selectively ablate peripheral NK₁ receptors via systemic administration of a saporin conjugate of the more potent and peptidase resistant analog of SP called [Sar⁹, Met (O₂)¹¹]-SP or SSP-saporin. Such immunotoxin conjugates have greater specificity, potency and duration of action and can lesion neurons expressing NK₁ receptors in the CNS. Wiley & Lappi, 277 Neuroscience Lett. 1-4 (1999); Abdala et al., 1119 Brain Res. 165-173 (2006). Despite the emetic potential of SP, no one has reported on the emetic efficacy of synthetic selective NK₁ receptor agonists such as GR73632. Hagan et al., 19 Neuropeptides 127-35 (1991).

Preliminary studies indicated that intraperitoneal injection of GR73632 can rapidly induce both emesis and the scratching behavior in the Least Shrew (C. parva). This species was introduced recently as a versatile new emesis by the present applicant. Darmani,105 J. Neurol. Transm 1143-54 (1998); Darmani et al., 106 J. Neulol. Transm. 1045-61 (1999); Darmani, 24 Neuropsychopharmacol. 198-203 (2001a)), and could potentially be utilized as a rapid and specific behavioral model for the study of both CNS penetration and the antiemetic potential of NK₁ receptor antagonists.

This present invention provides for the C. parva behavioral model, and addresses some of the discussed gaps in the current literature by (a) investigating whether peripheral administration of SP or brain penetrating and non-penetrating selective NK₁ receptor agonists can induce emesis and other behaviors (e.g., scratching) in a dose-dependent fashion in the Least Shrew; (b) pharmacologically deciphering which tachykinin receptor is responsible for the induction of the induced behaviors in the Least Shrew via the utilization of selective NK₁-, NK₂- and NK₃-receptor agonists and antagonists; (c) analyzing the increasing tissue levels of exogenous SP in the brain stem and frontal cortex following intraperitoneal injection of an emetic dose of SP; (d) examining whether Fos-measured neuronal activity increases in the emetic loci of the brain stem and gastrointestinal enteric nervous system following systemic administration of the NK₁ receptor agonist GR73632; (e) and demonstrating the possible role of peripheral NK₁ receptors in emesis following their SSP-saporin-induced ablation in the GIT.

Intraperitoneal administration of SP caused dose-dependent increases in emesis. The emetic effects of SP occur in a narrow range with maximal activity at 50 mg/kg. Lower doses failed to induce a significant increase either in vomit frequency or in the number of animals exhibiting vomiting, while a larger dose (100 mg/kg) did not further potentiate its effects. These findings confirm published data in the dog (Carpenter et al., 1984) that i.v.-administered SP is a robust emetogen at doses of 0.05 to 0.2 mg/kg. As both the source and the recipient of peptide signals, the brain could be influenced by endogenous, blood-borne peptides such as SP. Several studies have shown that low doses of exogenous SP (0.05 to 0.25 mg/kg, i.p.) induce hypothermia (Richter & Oehme, 41 Acta. Biol. Ned. Ger. 725-27 (1982)), and changes in both operant behavior (Hasenöhrl et al., 55 Physiol. Behay. 541-46 (1994)) and brain monamine levels (Boix et al., 216 Eur. J. Pharmacol. 103-07 (1992)) in rodents.

Although SP is unlikely to pass the blood-brain-barrier under physiological conditions, this peptide may, in significant amounts, gain entrance rapidly by a specific transport mechanism (Freed et al., 23 Peptides 157-67 (2001a); Chappa et al., 23 Pharma. Res. 1201-08 (2006)), and not endocytosis (Lamaziére et al., 2(2) PLos One e201 (2007)), by making use of the AP or other circumventricular organs in the CNS. The AP is located at the caudal extremity of the fourth ventricle outside the blood-brain and cerebrospinal fluid barriers in the brain stem and possesses active influx and efflux transport proteins (Begley, 48 J. Pharmacol. 136-46 (1996)), as well as sensitivity to chemicals in the blood (Ermisch et al., 5. J. Cerebral Blood Flow Meth. 350-57 (1985)). Furthermore, capillaries from the AP make vascular links with the dorsal region of the commissural subnucleus of the NTS, which itself has fenestrated capillaries with high permeability. Roth & Yamamato, 133 J. 329040 (1968); Gross et al., 259 Am. J. Physiol. R1132-38 (1990).

Although exogenously administered SP can be degraded relatively rapidly in larger species (Freed et al., 2001a; Palmieri & Ward, 755 Biochem. Biophys. Acta. 522-25 (1983)), SP seems particularly sensitive to catabolism in the Least Shrew, given that its large emetic dose (50 mg/kg, i.p.) attained maximal systemic blood serum concentration within five minutes of injection and rapidly declined to basal levels within the next five minutes Likewise, although maximal duodenal and jejunal tissue concentrations did occur within five minutes of exposure, levels significantly above baseline remained in these tissues for up to 15 minutes. SP levels increased in a time-dependent fashion in the shrew brain stem but not in the frontal cortex which indicates regionally selective entry of SP in the emetic DVC nuclei. Ultra rapid and selective entry of tritiated SP following intracarotid injection also occurs in rat circumventricular organs including the choroid plexus, but not in frontal and other cortical areas. Landgraf et al., 38 Pharmazie 108-10 (1983). As expected, the brain stem SP-tissue level required a relatively longer time to attain maximal levels (15 minutes) and then rapidly declined to basal levels over the next 15 minutes. These findings correspond well with emesis results in that the onset of first emesis occurred within one to two minutes of SP injection and the remaining episodes were scattered at various times within twenty-five minutes of exposure. Thus, the current tissue SP distribution results and behavioral findings support a rapid entry into the brain and don't conflict with published studies describing a specific carrier-mediated transport mechanism for the passage of blood SP into the brain stem. Begley, 48 J. Pharm. Pharmacol. 136-46 (1996); Chappa et al., 2006; Freed et al., 23 Peptides 157-65 (2002); Landgraf et al., 1983.

Because SP is rapidly metabolized and can simultaneously activate all three neurokinin receptors at large emetic doses, the emetic effects of more stable brain penetrating and non-penetrating NK₁ receptor-selective agonist analogs of SP, that have varied amino acid composition and peptide length, were also studied. Like SP, SarMet-SP is an undecapeptide, while ASMSP is a modified hexapeptide, and GR73632 is a modified pentapeptide. The first two agonists, at 10 to 20 mg/kg doses, caused emesis in only 30%-50% of tested shrews, and their emetic effects neither achieved significance nor dose-dependence. Thus, the inability of the latter compounds to induce such behaviors appears to be due to either poor penetration into the brain stem, or species differences in the primary sequence of NK₁ receptors. Chappa et al., 23 Pharmaceut. 1201-08 (2006). Poor CNS penetration appears to be a more important factor since such agonists are normally administered centrally to induce motor behaviors (Engberg et al., 73 Biochem. Pharmacol. 259-69 (2007); Yip & Chahl, 94 Neuorsci. 663-73 (1999)), and species differences seem not to affect the potency/efficacy of NK₁ receptor agonists despite being able to drastically alter the affinity of small molecule NK₁ receptor antagonists (Engberg et al., 2007). The finding, herein, that the brain penetrating NK₁ receptor agonist GR73632 (Hagan et al., 1991), caused dose-dependent emesis, with significant vomiting occurring at 1/20 to 1/40 of emetic doses of SP, substantiates the notion of rapid SP metabolism and rapid and selective transport in the brain stem.

Although central injection is normally employed to study the motor behaviors of GR73632 (Rupniak et al., 1997; Steinburg et al., 303 J. Pharmacol. Exp. Ther. 1180-88 (2002)), intraperitoneal administraion can also induce foot tapping in gerbils. Rupniak et al., 1997. Other SP analogs (e.g., DiMe-C7) also produce similar motor behaviors when administered centrally or systemically in rodents (Hasenohrl et al., 1990; 1992).

In the present embodiments, in addition to emesis, systemic administration of GR73632 in the Least Shrew concomitantly produced scratching behavior in a dose-dependent manner, which supports the entry of this agent into deeper brain regions. Indeed, scratchings can be induced in both rodents and shrews via the stimulation of central NK₁- or serotonergic 5-HT2A-receptors. Darmani & Pandya, 107 J. Neurol. Transm. 931-45 (2000); Darmani et al., 1994; Fasmer & Post, 22 Neuropharmacol. 1397-1400 (1983). The failure of SP to induce scratching is likely due to its inability to gain access into the deeper brain regions (e.g., the frontal cortex) responsible for induction of the behavior. This notion is supported by the present findings, as the measured basal SP concentration in shrew frontal cortex actually decreased rather than increased, indicating exogenous SP did not reach this site. SP is not only in intimate association with serotonin in EC cells in the GIT, it is also localized in the dorsal raphe (DR) serotonergic neurons of several species. DR cell bodies express inhibitory serotonergic 5-HT1A-receptors and tachykinergic NK₁-receptors. Rupniak, 2002. It is possible that the high levels of exogenous SP in the brain stem activated dorsal raphe cell body inhibitory NK₁ receptors and thus reduced endogenous SP levels in the frontal cortex via a negative feedback mechanism similar to that described for serotonin. Gobbi & Blier, 26 Peptides 1381-93 (2005).

SP appears to induce vomiting via the activation of NK₁ receptor because its emetic activity was sensitive to the selective non-peptide NK₁ receptor antagonist, CP99,994. SP may induce emesis indirectly through other systems at high doses, however, as evidenced by the variety of emetic challenges against which NK₁ receptor antagonists have shown anti-emetic activity. Andrews & Rudd, 2004. Thus, additional pharmacological experiments were performed using more selective NK₁ receptor agonists (GR73632, ASMSP and SarMet-SP) and antagonists (e.g., CP99,994 and L733060) as well as selective NK₂ and NK₃ receptor selective agonists (such as GR64349 and Pro7-neurokinin B, respectively) and corresponding non-peptide antagonists (GR159897 and SB18795, respectively). The present embodiments confirmed the important role of NK₁ receptor for the induction of both emesis and scratching behavior, because the brain-penetrating NK₁ receptor-selective agonist GR73632 caused maximal emesis and scratching at 5 mg/kg, while the NK₂ and NK₃ receptor agonists were without effect at 10 mg/kg. Moreover, only selective NK₁ receptor antagonists, CP99,994 and L733060, fully prevented GR73632-induced emesis (10 mg/kg to 20 mg/kg) in a dose dependent fashion, while a 20 mg/kg dose of the selective NK₂ and NK₃ receptor non-peptide antagonists failed to affect the induced vomiting.

One of the general characteristics of the antagonism of diverse emetic stimuli by NK₁ receptor blockers is that the latency to first vomit is not usually affected until vomiting is more than 80% inhibited (Andrews & Rudd, 2004) Likewise, in the present work, the latency of GR73632-induced emesis was not affected by either NK₁ receptor antagonist in a dose-dependent manner. The anti-emetic effects of some NK₁ receptor antagonists (e.g., GR205171 and CP99,994) have been confirmed already against other emetogens (motion, CIS, nicotine and halothane) in a larger species of shrew, Suncus murinus (House Musk Shrew). Gardener & Perren, 37 Neuropharmacol. 1643-44 (1998); Tattersall et al., 34 Neuropharmicol. 1607-99 (1995). These antagonists prevented the induced emesis in S. murinus at doses similar to those used in the present study, although CP99,994 is ineffective against vomiting produced by the mechanical stimulation of the upper GIT of S. murinus. Andrews et al., 1996.

Although both of the tested NK₁ receptor antagonists were fully effective against emesis in the Least Shrew, these agents failed to completely prevent GR73632-induced scratchings. CP99,994 tended to attenuate the scratching frequency but the reduction failed to attain significance, while L733060's reduction did attain significance. Several factors can account for the anomaly: First, scratching is a highly variable behavior and the large variance in the control group would influence the statistical outcome. Second, the duration of action of CP99,994 is short since it undergoes both extensive first-pass metabolism in the liver (Ward et al., 8th RSC-SCI Medicinal Chem. Symp., Cambridge, U.K (1995)), and a rapid efflux from the brain (Rupniak et al., 1997). Indeed, CP99,994 can potently and significantly prevent foot tapping in gerbils when administered intravenously just before central injection of GR73632, but not when administered orally one hour prior to central injection of the agonist. Rupniak et al., 1997. Third, larger doses of another NK₁ receptor antagonist (PD154075) are required to prevent foot tappings in gerbils relative to its efficacy in preventing CIS-induced emesis in the ferret. Singh et al., 1997. Finally, the NK₁ receptor antagonist RPR100893 was effective against CIS-induced emesis in ferrets, but failed to prevent GR73632-induced foot tappings in gerbils. Rupniak et al., 1997. Neither of the tested NK₂ or NK₃ receptor agonists induced scratching behavior, nor did their selective non-peptide antagonists modify GR73632-induced scratching. Thus, the present invention affirms a direct role of NK₁ receptors in SP-induced emesis, and further confirms the induced scratching behavior is also mediated by NK₁ receptor activation in the Least Shrew.

Although the effect of an NK₁ receptor agonist on Fos-IR activity in the brain stem DVC nuclei of an emetic species has not yet been reported, several studies have demonstrated that other centrally/peripherally-acting emetic stimuli induce strong Fos-expression in neurons in the medial subnucleus of the NTS and less robustly but significantly in the DMNX of diverse vomiting species including the House Musk Shrew (Andrews et al., 2000; Biossonade et al., 266 Am. J. Physiol. R118-26 (1994); Biossonade & Davision, 271 Am. J. Physiol. R228-36 (1996); Reynolds et al., 565 Brain Res. 231-36 (1991); Miller & Ruggiero, 14 J. Neurosci. 871-88 (1994); Ito et al., 107 Autonomic Neurosci. Basic Clin. 1-8 (2002); Ito et al., 51 Exp. Anim. 19-25 (2003); Van Sickle et al., 285 Gastroint. Liver Physiol. G566-76 (2003); Zaman et al., 39 Neuropharmacol. 316-23 (2000)).

In line with these studies, analysis of Fos-IR for neuronal activation demonstrated that systemic administration of an emetic dose of the NK₁ receptor agonist GR73632, also produced a similar pattern of vigorous Fos-IR expression in the medial subnucleus of the NTS, and less robustly but significantly in the DMNX of the Least Shrew. The NTS as a whole is not functionally related to emesis and in this species, the entire NTS is contained within a length of approximately 240 μm (Ray & Darmani, 1156 Brain Res. 99-111 (2007)). Thus, in the Least Shrew coronal sections, only rarely was more than one section found to have the medial portion of the NTS. The differences in Fos-IR between vomiting and nonvomiting control groups were robust and significant, however. The discussed studies also indicate that while some emetic stimuli (e.g., irridiation, veratrine, CIS, resiniferatoxin, intraduodenal hypertonic saline) enhance Fos-expression in the AP, others do not (e.g., motion, loperamide and electrical stimulation of the vagus). In the present study, GR73632 had no significant effect on Fos-IR activity in the AP of the Least Shrew. In contrast, i.c.v. administration of the NK₁ receptor selective agonist Sar Met-SP has been shown to activate Fos-expression in both the NTS and AP of the non-vomiting guinea-pig. Yip & Chahl, 1999. The results of the Fos-IR activity presented herein are consistent with the distribution of NK1 receptors in the dorsal brain stem and with other functional studies of NK₁ receptor activity (Andrews & Rudd, 2004; Satake & Kawada, 7 Drug Targets, 962-74 (2006); Yip & Chahl, 1999 and 2000). Indeed, NK₁ receptor expression and Fos-IR activity (following emetic stimuli) are most dense in the NTS, although there are lesser amounts present in other portions of the DVC. The lack of stimulation of the AP suggests that GR736332 is acting directly in the DVC (NTS and DMNX) to induce vomiting.

The GIT can be another potential anatomical substrate via which GR73632 could modulate emesis since NK₁ receptors are present on the vagal afferents, in the enteric nervous system (ENS) and in intestinal tissue (Holzer & Holzer-Petche, 73 Pharmacol. Ther., 173-217 (1997a); Holzer & Holzer-Petche 73 Pharmacol. Ther., 219-61 (1997b); lino et al, 556 J. Physiol. 521-30 (2004); Harrington et al., 17 Neurogastroenterol. Motl. 727-37 (2005); Andrews & Rudd, 2004)), where they may directly or indirectly stimulate intestinal motility (Bornstein et al., 16 Neurogastroenterol. Motil. 34-38 (2004); El-Mahmoudi et al., 73 Life Sci. 1939-51 (2003); Holzer & Holzer-Petsche, 1997a; Holzer & Holzer-Petsche, 1997b)). Indeed, Fos-IR was frequently present in the Least Shrew ENS whether or not vomiting occurred. In shrews that vomited, however, a modest but significant increase in Fos-IR in the ENS was found. These and the existing peripheral findings, combined with the ability of SP to relax the lower esophageal sphincter (an event that occurs during emesis) via NK₁ receptors (Smid et al., 10 Neurogastrolcentrol. Motil. 149-56 (1998)), and to generate retroperistalsis (Niel, Assoc. des physiologists, A65-76 (September 1991)), are compelling evidence for a serious consideration of peripheral involvement of NK₁ receptors in vomiting.

In the present invention, intraperitoneally-administered SSP-saporin was used to helped clarify the degree to which the enteric gastrointestinal NK₁ receptor system contribute to emesis. Previous studies have shown both the effectiveness and specificity of this immunotoxin in other in vivo models, lesioning NK₁-containing neurons to affect behaviors including pain modulation, chemoreception, and ejaculatory control (Wiley et al., 146 Neurosci. 1333-45 (2007); Nattie & Li, 101 J. Appl. Physioll. 1596-1606 (2006); Truitt & Coolen, 197 Sci. 1566-69 (2002)). These studies used either intraparenchymal or intrathecal injections of SSP-saporin, however. SSP-saporin was also used in the current study because the sarcosine and dioxymethionine modifications render the SP molecule much more resistant to peptidases, a condition necessary to allow the immunotoxin to diffuse through enough of the enteric nervous system to produce a significant lesion, while still preventing access into the brain.

Indeed, the present results confirm that peripherally-administered SSP-saporin does not penetrate the blood brain barrier, even via the area postrema, as evidenced by the completely normal immunoreactivity for both NK₁ receptor and SP in the DVC. The 1.2 mg/kg dose used is a relatively low dose, but large enough to eliminate NK₁ receptor-IR within the 2 cm length of small intestine (mostly jejunum) harvested at perfusion. After four days, even SP-IR fibers innervating the intestinal wall had shown alterations, presumably due to loss of target cells. Four days after SSP-saporin treatment, the emetic and scratching dose-response effects of GR73632 were investigated exactly per the initial dose response study in drug-naive shrews.

Peripheral NK₁ receptor ablation caused profound quantitative and qualitative changes in the ability of GR73632 to induce emesis. Indeed, in addition to a rightward shift in the number of NK₁ receptor-ablated shrews vomiting in response to varying doses of the NK₁ receptor agonist, these NK₁ receptor-ablated animals also exhibited significantly smaller mean frequencies of vomits. In addition, although the largest tested dose (5 mg/kg) did induce emesis in all ablated shrews, these animals were unable to execute each vomit rapidly. Rather, the few rhythmic abdominal muscular retching movements with corresponding ondulatory mouth openings (which normally required 2-4 second to expel the vomit in response to GR73632 administration in naive shrews), required 15-30 second for the completion of each ejection, as though these animals were unable to generate a significant retroperistaltic intestinal movement (Niel, Assoc.des physiologists, A65-76 (September 1991)), to sweep up the contents of the digestive tract and expel the vomit. Although highly effective in producing peripheral lesion, SSP-saporin injection did not eliminate brain SP-IR, nor did it completely eliminate emesis produced by GR73632. Moreover, the tested doses of GR73632 produced an expected frequency of scratchings in ablated shrews, similar to those in control shrews that had received a single vehicle injection (saline) four days prior to these challenge doses of GR73632. Although the 5 mg/kg dose in ablated shrews tended to cause fewer than the expected mean control frequency of scratchings, the reduction is not (P>0.05) significant, and these animals spent significant time trying to vomit. Thus, the behavioral and histochemical results of SSP-saporin injections provide solid evidence for a mixed central/peripheral activity for SP on the emetic reflex. These findings also indicate that activation of gastrointestinal NK₁ receptors in the GIT is not required for the initiation of the vomiting process, since other tested stable and selective CNS nonpenetrating NK₁ receptor agonists failed to induce significant dose-dependent emesis even at doses larger than 5 mg/kg. Intact gastrointestinal NK₁ receptors and their activation are required to rapidly execute vomit expulsion.

Via the utilization of diverse behavioral, biochemical, and immunohistochemical techniques provided for herein in the context of the Least Shrew model, the present invention provides for (a) the production of emesis via the activation of NK₁ receptors using SP and the brain penetrating tachykinin NK₁ receptor-selective agonist GR73632; (b) a cardinal initiating role for the induction of emesis for central NK₁ receptors present in the DVC, specifically in the medial subnucleus of the NTS and in the DMNX nuclei; and (c) the validation of the Least Shrew as a specific and rapid behavioral model to screen concomitantly both the CNS penetration and the antiemetic potential of tachykinin NK₁ receptor antagonists.

The present invention also provides for methods of treating emesis with leukotriene antagonists, including both CysLT₁ antagonists and inhibitors of leukotriene synthesis, that have demonstrated antiemetic potential and possible clinical utility against emesis produced by a variety of agents including chemotherapeutics, inflammatory agents as well as bacterial toxins and other gastrointestinal conditions. In the absence of a suitable animal model, the discovery of emetic action of leukotrienes LTC₄ and LTD₄ and the role of CysLT₁ receptors were unknown. The present invention provides a model in which the direct emetic effects of systemic leukotriene LTC₄ and LTD₄ and CysLT₁ receptors are systematically characterized in a vomiting species: the Least Shrew model of emesis. More specifically, aspects of the present Least Shrew emetic model demonstrates or suggests that both CysLT₁ antagonists and inhibitors of leukotriene synthesis have antiemetic potential and possible clinical utility against emesis produced by a variety of agents including chemotherapeutics, inflammatory agents as well as bacterial toxins and other gastrointestinal conditions.

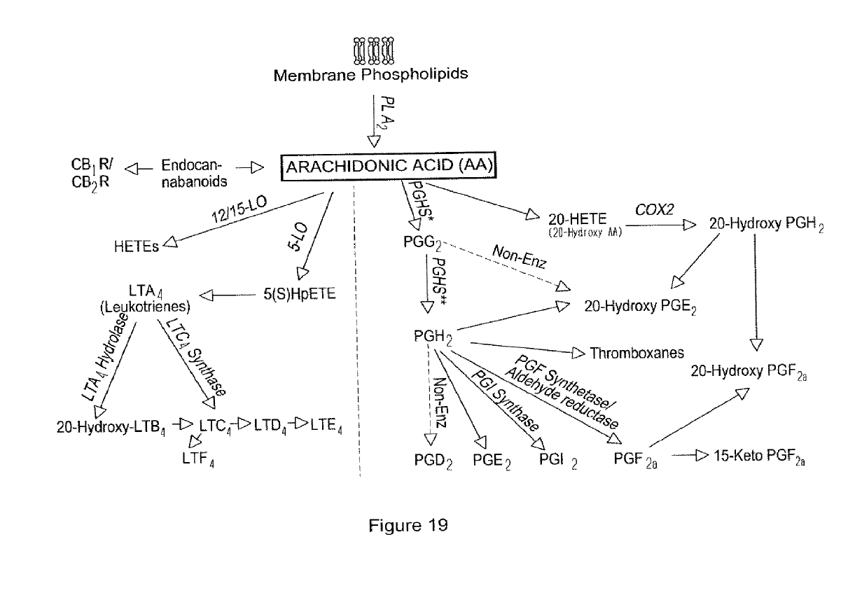

Anti-NK₁ drugs are less effective in the acute phase than the more-common anti-serotonin 5-HT₃ receptor antagonists (like ondansetron). Where the current anti-NK₁ drugs prove valuable is in the delayed phase, where they exhibit good antiemetic effectiveness in clinical trials. CIS increases the synthesis of proinflammatory mediators derived from arachidonic acid such as prostaglandins (PGs) and leukotrienes (FIG. 19). Thus, to better prevent chemotherapy-induced emesis (e.g., CIS-induced emesis), combinations of serotonin 5-HT₃ receptor antagonists and NK₁ receptor antagonists are used in addition to anti-inflammatory drugs such as dexamethasone.

More specifically, arachidonic acid is released from cellular phospholipids by cytosolic phospholipase A_{2α}, which is then oxygenated by distinct enzyme systems such as lipooxygenases (LO's). The 5-LO enzyme cascade produces leukotrienes from arachedonic acid. These lipid signaling molecules play roles in inflammation and immune processes, but some may also play a role in emesis (FIG. 19). Indeed, inhibitors of phospholipase A_{2α} such as dexamethasone are used as adjunct antiemetics for the prevention of acute and delayed chemotherapy-induced vomiting in cancer patients. Inflammatory eicosanoids generated by the 5-LO pathway of arachedonic acid metabolism have at least four receptors: the BLT1- and BLT2-receptors which bind to leukotriene LTB₄; and the cysteinyl leukotriene (CysLT) CysLT₁-receptor and CysLT₂-receptor which have affinity for leukotrienes (LT) LTC₄, LTD₄, LTE₄, and LTF₄.

The present invention provides, for the first time, that some leukotrienes (e.g., LTC₄ and LTD₄), but not all (e.g., LTA₄, LTB₄, LTE₄, and LTF₄), are potent emetogens in the Least Shrew emesis model. More specifically, the cysteinyl leukotrienes (CysLT) LTC₄ and LTD₄ are potent lipid mediators of hypersensitivity and inflammatory conditions. As shown in FIG. 19, cysLTs are products of arachidonic acid metabolism. They induce bronchoconstriction, increase microvascular permeability, and are vasoconstrictors of coronary arteries. Their biological effects are transduced by a pair of G protein-coupled receptors for CysLT₁ and CysLT₂. Lynch et al., 399 Nature, 789093 (1999); Heise et al., 275 J. Biol. Chem. 30531-36 (2000); Sarau et al., 56 Mol. Pharmacol. 657-63 (1999). Hence, two approaches to anti-leukotriene therapy have been developed: blocking their production by inhibiting the action of 5-lipoxygenase enzyme, or blocking the LT receptors (CysLTR₁).

Pranlucast (4-Oxo-8-[(4-phenylbutoxy)benzoylamino]-2-(tetrazol-5-yl)-4H-1-benzopyran) (pranlukast, ONO-1078, Ono Pharma USA, Inc., Lawrenceville, N.J.) is a compound having potential antagonistic action against leucotriene LTC₄ and LTD₄ is a potent, selective, and orally active CysLT₁ receptor antagonist. Taniguchi et al., 92 J. Allergy Clin. Immunol. 507-12 (1993); U.S. Pat. No. 5,876,760. Pranlukast may be depicted as follows:

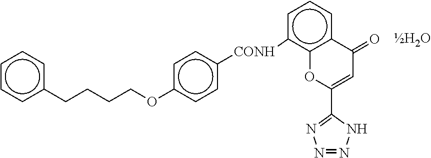

The CysLT₁ and CysLT₂ receptors are distinguished by their sensitivity to CysLT receptor antagonists, pranlucast (and related antagonists, CysLT₁ specific) or the leukotriene analog Bay u9773 ((6(R)-(4′-carboxyphenylthio)-5(S)-hydroxy-7(E),9(E), 11(Z), 14(Z)-eicosatetraenoic acid)), (a mixed CysLT_{1/2} antagonist and CysLT₂ partial agonist). Leukotriene-induced emesis appears to be specifically related to activation of CysLT₁ receptors because, as shown herein, specific blockers (e.g., pranlucast or zafirlucast) of these receptors prevent LTC₄-induced emesis completely and in a dose-dependent manner. Furthermore, leukotrienes LTA₄ (the metabolic precursor of LTC₄/D₄) and LTB₄, which do not bind to the CysLT₁ or CysLT₂ receptors, were non-emetic. Because the current combination of antiemetic drugs are only effective in 60%-80% of patients receiving chemotherapy, utilization of CysLT₁ receptor antagonists (or inhibitors of enzymes responsible for leukotriene production, e.g., MK-866 or zileuton) as antiemetics, either alone or in combination with other antiemetics, enhances the armamentarium not only against chemotherapy-induced nausea and vomiting but also against gastrointestinal and inflammatory conditions that lead to emesis.

That cysLT₁ antagonists are already used in the clinic for the prevention of asthma lends support to their safe use in humans. Pranlucast, trade named Ultair™ and Onon™, was the first cysteinyl (peptidyl) leukotriene receptor antagonist (LTRA) marketed for the treatment of asthma. Barnes et al., 111 Chest, 52-60 (1997). Clinical studies in Japan, Europe, and North America showed that pranlucast significantly attenuated bronchoconstriction in response to a variety of allergen challenges as well as to inhaled LTD₄. Id. Pranlucast binds to the human CysLT₁ and CysLT₂ receptors with IC₅₀ values of approximately 4-7 nM and 3,600 nM, respectively. Lynch et al., 1999; Heise et al., 2000; Sarau et al., 1999. Other leukotriene-antagonists include zafirlukast (a cysLT₁RA sold as Accolate®, AstraZeneca, London, UK), montelukast (a cysLT₁RA sold as Singulair®, Merck & Co., Inc., Whitehouse Station, N.J.), and antibodies that bind the LTC₄ receptor (see, e.g., U.S. Patent application Pub. No. 20050037968).

As noted above, leukotriene activity may also be mediated through inhibition of the synthetic pathway of leukotriene metabolism. Drugs for this approach include zileuton, a synthetic derivative of hydroxyurea that blocks 5-lipoxygenase (Zyflo®, Critical Therapeutics, Inc., Lexington, Mass.); and drugs such as MK-886, that blocks the 5-lipoxygenase activating protein. Other leukotriene synthesis inhibitors include BAY x 1005 and BAY y 1015 (Bayer Corp., New Haven, Conn.). BAY u9773 (Bayer UK, Ltd., Slough, UK) is an example of a LTC₄ antagonist. Additional cysLT₁, LTD₄, and LTC₄ antagonists may be identified by three-dimensional modeling studies. See, e.g., Zwaagstra et al., 41(9) J. Med. Chem. 1439-45 (1998). Hence, the invention is not limited to a particular compound, per se. Indeed, the present invention also encompasses derivatives, prodrugs, and metabolites of leukotriene antagonists with antiemetic potential.

Leukotriene antagonists identified as antiemetic in the Least Shrew model, according to the present invention, may also be tested in combination with other antiemetic agents and/or anti-inflammatory agents. More specifically, current antiemetics are often grouped based on their function. The major groups are 5-hydroxy-tryptamine (serotonin) 3 (5-HT₃) receptor antagonists, NK₁ receptor antagonists, corticosteroids, dopamine receptor antagonists, and cannabinoids.

Briefly, the 5-HT₃ receptor is a serotonin receptor, part of seven classes of serotonergic family receptors. This family of receptors is important for many aspects in neuronal signaling. The 5-HT₃ antagonists commonly used include dolasetron (commercially Anzemet®, Sanofi-Aventis, U.S. LLC, Bridgewater, N.J.), granisetron (Kytril®, Roche Labs., Nutley, N.J.), ondansetron (Zofran®, GlaxoSmithKline plc, Philadelphia, Pa.), tropisetron (Navoban®, Novartis Int'l AG, Basel, Switzerland), and palonosetron (Aloxi®, MGI Pharma, Inc., Bloomington, Minn.). These compounds effectively block the normal signaling through the 5-HT₃ receptor, inhibiting the signal that would normally reach the vomiting center.

Neurokinin type 1 (NK₁) receptor is found in the vomiting center and in the abdominal vagus (a nerve running along the esophagus and the stomach) and binds the neuropeptide called substance P. Binding of substance P elicits a neuronal signal that ultimately leads to vomiting and nausea. Aprepitant (Emend®, Merck & Co., Inc., Whitehouse Station, N.J.) is a chemical compound that belongs to a class of drugs called substance P antagonists (SPA), and mediates its effect by acting on neurokinin 1 receptor. See also Darmani et al., 2008.

Anti-inflammatory drugs, such as corticosteroids, may also be included for testing in the present invention. Corticosteroids are hormones in the steroid class meaning they have the base structure of cholesterol. The corticosteroids have a range of function in the body. Dexamethasone is a synthetic corticosteroid of the glucocorticoid class and is the most commonly prescribed corticosteroid. It is often given in conjunction with 5-HT₃ antagonists to augment their effects.

American Society of Clinical Oncology recommended, in its 2006 Guidelines for Use of Antiemetics, that a three-drug combination of a 5-HT₃ serotonin receptor antagonist, dexamethasone, and aprepitant be used before chemotherapy. In all patients receiving CIS and all other agents of high emetic risk, the three-drug combination of 5-HT₃ receptor antagonist, dexamethasone and aprepitant was recommended. For patients receiving a chemotherapy of moderate emetic risk, a two-drug combination of a 5-HT₃ receptor serotonin antagonist and dexamethasone was recommended. For low risk patients, dexamethasone was recommended.

Additionally, dopamine receptor antagonists inhibit the class of receptors that binds dopamine, a neurotransmitter. Dopamine is an emetic and can induce nausea, hence blocking dopamine D_{2/3} receptors is another antiemetic approach. Domperidone (Motihum®, Janssen-Ortho, Toronto, Canada) and metoclopramide (Baxter Healthcare Corp., Deerfield, Ill.) are two dopamine receptor antagonists used for antiemetic treatment.

Cannabinoids are drugs that bind to cannabinoid receptors (CB) found throughout the central (CB₁) and peripheral (CB₁ and CB₂) nervous systems. There are endogenous cannabinoids produced in humans that bind to these receptors. The synthetic cannabinoids nabilone (Cesamet®, Valeant Pharma. Intl, Aliso Viejo, Calif.) and dronabinol (Marinol®, Solvay Pharma., Inc., Marietta, Ga.) bind to CBs. Cannabinoids are antiemetic in that they are agonists to the CBs and partially block the release of other neurotransmitters.

Finally, other adjunct antiemetic agents may be used in the invention provided herein. For example, lorazepam and diphenhydramine are useful adjuncts to antiemetic drugs, but are not recommended as single agents.

In another embodiment, the antiemetic potential of CysLT₁-receptor antagonists may be evaluated against inflammatory emetogens such as bacterial toxins. For example, cereulide is a cyclic dodecadepsipeptide from a pathogenic bacteria Bacillus cereus, which shows the emetic toxicity, as does valinomycin toxin from Streptomyces tsusimaensis. Staphylococcus aureus also produces emetic toxin. The ability to study these emetogens in conjunction with antiemetics in the Least Shrew model enhances the understanding of the emetic mechanisms of these agents.

The antiemetic potential of the leukotriene antagonists of the present invention, e.g., a CysLT₁ receptor antagonist or an inhibitor of leukotriene synthetic enzymes, may be tested in an animal model by selecting an animal model of emesis, in which the animal species exhibits emesis in response to leukotriene LTC₄ or LTD₄, such as the Least Shrew; dividing test animals of said animal species into at least two groups; administering a potential leukotriene antagonist, such as a CysLT₁ receptor antagonist to one group of animals; administering a leukotriene LTC₄ (or LTD₄) to a non-pretreated group and the CysLT₁ receptor antagonist-pretreated group of animals; observing and comparing indices emesis in said groups. The testing may include other emetogens for comparision, or may include other antiemetics for comparison. For example, administering a potential CysLT₁ receptor in combination with one or more of other classes of antiemetics such as a 5-HT₃ receptor antagonist, an NK₁ receptor antagonist, or an anti-inflammatory agent such as dexamethasone to determine their antiemetic potential and possible additive and/or synergistic antiemetic activity against both phases of chemotherapy-induced vomiting. Or, for example, administering a potential leukotriene synthesis inhibitor either alone or in combination with one or more of other classes of antiemetics to determine their antiemetic potential and possible additive and/or synergistic antiemetic activity against both phases of chemotherapy-induced vomiting.

The instant invention also relates to methods of alleviating emesis by treating subjects in need thereof with at least one leukotriene antagonist. As noted previously herein, several leukotriene antagonists are already used in humans, e.g., for treating asthma and allergies, and several have been used for over a decade. See, e.g., Drazen et al., 340(3) Drug Therapy 197-06 (1999). These medicaments are prepared in several oral formulations, which are well known in the art. Any necessary modifications to these known formulations for use in antiemetics are well within the grasp of those of ordinary skill in light of the present invention. Additionally, the appropriate route of administration, dose, and dosing schedule may be determined by the skilled practitioner in light of the particular patient needs. Thus, for example, the practitioner may determine whether the leukotriene antagonist should be administered before, with, or following exposure to an emetic agent, e.g., CIS.

Another embodiment of the present invention demonstrates the utility of the Least Shrew model in studying whether 5-HT₃ receptor antagonists (e.g., tropisetron) combined with dexamethasone are effective for the acute phase of CIS-induced emesis. This study determined the possible additive or synergistic antiemetic efficacy of Δ⁹-tetrahydrocannabinol (Δ⁹-THC) when combined with tropisetron or dexamethasone (DEX). Δ⁹-THC (0-10 mg/kg, i.p.) was injected in combination with tropisetron (0-5 mg/kg, i.p.) or dexamethasone (0-20 mg/kg, i.p.) prior to CIS (20 mg/kg, i.p.) in the Least Shrew, and the induced emesis was recorded for 60 minutes. CIS-induced vomiting was dose-dependently and significantly attenuated by individual administration of Δ⁹-THC (59%-97% reductions) and tropisetron (79%-100% attenuation), but not dexamethasone (26%-40%), although a trend (pb 1) towards reduced vomiting frequency following DEX was noted. Low doses of Δ⁹-THC (0.25 or 0.5 mg/kg) when combined with low doses of tropisetron (0.025, 0.1, or 0.25 mg/kg) were more efficacious in reducing emesis frequency than when given individually, but Δ⁹-THC had no antiemetic interactions with DEX. No tested combination provided a significantly greater effect on the number of animals vomiting than their individually-administered counterparts. The modest interaction of Δ⁹-THC with tropisetron suggests they activate overlapping antiemetic mechanisms, while the lack of interaction with dexamethasone needs further clarification.

To boost the efficacy of either 5-HT₃ or NK₁ antagonist antiemetics, anti-inflammatory steroids such as dexamethasone (DEX) are frequently used in combination with them (Darmani, 2002; Hesketh et al., 2006). Clinical trials have demonstrated the effectiveness of DEX as an adjunct therapy in combination with a number of different antiemetics, in patients receiving a wide range of chemotherapeutic regimens (see Grunberg, 2007). The mechanism of action of DEX is still unknown, however, and results in animal models have been highly variable. DEX administered alone has been found reasonably effective in the acute phase in most, but not all animal species, but only modestly effective in the delayed phase in most animals, and even ineffective in the house musk shrew model (Fukunaka et al., 1998; Malik et al., 2007; Rudd et al., 2000; Sam et al., 2003; Tanihata et al., 2004). Because these model animals had different emetic responses to equivalent doses of CIS, the doses of antiemetics needed in the cited studies varied significantly, thus muddying the question of how much species differences and/or dosing differences contributed to the responses to DEX and the other tested antiemetics.

Research also points to Δ⁹-THC, one of the psychoactive constituents of marijuana, as an antiemetic therapy against CIV. Δ⁹-THC inhibits CIV via stimulation of brainstem, and possibly GIT, CB₁ cannabinoid receptors (Darmani, 2001b; Van Sickle et al., 2003). The exact locus and mechanism of their antiemetic effect are still unclear, but cannabinoid receptors are known to be coupled to G-proteins, and frequently inhibit synaptic transmission when stimulated. Thus, their activation may reduce emetogen-induced stimulation of the DVC (Ray et al., 2009; Van Sickle et al., 2003). Cannabinoids show promise as antiemetics against both phases of CIV in animal models (Darmani, 2001b; Ray et al., 2009; Van Sickle et al., 2003) as well as in limited clinical use (Abrahamov et al., 1995; Darmani, 2005).

Clinical studies suggest that in blocking acute-phase CW, a combination of a 5-HT₃ receptor antagonist and DEX is more efficacious relative to either compound tested alone (Hesketh et al., 2006). Early clinical data regarding Δ⁹-THC were similar to those regarding DEX, in that Δ⁹-THC in combination with other antiemetics (e.g. prochlorperizine) enhances the antiemetic efficacy of treatment relative to either compound administered alone (Garb, 1981; Lane et al., 1990). More recently published data specifically confirm the enhanced antiemetic efficacy of 5-HT₃ receptor antagonists when paired with Δ⁹-THC (Kwiatkowska et al., 2004), although this work was limited by only testing single doses of each compound in combination. Thus, to compare the combined antiemetic efficacy of Δ⁹-THC with a 5-HT₃ antagonist or DEX, we studied the dose-response efficacy (whether additive, synergistic, or ineffective) of Δ⁹-THC with either the 5-HT₃ receptor antagonist tropisetron, or the anti-inflammatory steroid DEX, on the acute phase of CIS-induced emesis in the Least Shrew model. The emetic responses of this species to CIS (acute and delayed phases), Δ⁹-THC, tropisetron, and DEX are very similar to those seen in humans (Darmani, 2001a, 1998; Darmani et al., 2005; Ray et al., 2009).

As noted above, CIS is clinically effective against a variety of tumors (Rosenberg, 1985), but is also one of the most emetogenic antitumor therapies currently in use (Laszlo et al., 1985). Because the discomfort from excessive vomiting can result in chemotherapy patient drop-out or poor antitumor therapy outcomes, adjunct antiemetic therapy is almost a necessity. Thus, it is important to understand the mechanisms behind CIV, both to improve the efficacy of the next generation of antiemetics and to indirectly improve antitumor therapy outcomes as well. The different classes of antiemetics currently in use, including the serotonin 5-HT₃ receptor antagonists, anti-inflammatory steroids, and the cannabinoid CB_{'} receptor agonists, have been studied extensively and found to be varyingly effective, either alone or in combination (Bountra et al., 1996; Cubeddu et al., 1992; Darmani, 2002; Hesketh et al., 2006; Kudo et al., 2001; Kwiatkowska et al., 2004; Lane et al., 1990). Indeed, a significant body of literature has described several distinct combinations which are superior in terms of antiemetic potency: (a) a 5-HT₃ receptor antagonist (e.g., tropisetron or ondansetron) and DEX; (b) Δ⁹-THC with dopamine D2 receptor antagonists; or (c) Δ⁹-THC with a 5-HT₃ receptor antagonist (Garb, 1981; Hesketh et al., 2006; Kwiatkowska et al., 2004; Lane et al., 1990). Although they are mostly effective against the acute phase of CIV, none have proven very effective against the delayed phase. Progress has also been hampered by the limited variety of doses tested: in most cases only a single dose of either compound was tested in combination. The work presented herein more thoroughly describes the differences in, and the nature of (additive or synergistic), the enhanced antiemetic efficacy (if any) produced by combinations of different classes of antiemetics. Combinations which include Δ⁹-THC, in part because combination therapies including Δ⁹-THC seem to be clinically effective, but are poorly understood mechanistically.

This embodiment addresses the 5-HT₃ antagonist tropisetron, the CB₁ agonist Δ⁹-THC, and the anti-inflammatory DEX, and their potential antiemetic interactions. DEX is a synthetic steroid related to cortisol, and is used to treat many inflammatory and autoimmune conditions. It is also given to cancer patients to counteract CIV and other side effects. Clinical trials have found that DEX is effective as monotherapy and in combination with other antiemetic agents as a relatively broad-spectrum antiemetic (Ahn et al., 1994; Grunberg, 2007; Malik et al., 2007). In the present work, DEX administration alone (0.25-20 mg/kg) failed to either reduce the frequency of vomiting, however, or to protect shrews from CIS-induced vomiting.

The mechanism and locus of activity of DEX-induced antiemetic activity are still unclear, making the analysis of this anomaly difficult. One possible cause of this anomaly is the species differences between humans and shrews. Clinically, DEX and a 5-HT₃ antagonist together have demonstrated the ability to block the vast majority of acute vomiting due CIS (Drechsler et al., 5 Support Case Cancer, 387-95 (1997); Sorbe et al., 30A Eur. J. Cancer 629-34 (1994)), but were ineffective even combination when administered to the house musk shrew (Suncus murinus; Sam et al., 2003). In contrast to Suncus, however, the data related to emesis in the Least Shrew have been very similar to those collected for humans (see e.g., Darmani, 1998; Darmani et al., 2008; Darmani et al., 1999; Ray et al., 2009), and previously published single-dose data regarding DEX administered alone have indeed shown reduced emetic activity (Darmani et al., 2005). A more likely explanation relates to the relatively high dose CIS used in this study (20 mg/kg). DEX, like other antiemetics, is not 100% effective against CIV in humans, and the dosing regimen of CIS in chemotherapy is typically a lower dose, infused over a longer time period (Hesketh et al., 39 Eur. J. Cancer 1074-80 (2003b); Vera et al., 126-127 Auton Neurosci. 81-92 (2006)). The high dose and intraperitoneal route of administration of CIS used here, which together can produce a very short infusion time, rapid systemic absorption, and a high initial spike in concentration, could have created a situation where the antiemetic ability of DEX was effectively overwhelmed. This would be especially true for the acute phase of CIV, due to its temporal proximity to the injection of CIS. The large dose of CIS was necessary to prevent a potential floor effect, and 10 mg/kg CIS is sufficient to produce emesis the vast majority of least shrews, at this dose it was entirely possible that either drug administered alone could reduce vomiting to the extent that an enhanced interaction would be masked by the efficacy of the first drug. Thus, an attempt was made to strike a balance between the dose of CIS and obtaining a measurable interaction for the drug combinations.

Another key compound in this study, Δ⁹-THC appears to possess antiemetic efficacy in both cancer patients receiving chemotherapy, and in animal models of CIV, an effect most likely mediated by presynaptic inhibition of vagal afferents (or other terminals) via cannabinoid CB₁ receptors (Darmani & Crim, 80 Pharmacol. Biochem. Behay. 35-44 (2005); Darmani & Johnson, 488 Eur. J. Pharmacol. 201-12 (2004); Feigenbaum et al., 169 Eur. J. Pharmacol. 159-65 (1989); Kwiatkowska et al., 174 Psychopharmacol. (Berl) 254-59 (2004); Van Sickle et al., 285 Am. J. Gastrointete. Liver Physiol. G566-76 (2003)). Recent evidence suggests that some cannabinoids can also bind to vanilloid TRPV1 receptors (Coutts & Izzo, 4 Curr. Opin. Pharmacil. 572-79 (2004); Sharkey et al., 25 Eur. J. Neurosci. 2773-83 (2007)), and certain endocannabinoids (e.g. ,2-arachidonoylglycerol, 2-AG) produce constituents of the arachidonic acid cascade (e.g., prostaglandins), activating inflammatory pathways and inducing emesis (Coutts & Izzo, 2004; Cross-Mellor et al., 190 Psychopharmacol. (Berl) 135-43 (2007); Darmani, in BRAIN & BODY MARIJUANA & BEYOND (CRC, Boca Raton, 2005). Thus, these two mechanisms may also play modulatory roles in the regulation of emesis by cannabinoids despite their contradictory natures. In the present study, Δ⁹-THC by itself attenuated both the vomiting frequency and the percentage of shrews vomiting, in a dose-dependent manner. Furthermore, low doses of Δ⁹-THC (0.25 and 0.5 mg/kg) in combination with low doses of tropisetron (0.025 and 0.1 mg/kg) demonstrated greater efficacy than their individually-administered counterparts in reducing the frequency of vomiting. These data are in agreement with published animal studies (Kwiatkowska et al., 2004) and clinical findings (Hesketh et al., 2003a; Meiri et al., 23 Curr. Res. Opin. 533-43 (2007); Slatkin, 5 J. Support. Onocl. 1-9 (2007); Triozzi & Laszlo, 34 Drugs 136-49 (1987)).

The lack of a significant synergistic antiemetic action is not surprising. There are likely overlaps in the mechanisms with which these drugs block emesis, preventing the enhanced antiemetic effect. For example, the mechanism of CB₁ receptor antiemetic agonists, as stated above, likely relies on presynaptic inhibition. This CB₁-mediated inhibition (e.g., in the DVC or GI nerve plexi) could reduce activity generated by postsynaptic, tropisetron-sensitive 5-HT₃ receptor-containing neurons, or by presynaptic terminals which might colocalize these 5-HT₃ receptors (Fink & Gothert, 2007). In fact, there is also evidence that cannabinoids can directly modulate 5-HT₃ receptors allosterically (Barann et al., 137 Br. J. Pharmacol. 589-96 (2002); Xiong et al., Mol. Pharmacol., 2007). If this direct crosstalk is also part of the mechanism of cannabinoid-mediated antiemesis, any potential additive effect may be dampened by interference from 5-HT₃ antagonist binding. The slight enhancement of antiemetic ability by low doses of Δ⁹-THC in combination with low doses of tropisetron would result from incomplete receptor occupancy by either or both drugs, or possibly by incomplete anatomical overlap of cannabinoid and 5-HT₃ receptors. In the case of DEX, emetic behavior would be mediated “downstream” from the presynaptic events modulated by CB₁ receptors. Postsynaptic second-messenger systems, including the prostanoid producing arachidonic acid metabolic pathways, would provide an interface through which DEX- and cannabinoid-mediated systems would overlap. The net effect in this case would be cannabinoid mediated inhibition, or lack of stimulation, of neurons whose down stream antiemetic effector mechanisms were already inhibited, pre-venting the proposed enhancement of antiemetic activity by the combined drug regimen. On the other hand, the mechanisms through which tropisetron and DEX inhibit activation of emesis-initiating (or propagating) neurons would be less likely to interact directly with each other. The putative interactions among the substrates for serotonergic, cannabinergic, and inflammatory signaling are also relevant to the clinic, in that they provide a possible explanation for the relatively greater clinical effectiveness of 5-HT3 antagonists when paired with DEX (Ahn et al., 17 Am. J. Clin. Oncol., 150-56 (1994); Eberhart et al., 36 Eur. J. Clin. Investig. 580-87 (2006)), vs. the much less impressive results pairing Δ⁹-THC and 5-HT₃ antagonists (Meiri et al., 2007; Slatkin, 2007). Further studies will more thoroughly dissect the anatomical and pharmacological substrates of emesis, because the underlying mechanisms of the emetic reflex are still unclear, and this understanding will help the development of more effective (i.e., broader spectrum and/or biphasic activity) antiemetics.

Another embodiment of the present invention relates to a Least Shrew model for examining the substrates of cannabinoid-mediated inhibition of both of the CIV-induced emetic phases via immunolabeling for serotonin, SP, cannabinoid receptors 1 and 2 (CB₁, CB₂), and the neuronal activation marker Fos. Briefly, shrews were injected with CIS (10 mg/kg, i.p.), and one of vehicle, Δ9-THC, or both Δ9-THC and the CB_{'} receptor antagonist SR141716A (2 mg/kg i.p.), and monitored for vomiting. Δ9-THC-pretreatment caused concurrent decreases in the number of shrews expressing vomiting and Fos-immunoreactivity (Fos-IR), effects which were blocked by SR141716A-pretreatment. Acute phase vomiting induced Fos-IR in the solitary tract nucleus (NTS), dorsal motor nucleus of the vagus (DMNX), and area postrema (AP), whereas in the delayed phase Fos-IR was not induced in the AP at all, and was induced at lower levels in the other nuclei when compared to the acute phase. CB₁ receptor-IR in the NTS was dense, punctate labeling indicative of presynaptic elements, which surrounded Fos-expressing NTS neurons. CB₂ receptor-IR was not found in neuronal elements, but in vascular-appearing structures. All areas correlated with serotonin-IR and Substance P-IR. These results support published acute phase data in other species, and are the first describing Fos-IR following delayed phase emesis. The data suggest overlapping but separate mechanisms are invoked for each phase, which are sensitive to antiemetic effects of Δ⁹-THC mediated by CB₁ receptors.

As discussed previously, mechanistically, CIV involves a substantial release of serotonin (5-HT) by CIS in the gastrointestinal tract (GIT), and probably in the brain as well, which initiates a reflex arc via vagal 5-HT₃ receptors activating the dorsal vagal complex (DVC) of the central nervous system (CNS) (Endo et al., 153 Toxicology 189-201; Hesketh et al., 39 Eur. J. Cancer 1074-80 (2003); Higa et al., 12 J. Oncol. Pharm. Pract. 201-09 (2006)). The DVC is a cluster of medullary nuclei which performs several emetic functions: The area postrema (AP), a circumventricular organ, allows bloodborne emetogenic chemicals access to the brain. The dorsal motor nucleus of the vagus (DMNX) may receive vagal afferents from the nodose ganglion (Leslie et al., 38 Neurosci. 667-73 (1990)), but is primarily innervated by the medial solitary tract nucleus (NTS). It sends efferents to the GIT and to a central pattern generator which modulates emetic retroperistaltic activity. The medial solitary tract nucleus (NTS) is a key site for integrating diverse emesis-related afferents from the vagus and a wide range of brain areas (see Hornby, 111(8A) Am. J. Med. 106S-12S (2001)).

The 5-HT₃ receptor antagonists (e.g., ondansetron) are effective antiemetics against the acute phase of CIV (Darmani, 105 J. Neural. Transm. 1143-54 (1998); Kwiatkowska et al., 174 Psychopharmacology (Berl) 254-59 (2004)), but not against the delayed phase (Bountra et al., 53(S1) Oncology 102-09 (1996)). Research has implicated neurokinin NK₁ receptor activation by Substance P (SP) as one putative mediator of the delayed phase (Bountra et al., 1996; Tanihata et al., 461 Eur. J. Pharmacol. 197-206 (2003)). In fact, SP itself is emetic, and both NK₁ antagonists (Darmani et al., 1214 Brain Res. 58-72 (2008)), and Δ⁹-THC (in P56 15TH ANN. SYMPOSIUM CANNABINOIDS (2005)), can block SP-induced vomiting. Whereas animal studies demonstrate potent antiemetic activity of NK1 receptor antagonists against both phases of CIV (Rudd et al., 366 Eur. J. Pharmacol. 243-52 (1999); Tanihata et al., 2003)), clinical studies have been disappointing. NK1 antagonists alone are ineffective against either phase of CIV (Hesketh et al., 39 Eur. J. Cancer 1074-80 (2003); Rubenstein et al., 12 Cancer J. 341-47 (2006)), but potentiate antiemetic efficacy when combined with standard antiemetics (Hesketh et al., 21 J. Clin. Oncol. 4112-19 (2003)).

Cannabinoid neurotransmission, mediated by two G-protein coupled receptors (CB₁ or CB₂), can also modulate vomiting. In animals, THC, synthetic CB₁ receptor agonists, and related ligands are potent, broad-spectrum antiemetics (Darmani & Johnson, 488 Eur. J. Pharmacol. 201-12 (2004); Simoneeau, 94 Anesthesiology 882-87 (2001); Van Sickle et al., 285 Am. J. Physiol. Gastrointest Liver Physiol. G566-76 (2003)), whose activity may also include interactions with 5-HT₃ (Kwiatowski et al., 2004) or TRPV1 vanilloid (Sharkey et al., 25 Eur. J. Neurosci. 2773-82 (2007)) receptors, while the CB₂ receptor does not appear emetically relevant (Darmani, 123 Methods Mol. Med. 169-89 (2006); Simoneau et al., 94 Anesthesiology 882-87 (2001)). In limited clinical use, cannabinoids appeared antiemetic against both phases of CIV (Abrahamov et al., 56 Life Sci. 2097-102 (1995); Darmani, 2005). CB₁ antagonists reverse the antiemetic effects of THC (Darmani & Johnson, 2004; Van Sickle et al., 2003), but require high doses to be emetogenic themselves (Darmani & Johnson, 2004; Simoneau et al., 2001). The antivomiting effect of exogenous cannabinoids appears improved when combined with standard antiemetic drugs (Darmani, 2005; Slatkin, 5 J. Support Oncol. 1-9 (2007)).

Previously, mechanistic aspects of vomiting and the antiemetic action of cannabinoids remain unclear. Emesis-related functional studies have been limited by only examining the acute phase (Miller & Ruggiera, 14 J. Neurosci. 871-88 (1994)), or using non-vomiting species (Horn et al., 132(1-2) Auton Neurosci. 44-51 (2006); Vera et al., 126-127 Auton Neurosci. 81-92 (2006)). CNS cannabinoid receptors are widespread and multifunctional (Haring et al., 146 Neurosci. 1212-19 (2007)), complicating study of the anatomical substrates of their antiemetic activity. Furthermore, the exact locus (or loci) of effect of antiemetic cannabinoids has not been clearly identified, although CB₁receptors on vagal afferents to the DVC are implicated (Van Sickle et al., 2003). Finally, data are lacking on the relationship of cannabinoid receptors to components of other emetogenic neurotransmitter systems (e.g., 5-HT or SP).

The present invention allows for the elucidation of the neural substrates mediating CIV and THC-related antiemesis in both phases. The approach used immunohistochemistry (IHC) for Fos protein (Fos) to indicate neuronal activation, alone and in combination with IHC for CB₁ and CB₂ receptors, SP, and 5-HT, to study the DVC anatomically and neurochemically during both phases of CIV in the Least Shrew, C. parva. This mammal is emesis-competent, with well-defined acute (0-3 hour post-CIS exposure, peaking at 2 hour) and delayed (28-35 hours post-CIS, peaking at 33 hour) emetic phases, and emetic responses similar to those of humans (Darmani, 105 J. Neural Transm 1143-54 (1998); Darmani, in BRAIN & BODY'S MARIJUANA & BEYOND, 393-418 (Onaivi et al., eds., CRC, Boca Raton, 2005); Darmani et al., 49 Neuropharmacol. 502-13 (2005); Ray & Darmani, Program #739 Soc'y Neurosci. Ann. Meeting 16 (2006)).

The Least Shrew model allows for the first examination of functional anatomy to study the effect of THC on the delayed phase of CIV in a vomiting animal model. It confirms previous work demonstrating the acute-phase antiemetic ability of THC in the Least Shrew (Darmani & Crim, 80 Pharmacol. Biochem. 35-44 (2005); Darmani & Johnson, 488 Eur. J. Pharmacol. 201-2 (2004)) and other species (Kwiatkowski et al., 2004; Van Sickle et al., 2003), and also extends the antiemetic ability of THC to delayed-phase CIV. The CB₁-specific antagonist SR141716A reversed the antiemetic effect of THC, indicating a CB₁-receptor-mediated effect. Furthermore, this effect is mediated at least partially in the DVC, as increased CIV-related Fos-IR in the DVC was attenuated by THC, and restored by prior administration of SR141716A. Both phases of CIV-induced DVC activity, but fewer Fos-IR nuclei were found in shrews vomiting during delayed-phase emesis, suggesting that the anatomical substrate for the pharmacological differences between phases could be differential DVC activation. The most obvious difference was the lack of AP activation in the delayed phase, implying that humoral signaling is unnecessary for induction or blockade of delayed phase vomiting. Interestingly, the number of shrews vomiting in the delayed phase was statistically equivalent to the number vomiting in the acute phase. The protocol of short-term food deprivation followed by mealworm presentation is a reliable method of inducing vomiting in CIS-treated shrews. In addition, the intraperitoneal injection of a bolus of CIS produces a shorter and stronger spike of serum CIS when compared to the normal intravenous, longer term infusion (a 30 minutes or longer infusion period) protocol for cancer patients. It is likely that the combined food deprivation protocol and more intense CIS stimulation improve the reliability of inducing vomiting in the delayed phase, to match that of the acute phase.

Van Sickle et al., 2003, used Fos-IR and correlative pharmacological techniques in ferrets to show that increased Fos-IR in the DVC during the acute phase can be blocked or reduced by THC. The results in the present work in the Least Shrew support these findings: the magnitudes of the increases in Fos-immunopositive nuclei following vehicle injection, or THC injection preceded by SR141716A, were comparable to the ferret, and the reduction in Fos-IR resulting from THC injection was equally similar (id.). When comparing between phases, these results suggest both phases utilize a similar mechanism, with the important exception that the AP is not activated in the delayed phase. The work also brings to light one of the drawbacks of using such a tiny animal as the Least Shrew: the NTS itself is very small, but as in larger animals it appears to subserve several different functions. Although Van Sickle's team was able to identify the medial subnucleus of the NTS as the emesis-related subnucleus according to their Fos data (Tanihata et al., 2003), in the least shrew it was exceedingly difficult or impossible to define the subnuclei of the NTS. Although as in other animals studied, the majority of emesis-induced Fos-IR changes in the Least Shrew NTS would be localized to the medial NTS, further studies with better markers for delineating the NTS subnuclei may confirm this.

These data raise the possibility that peripheral communication via the vagus plays a role in both phases. In both phases of CIV, the DMNX and NTS are stimulated when assessed for Fos-IR. Vagal afferents to the DVC arise from the nodose ganglion, terminate preferentially in the NTS (and to a lesser extent the DMNX), and possess CB₁ immunoreactivity (Koga & Fukada, 14 Neurosci. Res. 166-79 (1992); Onishi et al., 136 Auton Neurosci. 20-30 (2007); Van Sickle et al., 2003). Stimulation of these afferents would produce the pattern of Fos-IR seen following CIV, and presynaptic inhibition of these afferents by THC or other CB₁ agonists would reduce postsynaptic activation and lower Fos-IR. As these vagal neurons also innervate the GIT, stimulation at the enteric level could also induce vagal activity in the DVC. Indeed, THC at large doses can block emesis induced by brain impenetrant, 5-HT₃ receptor-binding emetogens (Darmani & Johnson, 2004), implying that there is a degree of peripheral involvement in vomiting.

Fos-IR measured activation of the DVC during the delayed phase was less than that during the acute phase, although still above baseline in the DMNX and NTS when compared to vehicle-injected controls. The limitations of utilizing Fos-IR for an activity marker have been described thoroughly elsewhere (Dampney et al., 17 Clin. Exp. Hypertens. 197-208 (1995)), but one relevant characteristic of Fos-IR is that it occurs primarily through a recent quantitative increase in neuronal activation. Qualitative changes in activity (e.g., burst vs. static firing) do not consistently induce Fos expression, nor do reductions in basal activity of neurons (e.g., neurons being actively inhibited).

Although CIS stimulates the DVC, and especially the NTS, in the acute phase, no data has been collected studying the electrophysiological activity of DVC neurons in the quiescent or delayed phases. DVC basal activity remaining above pre-injection levels could prevent a subsequent increase large enough to re-induce Fos expression. Alternatively, two subsets of chemically encoded, emesis-activated neurons in the NTS and/or DMNX are present. This is consistent with collected anatomical data. SP and 5-HT have been found colocalized (Thor et al., 2 Synapse 225-31 (1988)), and individually (Boissonade et al., 8 Neurogastroenterol. Motil. 255-72 (1996); Thor et al., 1988), within terminals and neuronal varicosities throughout the DVC. Furthermore, confocal microscopy of multilabeled tissue suggests CB₁ receptors colocalize on putative 5-HT, SP, or 5-HT/SP terminals in the NTS, consistent with published data regarding serotonergic raphe neurons. Thus, CB₁ receptors are in position to modulate both systems either simultaneously or separately, resulting in a pair of overlapping yet neurochemically distinct networks in the DVC.

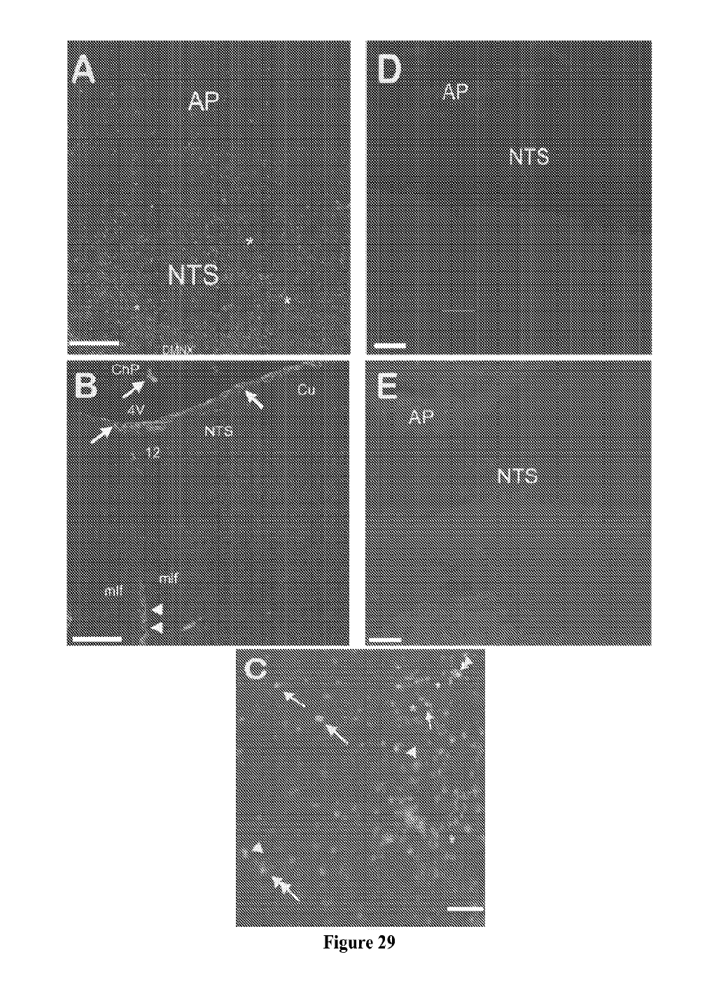

This hypothetical model fits with pharmacological studies demonstrating the antiemetic effects of THC against both serotonergic (Darmani & Johnson, 2004), and tachykininergic (Darmani et al., 2005), emetic stimuli, and with the ineffectiveness of either 5-HT₃ or NK₁ antagonists given individually to completely block CIV (Bountra et al., 1996; Slatkin, 2007). Several studies have found differential neurotransmitter changes (e.g. increased turnover) for both 5-HT and SP in response to CIS in humans, such that serotonergic activity is increased in the acute phase, while tachykininergic activity is increased in both phases, but especially in the delayed phase (Hesketh et al., Eur. J. Cancer, 2003; Higa et al., 12 J. Oncol. Pharm. Pract. 201-09 (2006)). In both cases, patients treated with the combined antagonists responded significantly better than those treated with either antagonist alone without regard to emetic phase. These data suggest that 5-HT dominates activation of the acute phase of emesis, albeit with input from tachykininergic neurons, and that SP dominates activation of the delayed phase. The Fos and IHC data presented herein fit within this hypothetical framework, as CB₁ receptor labeling did colocalize with SP-IR and/or 5-HT-IR in terminal-like structures in the NTS (see FIG. 29C). In control animals, immunolabeling demonstrated that neurons within the NTS activated during emesis (i.e. Fos-IR neurons) were enmeshed within dense CB₁ receptor-immunopositive, putative terminals. Although not conclusive for synaptic contacts, these data are mirrored pharmacologically (Darmani et al., 15TH ANN. SYMP. CANNABINOIDS, 2005; Kwiatkownska et al., 2000; Van Sickle et al., 2003).

Thus, during the acute phase, input from serotonergic and combined (5-HT and SP) projections to the DVC cause a larger increase in numbers of Fos-immunopositive nuclei than that seen during the delayed phase, when mainly tachykininergic inputs to the DVC are being stimulated. The result is that Fos-IR is elevated intermediately between baseline and acute phase CW-related Fos-IR.

The presence of CB₂ receptors in the brain is controversial. Recent reports have purported to find CB₂ receptors in neurons (Onaivi, 54 Neuropsychobio. 231-46 (2007)), although numerous other studies have found putative CB₂ receptors within the vascular endothelium of the brain and other organs (Golech et al., 132 Brain Red. Mol. Brain Res. 87-92 (2004)). In the Least Shrew, CB2-IR did not localize to neuronal structures in the brainstem, but to the surface of the brainstem, the choroid plexus, and to ribbon-like structures within the brainstem which appeared vascular in nature. Data and previous pharmacological data [6,37,41] (Darmani, 24 Neuropsychopharmacology, 198-203 (2005); Simoneau et al., 2001; Van Sickle et al., 2003)), support a non-neuronal localization for CB₂ receptors in the brain and no effect on the emetic reflex. Sources of divergence between studies could include nonspecific binding due to lack of a shrew-specific or ferret-specific antibody. Another possibility is glial or endothelial CB₂ receptors in brain homogenates used to blot for proteins or to recover mRNA, whose presence essentially “contaminates” the samples studied.

One drawback of Fos-based studies is the broad range of Fos-activating stimuli. This can make interpretation difficult in deciphering between Fos-IR related to induction vs. expression (e.g., motor output) of behavior. Although not easily resolved without alternative methodologies (e.g., electrophysiological recording), hypotheses can be drawn based on previous functional data. In the case of the DVC, the NTS is a major integrative site (Davis et al., 1017Brain Res. 208-17 (2004); Onishi et al., 2007), but does not directly generate motor activity. Thus, increased Fos-IR in the NTS is more likely to be related to induction rather than expression of vomiting. The DMNX, however, has both motor output neurons and local circuit cells (Boissonade et al., Neurogastroenterol. Motil., 255-72 (1996); Hornby, 2001; Krowicki & Hornby, 293 293 J. Pharmacol. Exp. Ther. 214-21 (2000)), and could be related to induction and/or expression.

In conclusion, the Least Shrew model provided for herein delivers the first evidence in a freely behaving, emesis-competent species that THC, via CB₁ receptors, effectively inhibits emesis in delayed-phase CIV. Functional anatomy demonstrated that this effect is mediated within the DVC, but without involvement of the AP, and that both emetogenic neurotransmitters, 5-HT and SP, could be inhibited by presynaptic CB₁ receptors to produce THC's antiemetic activity. These data support the limited clinical and anecdotal evidence suggesting that THC or other cannabinomimetics (via stimulation of CB₁ receptors) would be a successful therapeutic modality, or co-therapy, for the prevention of acute and delayed phase chemotherapy-induced vomiting.

The following examples illustrate various methods for compositions in the treatment method of the invention. The examples are intended to illustrate, but in no way limit, the scope of the invention.

### EXAMPLES

### Example 1

### Neurotranmitter Basis of CIV in the Least Shrew

### Animals and Drugs.

Male and female shrews were bred and housed in the Western University animal facilities. Shrews weighing 4 g-6 g (45 days-70 days old) were used throughout the study. The animals were kept on a 14:10-hr light/dark cycle at a humidity-controlled environment in a room temperature of 22±1° C. with ad lib supply of food and water. All animals received care according to the GUIDE FOR THE CARE & USE OF LABORATORY ANIMALS (DHSS Pub., revised, 1985). All of the procedures used in this study were approved by the Institutional Animal Care and Use Committee of the University.

Cisplatin [cis-platinum(II)diamine dichloride [Pt(NH₃)₂Cl₂] was purchased from Sigma/RBI (St. Louis, Mo.) and was dissolved in saline via sonication. It was administered at a volume of 0.1 ml/10 g body weight.

### Behavioral Procedures.

Previously, it was shown that large doses of CIS (10-20 mg/kg, i.p.) produce emesis and abdominal contractions in the Least Shrew within 1 hr-2 hr of administration (Darmani, 1998). In the current behavioral study we investigated: (1) whether CIS can induce both the immediate and delayed phases of emesis and abdominal contractions in a dose-dependent manner in the Least Shrew; (2) the time course of development of these behaviors; and (3) the amount of food ingested during the entire observation time by determining how many mealworms (Tenebrio sp) were eaten per hour by each shrew. Based on preliminary data in the Least Shrew and studies in other animal models of emesis, a few shrews were injected either with a 5 mg/kg or 10 mg/kg (i.p.) dose of CIS and their behaviors were videotaped (Panasonic color CDD camera (WV-CP450) mounted 30 cm above cage) and scored by a trained observer for up to 72 hr post treatment. During the dark night phase a red light bulb was used to illuminate the shrew cage. The attained results indicated that shrews produce both wet vomits (oral ejection of food or liquid following rhythmical undulating contractions of abdomen, thorax and neck muscles with characteristic up and down and forward shaking of the head with mouth openings), dry vomits (all behaviors associated with the process of wet vomiting including mouth openings and head shakings but no actual ejection of food material probably due to an empty stomach), and abdominal contractions (squeezing contractile movements of the hind portion of the Least Shrew involving contractions of abdominal, lateral and back muscles from caudal to cranial direction lasting 2-3 sec without involvement of head movements or mouth openings). In addition, the second phase of emesis tended to decline from 45 hr post CIS injection. Thus, a 47 hour observation period was chosen for the CIS dose-response emesis studies.

To habituate the shrews to the test environment, each animal was randomly selected and transferred to a 14×17×12 cm clean opaque plastic cage and was offered ten mealworms 1 hr prior to experimentation. Then, different groups of shrews were injected intraperitoneally with either vehicle (saline at 0.1 ml/10 g body weight, n=8) or varying doses of CIS (5, 10, and 20; n=7-8 per dose) at 9:00 AM. Immediately following injection, each shrew was returned to its cage and their behaviors were videotaped for the next 47 hr. Animals were allowed ad libitum access to food (mealworms) and water. Food supply was maintained by making sure that two meal worms were always available in the cage throughout the observation time. Water was available throughout the experiment via small plastic container (1 cm in diameter and 0.5 cm in height) glued to the floor of each cage. The mean frequencies (±SEM) of vomitings (wet, dry and total) and abdominal contractions per hour and the number of mealworms eaten per hour, were scored separately from videotape recordings for each individual shrew for the entire observation period by a trained observer blind to the experimental conditions. The 20 mg/kg dose of CIS was toxic to shrews and animals generally expired from 30 hr post-CIS exposure and observation was terminated.

### Neurotransmitter Detection and Analysis Procedures.

The behavioral experiments indicated that a 10 mg/kg dose of CIS produces robust peak frequencies of emesis at 2 hr for the immediate phase and 33 hr post-treatment for the delayed phase without mortality in the 47 hour observation period. These parameters were used in our neurochemical studies. Prior to experimentation, shrews were habituated to the laboratory environment as described for the behavioral studies. A large number of shrews were then injected intraperitoneally (i.p.) with either vehicle (n=6-8 per observation time) or CIS (10 mg/kg, n=6-8 per observation time) for monoamine and SP determination in separate experiments. Subgroups of each treatment were sacrificed by rapid decapitation at either 2 hr or 33 hr post-injection.

### Monoamine Detection Procedures.

Brains were rapidly removed and dissected on an ice-cold petri dish, the frontal cortex and brainstem were immediately frozen on dry-ice and stored at 80° C. until analysis. Likewise, each gut was rapidly removed and washed with ice-cold saline three times prior to sectioning into succeeding 2 cm portions starting from the end of the stomach (duodenal and jejunal sections, respectively), followed by rapid freezing as described above. Each frontal cortex, brainstem and intestinal sample was respectively homogenized (tissue tearer, setting 5 sec for 15 sec) in 1 ml, 2 ml, or 3 ml ice-cold 0.2 M perchloric acid respectively containing 0.3 M ethylenediamine-tetraacetic acid disodium (EDTA). A 0.1 ml portion of each homogenate was removed for protein analysis. The protein concentration was determined by the use of BCA protein assay kit (Pierce; Rockford, Ill.). Then, homogenates were centrifuged at 40,000 g for 30 min at 4° C. to precipitate protein. The supernatant was filtered through a 0.2 μm nylon filter in a microcentrifuge tube at 1,500 g for 10 min at 4° C. A 10 μl aliquot of each filtered supernatant was assayed for the presence of monoamines and their metabolites.

Monoamines and their metabolites were analyzed by high performance liquid chromatography with electrochemical detection (HPLC-ECD) based upon our established method (Darmani et al., 2003). Separation of 5-HT, dopamine and their corresponding metabolites (5-HIAA and HVA) from other electroactive compounds was achieved on a 10 cm×3.2 mm RP-C18 column (ODS 3 um packing; BAS Inc., W. Lafayette, Ind.) and a mobile phase containing 0.15 M chloroacetic acid, 0.12 M sodium hydroxide, 0.18 mM ethylenediamine-tetraacetic acid disodium (EDTA), 1.1 mM sodium octane sulfonic acid and 5% acetonitrile. The monoamines and their metabolites were separated and measured through the cited column via a Shumadzu L-ECD-6A detector, connected to a Shimadzu LC-10 AD pump and a C-R7Ae chromatopac integrator (Kyoto, Japan). The glassy carbon working electrode was set at a potential of 650 mV relative to an Ag/AgCl reference electrode. The detection limit for dopamine and 5-HT were 2 and 5 pg respectively, based on a signal to noise ratio of 3:1. Peak heights of unknowns were compared to the mean peak heights of a 200 pg standard solution (containing 5-HT, dopamine, 5-HIAA and HVA) which were run daily after every fifth tissue supernatant sample. The intestinal HVA concentrations could not be determined due to an unknown interfering peak in the gut tissue eluates.

### Substance P Detection Procedures

Preliminary sample preparation procedures were carried out to optimize detection conditions in accord with our published methods (Darmani et al., 2008). Brain tissue supernatant samples did not show interference to the antibody or the enzyme used in the enzyme immunoassay (EIA) detection kit (Assay Designs, Ann Arbor, Mich.). SP-spiked gut samples showed degradation probably due to presence of excessive endogenous proteases. Inclusion of several cocktails containing protease inhibitors (e.g. pP8340 from Sigma-Aldrich, St. Louis, Mo., or 04693116001 from Roche Applied Science, Indianapolis, Ind.) even at high concentrations [40 mg in 50 μl added in 500 μl of homogenized gut (2 cm suspension)] failed to prevent SP degradation in SP-spiked gut samples. As proteases can be also deactivated by heat, at basic pH and in the presence of EDTA (Darmani et al., 2008), the entire intestine was immediately immersed in a 70° C. preheated for 5 min, 5 ml solution of normal saline containing 10 mm EDTA at pH 8.0 in accordance to published methods (Darmani et al., 2008). Each sample was allowed to cool down naturally to room temperature and then each gut was thrice flushed and rinsed with 5 ml of the cited saline solution. Then, each gut was dissected and the duodenum and jejunum samples were placed separately in test tubes containing 1 ml assay buffer. The dissected frontal cortex and brainstem samples were placed in different test tubes containing 0.5 ml assay buffer. Each sample was homogenized using a tissue tearer (Biospec) set at level 5 for 15 sec. The homogenates were then centrifuged at 17,000×g for 15 min at 4° C. The supernatants were obtained and saved at 80° C. until used for the EIA detection of SP. Samples were analyzed for SP using the “Correlate-EIA” kit from Assay Designs using a microplate reader.

### Statistical Analyses

The frequencies of behavioral data were analyzed by a two-factor repeated measures analysis of variance for CIS dose (0 mg/kg and 10 mg/kg) and time (immediate and delayed phase). When a significant dose and exposure interaction was found, then simple effects at a particular emesis phase was tested via a one-way ANOVA. Neurochemical data were initially analyzed by two-way analysis of variance (ANOVA) with drug dose (0 mg/kg and 10 mg/kg) and exposure time (2 hr and 33 hr) as factors. When dose and exposure factors significantly interacted, simple effects were examined by a one-way ANOVA. For each statistical test, a P value of<0.05 was necessary to achieve statistical significance.

### Example 2

### Substance P and NK, Receptor Antagonists

### Animals and Drugs.

Least Shrews (C. parva) were used as test animals which were bred and maintained in Western University animal facilities. Both male and female shrews (4-5 g, 35-days-old to 60-days old) were used only once in the current study. The feeding and maintenance of shrews are described fully elsewhere. Darmani, 1998; Darmani et al., 1999. All animals received care according to the GUIDE FOR THE CARE & USE OF LABORATORY ANIMALS (DHSS Pub., revised, 1985). All animal protocols were approved by the Western University Animal Care and Use Committee and followed the current guidelines recommended by NIH.

Substance P (SP) and its stable shorter analog, acetyl-[Arg, Sar⁹, Met (O₂)¹¹]-SP (6-11) (ASMSP), were purchased from Sigma/RBI (St. Louis, Mo.).

The following drugs were purchased from Tocris Cookson Inc. (Ellisville, Mo.):

GR73632 [delta Ava [L-Pro⁹, N-MeLeu¹⁰]SP (7-11)];

GR159897 [5-Flouro-3-[2-[4-methoxy -4-[[(R)-phenylsulphinyl]methyl]-1-piperidinyl]ethyl]-1H-indole];

GR64349 [[Lys³, Gly⁸-R-gamma-lactam-leu⁹]Neurokinin-A (3-10)];

L733060 hydrochloride [2S, 3S)-3-[[3,5-bis (Trifluoromethyl)phenyl]methoxy]-2-phenylpiperidine hydrochloride];

SB218795 [2-(Phenyl-4-quinolinyl)carbonyl]amino]-methyl ester benzene acetic acid]; and

[Sar⁹, Met (O₂)”]-Substance P (SarMet-SP).

[Sar⁹, Met (O₂)“]-SP-saporin (SSP-saporin) was purchased from Advanced Targeting Systems (San Diego, Calif.).

CP99,994 [(2S, 3S)-Cis-3 (2-methoxybenzylamino-2-phenylpiperidine) was obtained from Pfizer. Inc. (Groton, Conn.).

A number of drugs (GR159897; CP99,994; L733060 and SB218595) were initially dissolved to twice the stated concentrations in a 1:1:18 solution of ethanol:emulphor:0.9% saline. The drug concentrations were then diluted by the addition of an equal volume of saline. Other drugs were dissolved in H₂O and diluted in saline prior to administration. All drugs were administered at a volume of 0.1 ml/10 g of body weight.

Dose Response Emesis and Scratching Studies with Tachykinin Receptor Agonists and antagonists:

The present protocols were based upon preliminary studies as well as published findings in the Least Shrew. Darmani, 2001a; Darmani et al., 69 Pharmacol. Biochem. Behay. 239-49 (2001b). On the test day, the shrews were allowed to acclimate to the test laboratory for at least one hour prior to experimentation. To habituate the animals to the test environment, each shrew was randomly selected and transferred to a 20 cm×18 cm×21 cm clean clear plastic cage and offered four mealworms (Tenebrio sp.) 30 min prior to experimentation.

The preliminary agonist studies indicated that i.p. administration of both SP and the NK₁ receptor selective agonist GR73632 induce emesis while GR73632 injection also concomitantly produces scratching in the Least Shrew. To evaluate whether i.p. administration of nonselective (SP) or several selective NK₁ receptor agonists (GR73632 (Hagan et al., 19 Neuropeptides, 127-35 (1991); ASMSP (Engberg et al., 73 Biochem. Pharmacol. 259-69 (2007); Sar Met-SP (Wiley & Lappi, 1999); SSP-saporin (Wiley & Lappi, 1999) a selective NK₂ receptor agonist (GR64349 (Hagan et al., 1991)); and a selective NK₃ receptor agonist (Pro⁷-NKB (Salthun-Lassale et al., 68 Mol. Pharmacol. 1214-24 (2005)) can induce emesis or scratching in a dose-dependent manner, different groups of shrews were injected with varying doses of either SP (0, 10, 25, 50, or 100 mg/kg, n=7-11 shrews per group), GR73632 (0, 1, 2.5, or 5 mg/kg, n=8-12 shrews per group), ASMSP (0, 5, 10, and 20 mg/kg, n=6-10 per group), Sar Met-SP (0, 1, 5, and 10 mg/kg, n=4-10 per group), SSP-saporin (0 and 1.2 mg/kg, n=9-10 per group), GR64349 (0, 10 mg/kg, n=8-12 per group), or Pro⁷-NKB (0, 10 mg/kg, n=6-10 per group) Immediately following injection, each shrew was placed in the observation cage and the frequency of vomiting (oral ejections of food or liquid or the act of vomiting which did not result in actual ejection of food or liquid due to an empty stomach; mean±S.E.M.) was recorded for each individual shrew for the next 30 min. These data showed that i.p. administration of 50 mg/kg SP produces a maximal and robust frequency of emesis while a 5 mg/kg dose of GR73632 causes a maximal frequency of emesis and a robust number of scratchings. The latter doses of SP and GR73632 were chosen for subsequent antagonist interaction studies to pharmacologically characterize the role of NK₁ receptors in the mediation of induced emesis by the use of: (1) two selective neurokinin NK₁ receptor antagonists (CP99,994 and L733060) (Watson et al., 115 Br. J. Phramacol. 84-94 (1995); Guiard et al., 89 J. Neurochem. 54-63 (2004)); (2) an NK₂ receptor antagonist (GR159897) (Beresford et al., 272 Eur. J. Pharmacol. 241-48 (1995)); and (3) an NK₃ receptor antagonist (SB218795) (Salthun-Lassalle et al., 1995).

For the above drug interaction studies, different doses (i.p.) of either CP99,994 (0, 5, 10, and 20 mg/kg, n=10-14 shrews per group); L733060 (0, 5, 10, and 20 mg/kg, n=10-14 shrews per group), GR159897 (0 mg/kg and 20 mg/kg, n=8 per group) or SB218795 (0 or 20 mg/kg, n=7-8 per group) were administered to different groups of shrews 30 min prior to injection of an emetic dose (5 mg/kg, i.p.) of the NK₁-receptor agonist GR73632. Emesis and scratchings were recorded for 30 min immediately following GR73632 administration as described above. The antiemetic effect of CP99,994 (0, 5, and 10 mg/kg, n=9-10 animals per group) was also investigated in a similar manner against SP (50 mg/kg, i.p.)-induced emesis.

### Analysis of SP in Serum, Gut and Brain Tissues Following Intraperitoneal Injection of an Emetic Dose of SP:

Different groups of shrews were injected with a 50 mg/kg emetic dose of SP (i.p.) and were sacrificed at 5 min, 15 min, and 30 min (n=5-6 animals per group) post injection. Blood serum as well as two different brain (frontal cortex and brain stem) and gut (duodenum and jejunum) tissue samples were collected for the determination of SP levels relative to a saline-treated shrew control group (n=6). The brain SP concentration was determined in all of the cited exposure times, whereas gut SP tissue levels were analyzed for the 0 min (control), 5 min and 15 min exposure periods Likewise, serum SP concentrations were determined at the 0 min, 5 min and 15 min exposure periods as well as in a 10 min SP-exposed shrew group.

To collect blood, shrews were rapidly euthanized with isoflurane (Vedco, St. Joseph, Mo.) one at a time. Then, each shrew was laid on its back and taped to a metal tray, and cut transversely under the diaphragm and along the sides towards the arms to allow the ribcage to be lifted away. The heart was then held with forceps, and a 0.5 cc syringe with 28 ga hypodermic needle was inserted into the left ventricle. Blood was pulled gently into the syringe directly from the ventricle until the descending aorta appeared clear (approximately 50 μl -100 μl total volume), and rapidly transferred to a non-heparinized tube for centrifugation and removal of red blood cells. Brain and gut tissue were rapidly dissected following blood collection and decapitation. Preliminary sample preparation procedures were carried out to optimize detection conditions. Brain tissue supernatant and blood serum samples did not show interference to the antibody or the enzyme used in the enzyme immunoassay (EIA) detection kit (Assay Designs, Ann Arbor, Mich.). SP-spiked gut samples showed degradation, however, probably due to presence of excessive endogenous proteases. Inclusion of several cocktails containing protease inhibitors (e.g., pP8340 from Sigma-Aldrich, St. Louis, Mo., or Cat. #04693116001 from Roche Applied Sci., Indianapolis, Ind.), even at high concentrations (40 mg in 50 μl added in 500 μl of homogenized gut (3 cm suspension), failed to prevent SP degradation in SP-spiked gut samples.

Because proteases can be deactivated by heat (El-Salhy, 107 Upsala J. Med. Sci. 101-10 (2002)), at basic pH (Crawford & Kalmakoff, 24 J. Virol. 412-15 (1977)), and in the presence of EDTA (Wei & Bobek, 49 Antimicrob. Agents Chemother. 2336-42 (2005)), the entire intestine was immediately immersed in a 70° C. preheated, 5 ml solution of normal saline containing 10 mm EDTA at pH8.0. After a 5 min exposure, each gut was thrice flushed and rinsed with 5 ml of the saline solution and then allowed to cool down naturally to room temperature. Then, each gut was dissected and the duodenum and jejunum samples were placed separately in test tubes containing 1 ml assay buffer. The dissected frontal cortex and brain stem samples were placed in different test tubes containing 0.5 ml assay buffer. Each sample was homogenized using a tissue tearor (Biospec) set at level 5 for 15sec. The homogenates were then centrifuged at 17,000×g for 15 min at 4° C. The supernatants were obtained and saved at −80° C. until used for the EIA detection of SP. Samples were analyzed for SP using the “Correlate-EIA” kit from Assay Designs using a microplate reader.

### Immunohistochemistry of Fos and Tachykininergic Systems:

Shrews were given four mealworms each and GR73632 (2.5 mg/kg, i.p., or vehicle (saline)) injection. For emesis-related Fos visualization, a shrew was transcardially perfused 60 min to 75 min after vomiting occurred, typically 20 min to 35 min post-injection. Animals that did not vomit were perfused 90 min to 100 min post-injection.

After the appropriate time period as described above, shrews were anesthetized with a lethal dose of pentobarbital (10 mg/kg) and perfused transcardially via 25 ga blunted needle connected to a peristaltic pump. The shrew was perfused first with ice cold heparinized saline (0.9% NaC1, 60 sec-90 sec), followed by ice cold 4% paraformaldehyde/5% picric acid in pH7.4, 0.1 M (10 min). Brains were removed and stored in 30% sucrose in 0.1 M phosphate buffer (PB) overnight, then embedded in blocks of 12% gelatin in 30% sucrose/PB. The blocks were postfixed for 3 hr in 2% paraformaldehyde/PB, then rinsed and immersed in 30% sucrose/PB until they sank (usually 1 hr to 2 hr). The brain block was cut on a freezing benchtop microtome (Leica) at 30 μm into five series, and stored in PB with 0.03% sodium azide.

Immunohistochemistry was performed by blocking free-floating sections with 10% normal horse serum (NHS) and 3% hydrogen peroxide in PB with 0.3% Triton X-100 (TX) for 30 min After rinsing three times in PB, tissue was put in either: (1) rat anti-Substance P monoclonal antibody (Chemicon, 1:400); (2) sheep anti-Fos polyclonal (Chemicon, 1:600); or (3) rabbit anti-NK₁ receptor polyclonal (Santa Cruz, 1:500), with 5% NHS and 0.3% TX, and incubated overnight at room temperature with gentle shaking. After rinsing three times in PB, the tissue was placed in biotinylated donkey anti-rat, anti-sheep, or anti-rabbit (respectively) IgG secondary antibody (Jackson Immuno, dilution 1:600). The secondary antibody was diluted in the same diluent described for the primary antibody, and the tissue incubated at room temperature with shaking for 75 min. After rinsing three times in PB, tissue was incubated for 60 min in horse radish peroxidase-conjugated avidin-biotin complex (Vector Labs Vectastain kit, diluted 1:2) in PB. Tissue was then rinsed twice in PB, then once in imidazole-acetate buffer (0.1 M, pH7.4), then reacted for 6 min in 0.0006% hydrogen peroxide with 2% nickel ammonium sulfate-enhanced diaminobenzidine (DAB, 0.05% in 0.1 M imidazole-acetate buffer).

After reacting, the tissue was rinsed thoroughly in PB and mounted onto gel-subbed slides out of PB. After air-drying, slides were dehydrated through a series of ascending ethanols (50%-75%-90%-100%), then cleared in xylene (Fisher). Cleared slides were coverslipped with DEPEX (Electron Microscopy Sciences).

Photomicrographs of regions of interest were taken at 1600×1200 pixel digital resolution with a SPOT digital camera (Diagnostic Instruments) attached to a Pentium 4 PC running version 4.0 of the SPOT software. The camera was mounted to a Nikon Eclipse E600 microscope. A calibrated scale bar was added and the photos exported to Adobe Photoshop 7, where the Photoshop brush tool was used to mark Fos+nuclei as they were counted. Relevant structures were identified using an atlas produced in lab (Ray & Darmani, 2007). Cell counts for each structure were averaged on a per-section basis and collated in a spreadsheet for statistical analysis.

SSP-Saporin Studies:

To better localize the induction of emesis by GR73632, dose-response curves were generated for shrews pretreated either with the NK₁ receptor immunotoxin SSP-saporin or saline and challenged four days later with varying doses of GR73632 (0, 1, 2.5, or 5 mg/kg, i.p., n=8-10 per group). Briefly, thirty shrews were injected i.p. with 1.2 mg/kg SSP-saporin in sterile saline, and thirty-five shrews were injected with sterile saline alone. In addition, two more shrews were injected with 1.2 mg/kg unconjugated saporin (saporin; ATS) and two more with 1.2 mg/kg of saporin conjugated to a nonspecific peptide sequence (Blank-saporin; ATS). Shrews were returned to the animal facility for three days and supplied with unrestricted food and water. Saporin-injected and Blank-saporin-injected shrews were perfused four days post-injection, and brain and gut harvested as described for immunohistochemical staining. The SSP-saporin and saline-injected shrews were divided into different groups on day 4 post-injection, and each group was injected i.p. with varying doses varying doses of 0, 1, 2.5, or 5 mg/kg GR73632 dissolved in saline. Each shrew was monitored for 30 min post-injection and vomiting and scratching behaviors quantified. Three shrews from each group were then perfused 90 min to 120 min post-injection as described previously for Fos immunohistochemistry, and brain and gut harvested. Immunohisto-chemistry (IHC) was performed for SP, NK₁ receptor, and Fos as described, to qualitatively examine the extent of immunotoxic lesion in the brain and/or enteric nervous system. IHC for SP and NK₁ receptor was done on the saporin- and Blank-saporin-injected shrews to verify the specificity of SSP-saporin and lack of toxicity of unconjugated saporin.

### Statistical Analysis:

The data on the frequency of emesis and scratchings were analyzed by the Kruskal-Wallis (KW) nonparametric one-way analysis of variance (ANOVA) and post hoc analysis by Dunn's multiple comparisons test or Mann-Whitney test. A P value of <0.05 was necessary to attain statistical significance. The incidence of emesis (number of shrews vomiting) was analyzed by the Fisher's exact test to determine whether there were differences between groups. When appropriate, pairwise comparisons were also made by this method. Two-way analysis of variance using the Kruskal-Wallis or Fisher's exact tests were initially utilized to respectively analyze the dose-response results of GR73632 on emesis and scratching behaviors, and percentage of shrews vomiting in saporin- and corresponding saline-pretreated control shrews. The data did not converge, however, and consequently the described one-way ANOVA statistical methods were appropriately used to analyze the data. For some emesis data, the two-tailed Mann-Whitney test was used. The SP tissue concentration data were analyzed by a one-way ANOVA followed by Dunnett's t test as post hoc analysis. Fos-IR was quantified by counting immunopositive nuclei within each hemisection containing the region of interest (ROI). The number of nuclei was averaged across sections with the ROI for each animal, and the group mean and standard error obtained. Variance was checked by one way ANOVA to ensure the groups were not statistically different, and a 2-tailed student's t-test was used for significant differences between group means.

Dose-Response Emesis and Scratching Studies with Tachykinin Receptor Agonists and Antagonists:

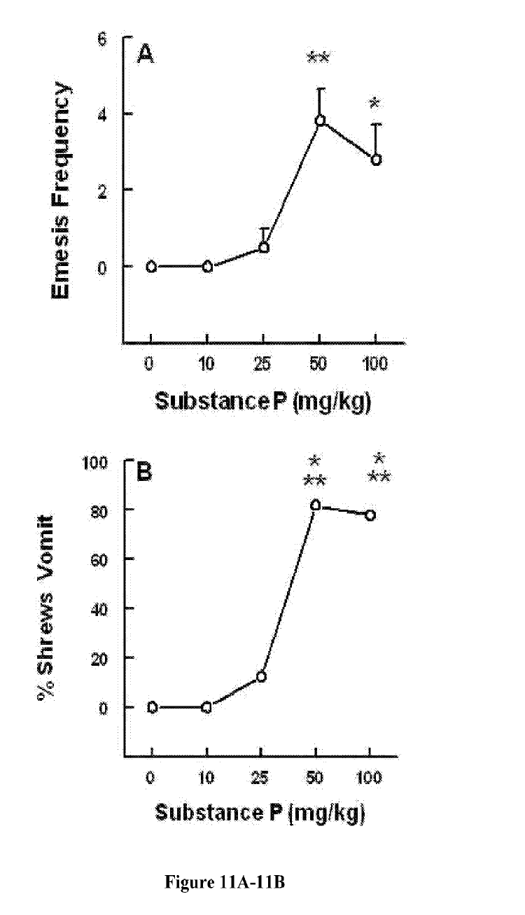

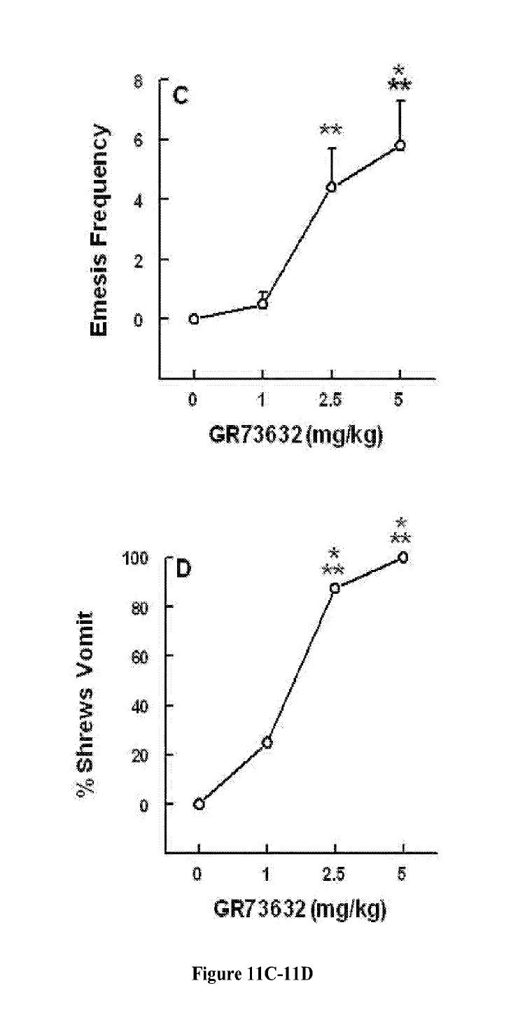

Intraperitoneal administration of SP (0, 10, 25, 50, and 100 mg/kg) increased the frequency of vomiting (KW (4, 40)=25.7, p<0.0001) (FIG. 11A). Dunn's multiple comparisons posthoc test showed that relative to the vehicle-treated control group, significant (382% (p<0. 01) and 322% (p<0.05)) increases in the frequency of vomiting occurred in groups injected with the 50 mg/kg and 100 mg/kg doses of SP. The 10 and 25 mg/kg doses of SP were inactive. The onset of first emesis was rapid within 1-2 min of SP injection except one animal that vomited at 24 min. The remaining episodes of vomiting were scattered at various times within 25 min of injection. Fisher's exact test showed that the percentage of shrews vomiting in response to SP administration increased in a dose-dependent manner (χ²(4, 40)=27.7, p<0.0001) (FIG. 11B). Significant increases (82% and 78%, respectively) in the number of shrews vomiting were seen at 50 mg/kg (p<0.001) and 100 mg/kg (p<0.001) doses of SP. Although in initial dose-response studies not all shrews vomited in response to either 50 or 100 mg/kg doses of SP, in subsequent drug interaction studies, all vehicle-pretreated animals vomited in response to 50 mg/kg SP injection. At the doses tested, SP caused no other overt behavioral effect (e.g. scratching).

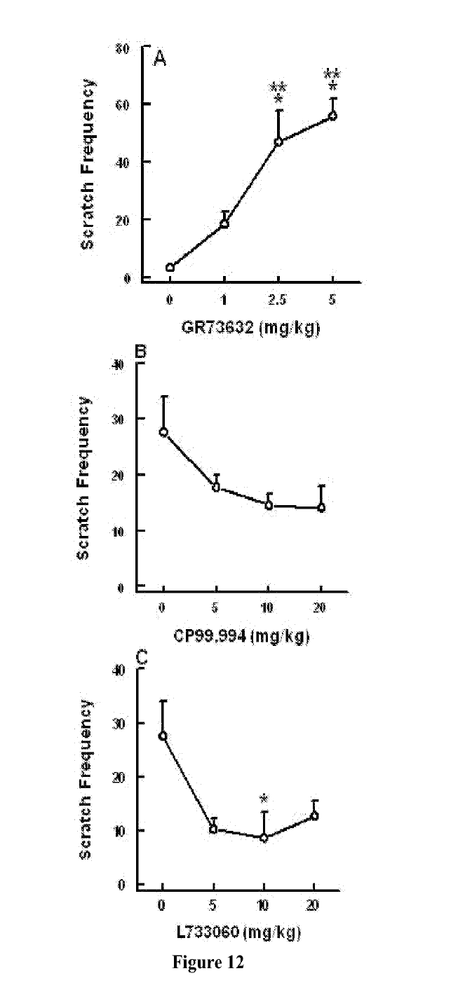

The brain-penetrating and selective NK₁ receptor agonist GR73632 (0, 1, 2.5, and 5 mg/kg) increased the frequency of vomiting in a dose-dependent manner (KW (3, 32)=24.9, P<0.0002) (FIG. 11C). Significant increases in emesis frequency occurred at 2.5 mg/kg (438%, p<0.01) and 5 mg/kg (575%, p<0.001) doses. The percentage of shrews vomiting also increased in a dose-dependent fashion (χ² (3, 32)=26.5, P<0.0001) and significant enhancements (87.5% and 100%) in the number of shrews vomiting were observed at 2.5 (P<0.001) and 5 mg/kg (p<0.001) doses (FIG. 11D). The onset of first emesis was rapid and generally occurred within 3-4 min of GR73632 administration and the remaining episodes occurred in the next 15 min Although SP failed to cause scratchings, intraperitoneal injection of GR73632 also caused dose-dependent increases in the frequency of scratching behavior (KW (3, 30)=24, 36, P<0.0001) (FIG. 12A). Significant enhancements were seen at 2.5 mg/kg (P<0.001) and 5 mg/kg doses (p<0.001) (FIG. 12A). The CNS non-penetrating NK₁ receptor agonists produced less robust emetic and scratching behaviors which were not significantly different from their corresponding vehicle-treated controls. Indeed, ASMSP caused emesis respectively in 37%, 50% and 50% of tested shrews at its 5 mg/kg, 10 mg/kg and 20 mg/kg doses. It also caused 10 to 15 scratchings but the effect was not dose-dependent. The third selective NK₁ receptor agonist, Sar Met-SP, at doses of 1, 5, and 10 mg/kg caused emesis in 20%, 33% and 0% of shrews, respectively. Likewise, it induced 4 to 10 scratchings which were nondose-dependent. The saporin analog of the latter agent was tested at 1.2 mg/kg dose and caused emesis in 1-out-of-9 tested shrews.

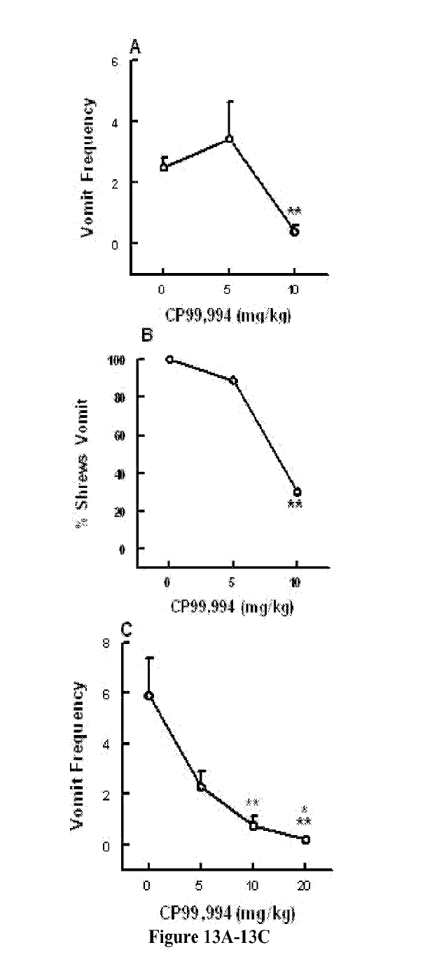

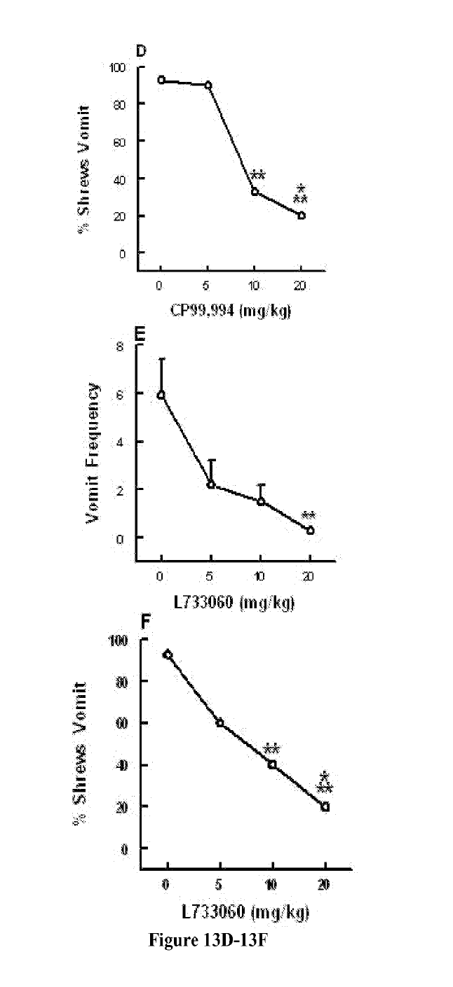

In FIG. 13, panels A and B reflect the antivomiting activity of the NK₁ receptor antagonist CP99,994 (0, 5, and 10 mg/kg, i.p.) against a 50 mg/kg (i.p.) emetic dose of SP. CP99,994 significantly attenuated (84%, P<0.01) the frequency of induced emesis (KW (2, 26)=13.6, p<0.001) as well as protecting (70%, P<0.003) shrews from emesis (χ² (2, 26)=14, P<0.001) at a dose of 10 mg/kg (FIGS. 13A and 13B). CP99,994 (0, 5, 10, and 20 mg/kg) also dose-dependently reduced both the frequency [(61%, 87%, and 97%, respectively) (KW (3, 42)=21.2, P<0.0001)] and the percentage of (2.9%, 60%, and 73%, respectively) shrews vomiting (χ² (3, 42)=26.41, P<0.0001) in response to a 5 mg/kg dose GR73632 (FIGS. 13C and 13D). Indeed, significant reductions in both GR73632-induced emetic parameters were seen at its 10 mg/kg (P<0.01) and 20 mg/kg (P<0.001) doses. Although CP99,994 (0, 5, 10, and 20 mg/kg) also caused up to 49% reduction in the frequency of scratching, the reduction failed to attain significance (FIG. 12B) Likewise, the second tested NK₁ receptor antagonist , L733060 (0, 5, 10, and 20 mg/kg) attenuated both the frequency (62% (P>0.05), 74% (P>0.05), and 95% (P<0.01), respectively, (KW (3, 40)=14.4, P<0.002) and the percentage of shrew vomiting (33% (P>0.05), 53% (P<0.01), and 73% (P<0.001), respectively) (χ²(3, 40)=14.1, P<0.02) in response to GR73632 (FIGS. 13E and 13F). Both CP99,994 and L733060 only delayed the onset of first emesis induced by GR73632 at their highest tested effective antiemetic doses. L733060 also reduced (63%, 69%, and 54%, respectively) the GR73632-induced scratchings (KW (3, 40)=3, P<0.043) and a significant reduction was seen at its 10 mg/kg dose (P<0.05) (FIG. 12C).

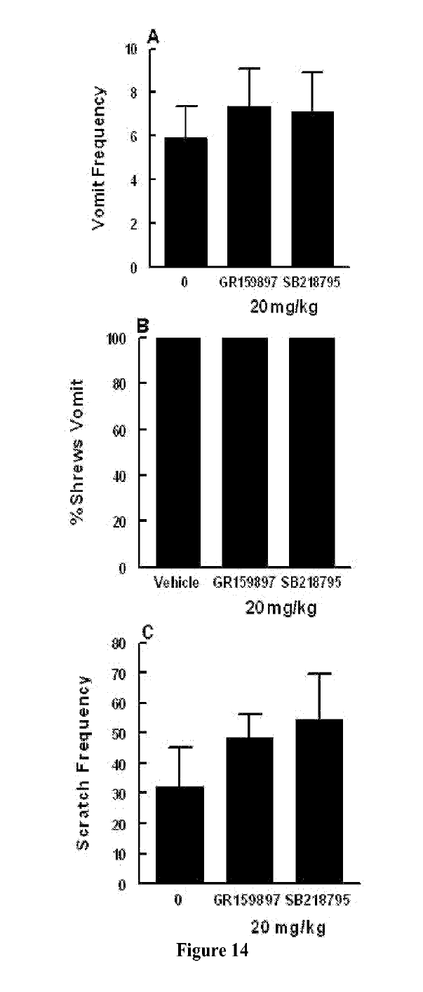

Although pretreatment with either the NK₂ receptor antagonist (GR159897; 20 mg/kg, i.p.) or the NK₃ receptor antagonist (SB218795; 20 mg/kg, i.p.) tended to increase the frequency of both emesis (FIG. 14A) and scratching (FIG. 14C) induced by a 5 mg/kg dose of the NK₁ receptor agonist GR73632, the enhancements did not attain significance. These antagonists also failed to affect the percentage of shrews vomiting in response to GR73632 (FIG. 14B). Moreover, these antagonists did not produce any behavioral effect by themselves at a 20 mg/kg dose. In addition, neither the selective NK₂ receptor agonist (GR64349) nor NK₃ receptor agonist (Pro7-NKB) produced emesis or significant scratchings at doses up to 10 mg/kg.

### Analysis of SP in Shrew Serum, Gut and Brain:

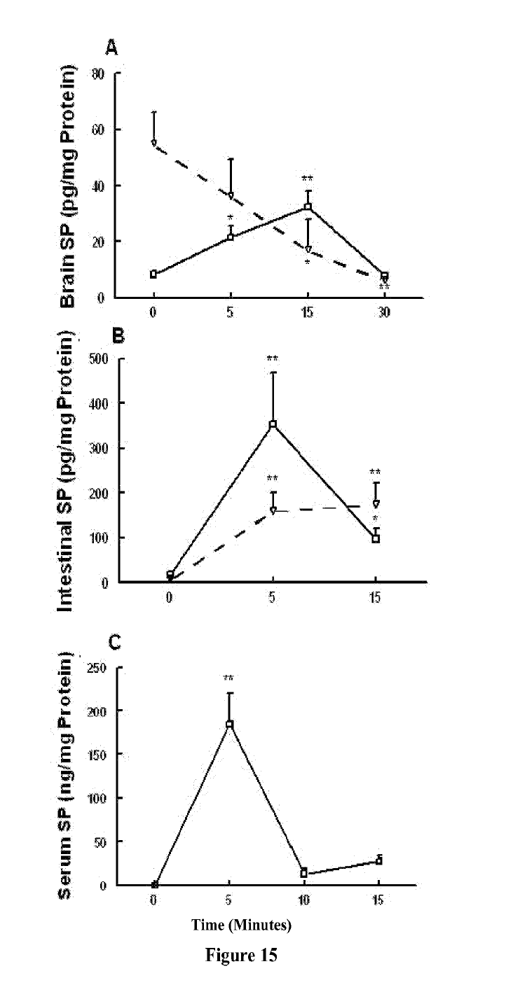

Intraperitoneal administration of a 50 mg/kg dose of exogenous SP initially significantly increased the basal level of brain stem SP in a time dependent manner (F (3,21)=13.8 P<0.0001) (FIG. 15A). Indeed, significant increases occurred at 5 min (156%, p<0.05) and 15 min (287%, P<0.01) post-injection which returned to basal tissue level by 30 min. On the other hand, the basal frontal cortex SP concentration concomitantly decreased [(34, P>0.05), 68% (P<0.05) and 89% (P<0.01), respectively] in a time-dependent fashion (F (3,21)=5.3, P<0.01) FIG. 15A). Both duodenal (1883% increase over basal levels (P<0.01)) and jejunal (2133% increase over basal level, (P<0.01)) tissue levels significantly increased within 5 min of exogenous SP injection which then either remained unchanged or decreased by 15 min of post-administration, but still were significantly (P<0.01 and P<0.05, respectively) higher than their basal values by several fold ((F (2,17)=8.3, P<0.003) and (F (2, 17)=8.9, P<0.002), respectively (FIG. 5B)). Likewise, the basal SP blood serum level dramatically increased by 3488-fold at 5 min post-injection and rapidly declined by 10 min (F (3,15)=29.7, P<0.0001) (FIG. 15C).

### Fos-IR Studies:

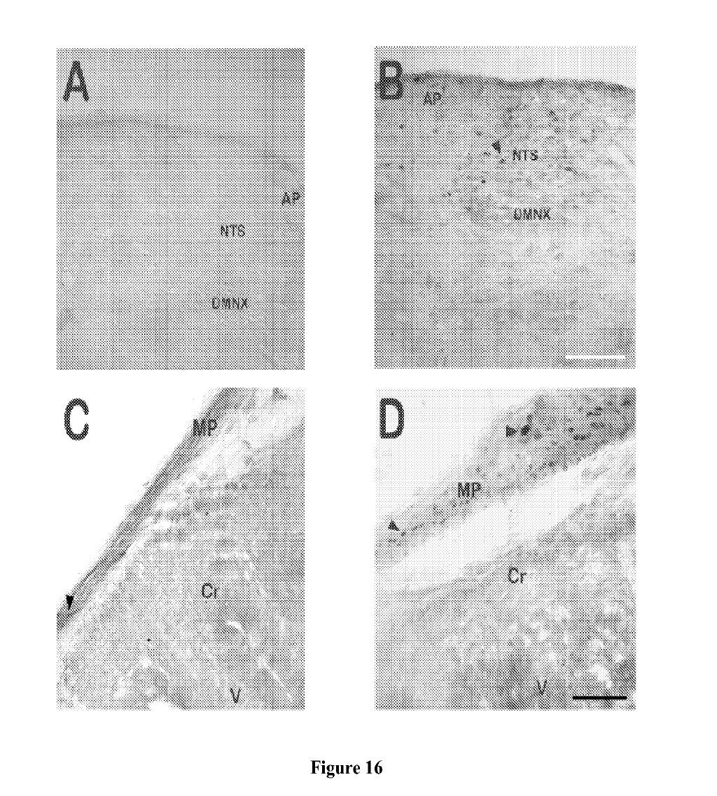

Intraperitoneal injections of the NK₁ receptor agonist GR73632 (2.5 mg/kg) induced vomiting in five of six shrews and retching in the sixth shrew, whereas saline-injected controls (N=6) did not vomit or retch at all. Photomicrographs of Fos immunohistochemistry (Fos-IR) in GR73632 and saline-injected shrews are shown in FIG. 16. The comparison in Table 1 enumerates the mean number of Fos-IR nuclei (mean±SEM) per hemisection in GR73632-injected or saline-injected shrews, and of Fos-IR nuclei per 1 cm sliced length of enteric nervous system (i.e., small intestine):

TABLE 1
Fos-IR nuclei per coronal hemisection after injection of
GR73632 or saline.

| Region of Interest | Saline | GR73632 | Significance level |
|---|---|---|---|
| PFC | 74.1 ± 13.7 | 58.4 ± 14.2 | 0.22 |
| NTS | 2.7 ± 1.0 | 8.2 ± 1.1 | 0.003 |
| AP | 1.0 ± 0.8 | 2.4 ± 1.2 | 0.178 |
| DMNX | 1.2 ± 0.6 | 3.3 ± 1.0 | 0.037 |

The data in Table 1 are presented as mean±standard error for each group and each region of interest. Significant differences between GR73632 and saline injected groups (N=6 per group) were found in the NTS and the DMNX. AP, area postrema; DMNX, dorsal motor nucleus of the vagus nerve; NTS, nucleus of the solitary tract; PFC, prefrontal cortex.

As the data in Table 1 show, significant differences between GR73632 and saline injected control group were found in the NTS, the DMNX and the gastrointestinal enteric nervous system. No significant effect was observed in the AP between the control and treated shrews.

Histological Analysis of NK₁ Receptor Immunotoxic Lesions with SSP-Saporin:

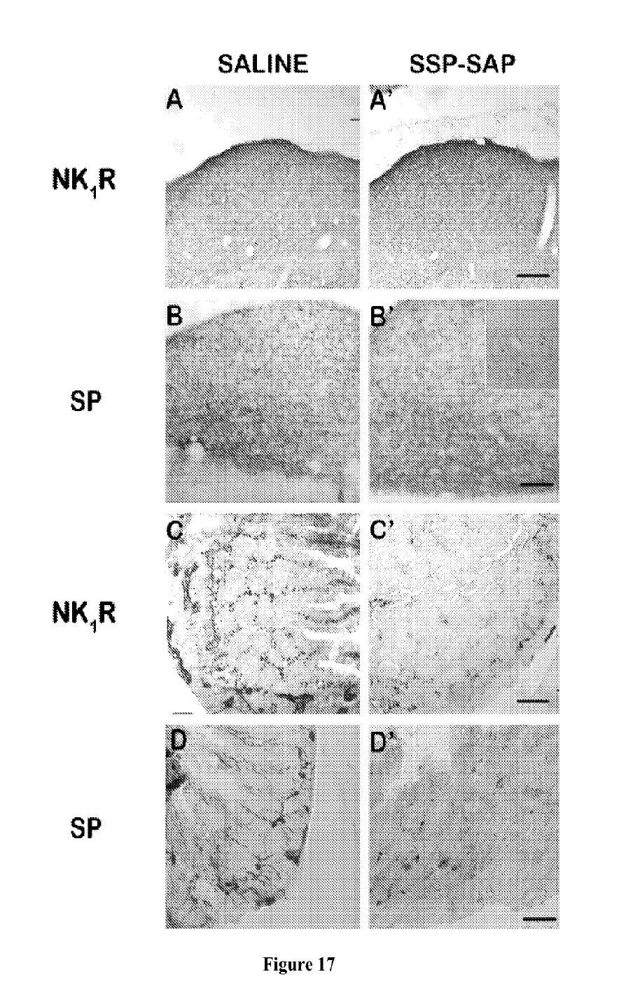

Results of immunolabeling following saporin-based immunolesion are presented in FIG. 17. There was a clear loss of NK₁ receptor-IR in the gut (FIG. 17C), but not the brain (FIG. 17A), of the SSP-saporin injected shrews vs. corresponding saline-treated controls (FIGS. 17C, 17A). In addition, relative to saline-treated controls (FIG. 17D), the innervation of the intestine by SP (FIG. 17D) was greatly reduced—although somata in the myenteric plexus were generally intact, fibers and terminal-like structures normally penetrating the villi and crypts of the intestinal wall were only sparsely present, or completely absent. SP-IR in the brain appeared similar to that of controls (FIG. 17B/B). Fos-positive nuclei following GR73632 administration were found in saline-preinjected controls in the myenteric plexus and the medullary DVC, but only in the DVC in SSP-saporin-preinjected shrews (FIG. 17B inset). NK₁ receptor-IR and SP-IR in shrews injected with saporin or blank-saporin were not different from that found in control shrews.

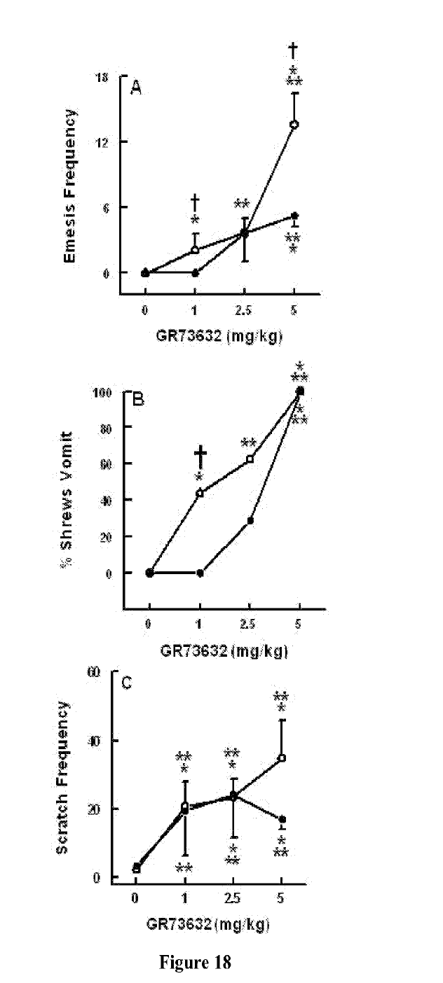

Emesis and scratching studies following NK₁ receptor immunotoxic lesions with SSP-saporin: Challenge administration of different doses of GR73632 (0, 1, 2.5, and 5 mg/kg) in different groups of NK₁ receptor-intact control shrews pretreated with a single injection of saline four days prior to the challenge test procedure caused dose-dependent increases in the frequency of emesis (KW (3, 31)=20.4, P<0.00014) and scratchings (KW (3, 31)=18.2, P<0.0004) as well as the percentage of shrews vomiting (0%, 44%, 62.5% and 100%) (χ² (3, 31)=18.5, P<0.0001) (FIGS. 18A, 18B, 18C). Post-hoc analysis indicated that the emesis frequency, the percentage of shrews vomiting and the number of scratches were respectively increased at 1 mg/kg (P<0.02, P<0.05, P<0.001), 2.5 mg/kg (P<0.006, P<0.013, P<0.001), and 5 mg/kg doses (P<0.001 for all cases). GR73632 challenge in saporin-pretreated NK₁ receptor-ablated shrews also significantly increased the frequency of emesis (KW (3, 26)=19.4, P<0.0001), the percentage of shrews vomiting (0%, 0%, 28% and 100%, respectively) (χ² (3, 26)=22.7, P<0.0001), and the number of scratchings (KW (3, 26)=13.2, P<0.004) (FIGS. 18A, 18B, 18C). Post-hoc analysis indicated, however, that while the frequency of emesis and the percentage of shrews vomiting increased significantly only at 5 mg/kg (P<0.001 in both cases), the number of scratches increased in a significant manner at all tested doses of GR73632 (P<0.02, P<0.001, and P<0.001, respectively). In addition, significant differences were observed among the NK₁ intact control and NK₁-ablated shrews for some doses of GR73632 in emesis parameters but not in the scratching behavior. Indeed, the 1 mg/kg GR73632 dose caused about two vomits in 44% of normal shrews, but was unable to induce emesis in any of the NK₁ ablated shrews (p<0.041 and P<0.04, respectively) (FIGS. 18A and 18B). Although relative to normal shrews a smaller proportion of NK₁-ablated shrews vomited in response to 2.5 mg/kg GR73632, the difference did not attain significance and both groups produced nearly identical mean frequencies of emesis. On the other hand, the 5 mg/kg dose caused emesis in all of the tested animals, however, the NK₁ ablated shrews exhibited significantly less bouts of vomiting (P<0.046). Overall, the percent shrew vomit dose-response curve in NK₁ ablated shrews seems to be shifted to the right of its corresponding control curve in normal shrews (FIG. 18B).

The most dramatic effect of NK₁ receptor ablation on GR73632-induced emesis was qualitative. Indeed, in the NK₁ receptor-intact animals, the duration of emesis with corresponding rhythmic abdominomuscular retching movements in a cephalad direction and accompanying opening of the mouth to expel gastrointestinal contents was approximately 2 sec to 4 sec. On the other hand, in NK₁ receptor-ablated shrews, the process of expulsion of food/liquid was much more prolonged lasting 15 sec to 30 sec. Thus, the discussed retching movements with corresponding ondulatory mouth openings were continuously present until emesis occurred but these animals seemed unable to initiate a significant retroperistaltic gastrointestinal movement to expel the vomit.

### Example 3

### Antiemetic Effects of the CysLT₁ Receptor Selective Antagonist Pranlucast Against Leukotriene LTC₄-Induced Emesis in the Least Shrew

Least Shrews (C. parva) were used as test animals which were bred and maintained in Western University animal facilities. Both male and female shrews (4-5 g, 35-60 days old) were used only once in the current study. The feeding and maintenance of shrews are described fully elsewhere. Darmani, 105 J. Neurol. Transm 1143-54 (1998); Darmani et al., 106 J. Neulol. Transm. 1045-61 (1999); Darmani, 24 Neuropsychopharmacol. 198-203 (2001a)). All animals received care according to the GUIDE FOR THE CARE & USE OF LABORATORY ANIMALS (DHSS Pub., revised, 1985). All animal protocols were approved by the Western University Animal Care and Use Committee and followed the current guidelines recommended by the National Institutes of Health.

Leukotrienes LC₄ and LD₄ and pranlucast (Pranlukast, ONO-1078) were purchased from Cayman Chemicals (Ann Arbor, Mich.). Bay u9773, a non-selective antagonist of the cysteinyl LT receptors, was obtained from Sigma (St. Louis, Mo.).

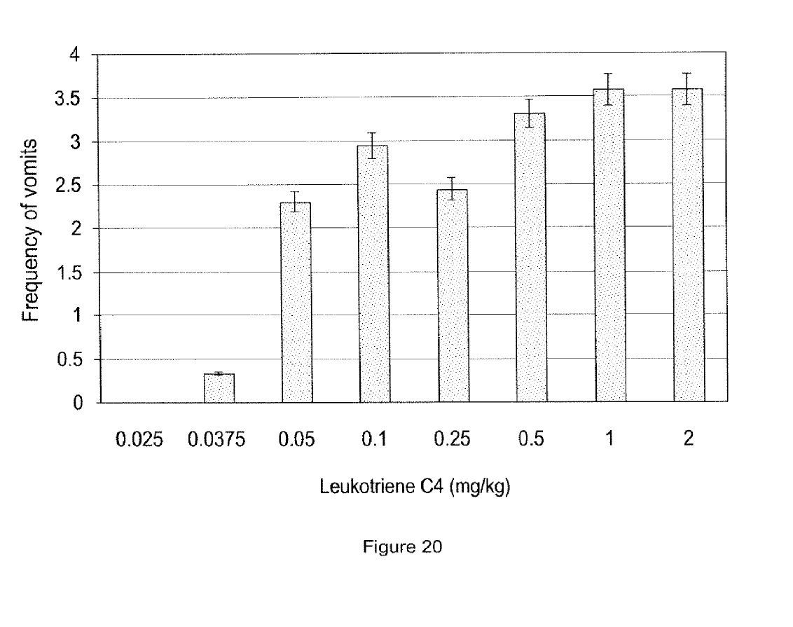

Varying doses of leukotriene LTC₄ were administered by intraperitoneal injection, and dose-response emetic effects recorded for 30 min post-injection observation period. The graph in FIG. 20 depicts enhancements in the frequency (mean±S.E.M.) of emesis.

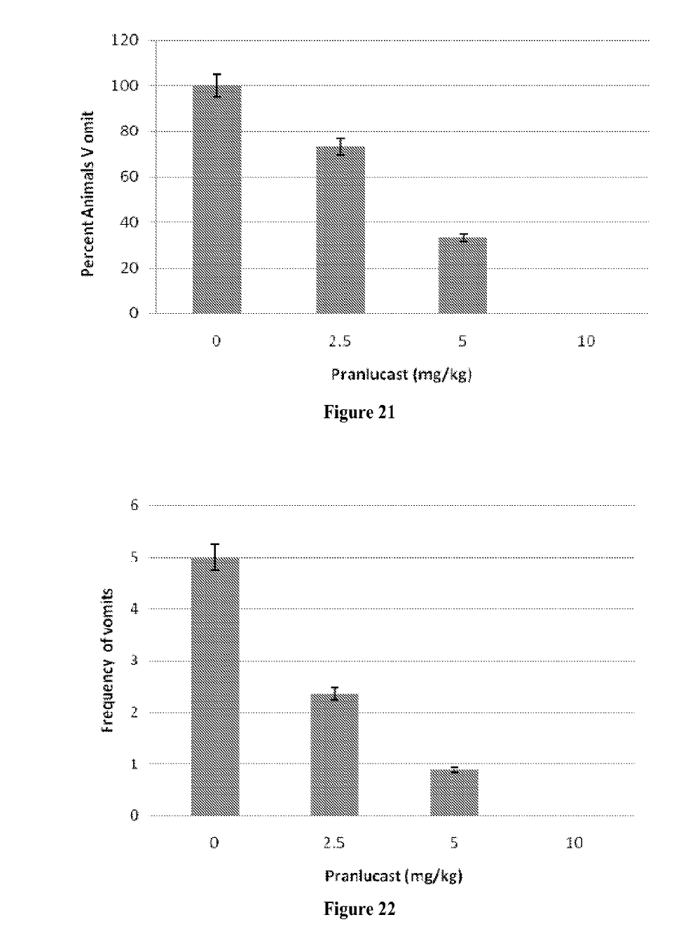

Different groups of shrews received i.p. vehicle (0 mg/kg), or varying doses of the cysteinyl leukotriene antagonist pranlucast (2.5, 5, or 10 mg/kg) 30 min prior to an emetic dose of leukotriene LTC₄ (1 mg/kg). Emetic parameters were recorded for 30min post emetic injection. Attenuations in the frequency (mean±S.E.M.) of emesis are shown in FIG. 21. Enhancements in the frequency (mean±S.E.M.) of emesis are shown in FIG. 22.

### Example 4

### Effect of Δ⁹-THC Alone or in Combination, on the Frequency of CIS-Induced Emesis

### Animals and Drugs.

Both male and female least shrews (C. parva, 4-6 g, 35-60 days old) were used in the study. The feeding and maintenance of shrews have been described previously (Darmani, 1998; Darmani et al., 1999). Animal care was provided and monitored according to the GUIDE FOR THE CARE AND USE OF LABORATORY ANIMALS, DHSS Publication, revised (1985), and all procedures were approved by the Institutional Animal Care and Use Committee of the Western University of Health Sciences. Tropisetron was bought from Research Biochemicals Inc., Natick, Mass., and all other drugs used were purchased from Sigma/RBI (St. Louis, Mo.). Δ⁹-THC, provided through NIDA (Rockville, Md.), and dexamethasone (DEX), were initially dissolved to twice the stated concentrations in a 1:1:18 solution of ethanol/Emulphor™/0.9% saline, and then further diluted by the addition of an equal volume of saline. cis-diammineplatinum(II)dichloride (CIS) and tropisetron were dissolved in distilled water.

### Generation of Dose-Response Curves

The present protocols were based upon our published findings that tropisetron dose-dependently attenuated CIS-induced vomiting (Darmani, 1998), and upon the basic and clinical studies examining combinations of Δ⁹-THC, prochlorperizine, and dexamethasone (Garb, 1981; Hesketh et al., 2006; Kwiatkowska et al., 2004; Lane et al., 1990). Briefly, all animals were randomly divided into two groups. On the test day, shrews were habituated to the laboratory environment for at least 1 hr prior to experimentation. Each animal was transferred to a 20×18×21 cm clean, clear plastic cage and offered four mealworms (Tenebrio sp.) 30 min prior to CIS injection (20 mg/kg, intraperitoneal). In the first group, 10 min prior to CIS administration, shrews were injected with a dose of either: (a) tropisetron alone (0, 0.025, 0.1, 0.25, 1, or 5 mg/kg, i.p., n=8-12), or (b) tropisetron in combination with a dose of Δ⁹-THC (0, 0.1, 0.25, 0.5, 1, or 5 mg/kg, i.p.). In the second group of shrews,10 min prior to CIS administration the animals were injected i.p. with either (a) varying doses of DEX alone (0, 0.25, 1, 5, 10, or 20 mg/kg, n=11-14), or (b) varying doses of DEX in combination with different doses of Δ⁹-THC (0, 0.1, 0.25, 0.5, 1, 2.5, 5, or 10 mg/kg, i.p.). All drugs were administered at a volume of 0.1 ml/10 g body weight. Immediately following the second injection, each shrew was placed in the observation cage, and both the number of animals vomiting per group and the frequency of vomiting (oral ejections of food or liquid) were recorded for the next 60 min

### Statistical Analysis.

Poisson regression and logistic regression analyses were employed for two-way analysis of vomiting frequency and protection from emesis, respectively. Because the results did not converge, a one-factor analysis for various doses of one drug in the presence of a given dose of the second drug was employed. Data regarding the frequency of emesis were analyzed by the Kruskal-Wallis nonparametric one-way analysis of variance (ANOVA), and post hoc analysis by Dunn's multiple comparisons test. The incidence of emesis (number of animals vomiting) was analyzed by Fisher's exact test to determine whether there were differences between groups, and when appropriate, pairwise comparisons were also made. A p value of<0.05 was necessary to achieve statistical significance.

### Effect of Tropisetron and Δ⁹-THC Alone or in Combination on the Frequency of CIS-Induced Emesis.

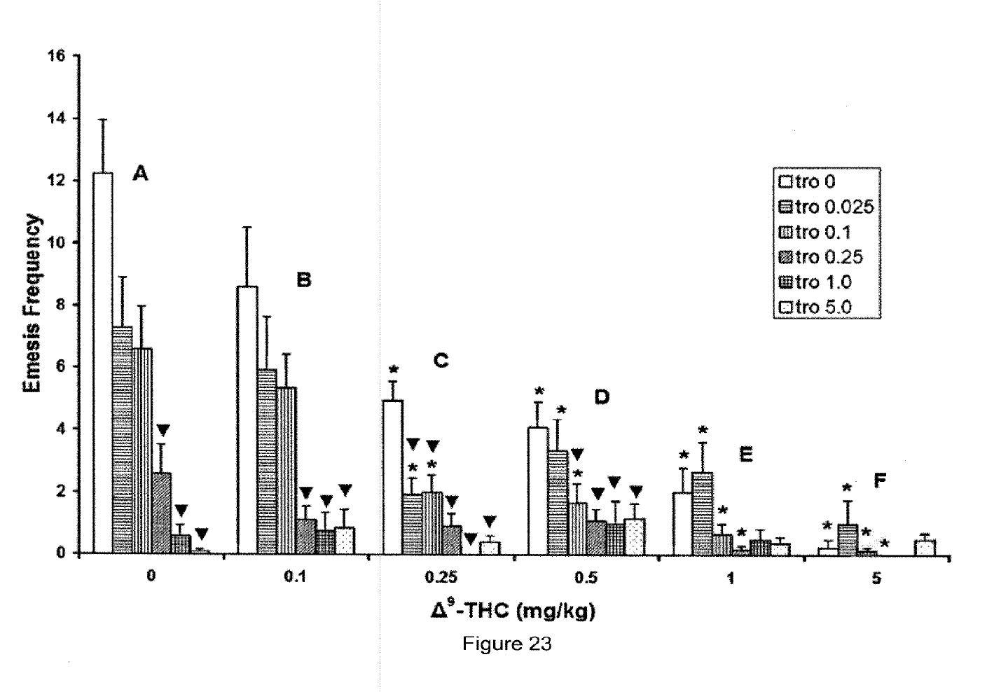

When administered without Δ⁹-THC (FIG. 23, group A), intraperitoneal administration of tropisetron dose-dependently reduced the frequency of CIS-induced emesis relative to its corresponding 0 mg/kg control group, with statistically significant reductions occurring at the 0.25 mg/kg (79% reduction), 1 mg/kg (94%), and 5 (99.8%) mg/kg doses. When pretreated with 0.1 mg/kg Δ⁹-THC (FIG. 23, group B), the 0.25, 1, and 5 mg/kg doses of tropisetron also caused significant reductions in vomiting frequency (88%, 90.6%, and 89.4%, respectively), relative to their corresponding 0 mg/kg control group. At the 0.25 mg/kg Δ⁹-THC dose, significant reductions in vomit frequency occurred at all tested doses of tropisetron (60%, 59.8%, 84%, 100%, and 93%, respectively) relative to their corresponding control group (FIG. 23, group C). At the 0.5 mg/kg Δ⁹-THC dose, tropisetron produced significant reductions in the frequency of emesis at the 0.1 mg/kg (57.5% reduction), 0.25 mg/kg (70%), 1 mg/kg (70.1%), and 5 mg/kg (69.8%) doses, relative to its 0 mg/kg corresponding control group (FIG. 23, group D). None of the cited doses of tropisetron had a significant effect on the frequency of CIS205 induced vomiting, however (FIG. 23, groups E and F) when combined with the 1 mg/kg or 5 mg/kg doses of Δ⁹-THC.

When administered without tropisetron, Δ⁹-THC dose-dependently attenuated CIS-induced vomiting frequency, relative to its corresponding 0 mg/kg vehicle control, with statistically significant reductions noted at the 0.25 mg/kg (59.3% reduction), 0.5 mg/kg (66.7%), 1 mg/kg (83.7%) and 5 mg/kg (96.7%) doses of Δ⁹-THC (FIG. 23, open bars, groups C-F). When co212 administered with 0.025 mg/kg of tropisetron, Δ⁹-THC caused significant reductions in vomiting frequency at doses of 0.25 mg/kg (72.6% reduction), 0.5 mg/kg (56.1%), 1 mg/kg (61.6%), or 5 mg/kg (86.3%) (FIG. 23, horizontally striped bars, groups C-F). A dose of 0.1 mg/kg tropisetron combined with 0.25 mg/kg-5 mg/kg Δ⁹-THC (FIG. 23, vertically striped bars, groups C-F) also caused significantly reduced vomiting frequency (reductions of 69.7%, 74.6%, 88%, and 97.7%, respectively). When combined with 0.25 mg/kg tropisetron, Δ⁹-THC significantly reduced vomiting frequency only at doses of 1 mg/kg (91% reduction) and 5 mg/kg (100%) (FIG. 23, diagonally striped bar, groups E and F). When combined with doses of 1 mg/kg or 5 mg/kg tropisetron, Δ⁹-THC failed to produce significant reductions in the frequency of emesis (FIG. 23, crosshatched and stippled bars). Of the doses of tropisetron and Δ⁹-THC tested in combination, only low doses of Δ⁹-THC (0.25 mg/kg and 0.5 mg/kg) combined with low doses of tropisetron (0.025 mg/kg and 0.1 mg/kg) had significantly greater additive efficacy in reducing the frequency of emesis (FIG. 23 groups C and D).

### Effects of Tropisetron and Δ⁹-THC Alone or in Combination on the Percentage of Animals Vomiting in Response to CIS.

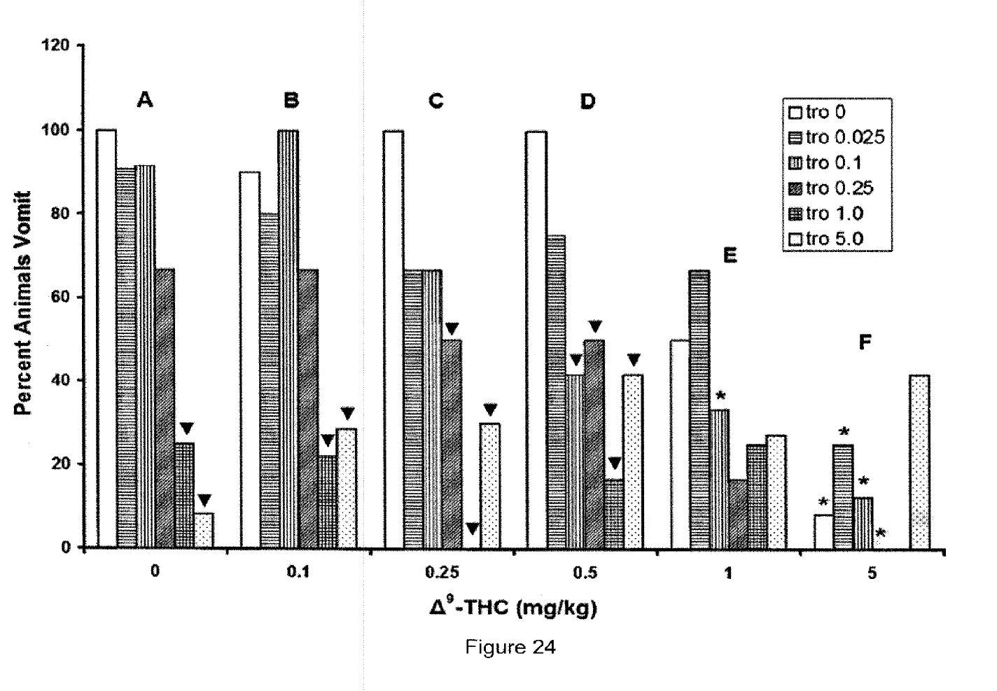

In addition to attenuating emetic frequency, tropisetron administered alone dose-dependently reduced the percentage of shrews vomiting in response to CIS, relative to its 0 mg/kg control group, with statistically significant reductions induced at the 1 mg/kg (75% reduction) and 5 mg/kg (92%) doses (FIG. 24, group A). Similarly, when combined with the 0.1 mg/kg dose of Δ⁹-THC, tropisetron significantly reduced the percentage of shrews vomiting at 1 mg/kg (78%) and 5 mg/kg (71%) doses FIG. 24, group B). When co-administered with 0.25 mg/kg of Δ⁹-THC (FIG. 24, group C), tropisetron significantly reduced the percentage of vomiting shrews at doses of 0.25 mg/kg (50%), 1 (100%), and 5 mg/kg (70%). When co-administered with 0.5 mg/kg of Δ⁹-THC, tropisetron significantly reduced the number of animals vomiting at the 0.1 mg/kg 58% reduction), 0.25 mg/kg (50%), 1 mg/kg (83%), and 5 mg/kg (58%) doses (FIG. 24, group D). In combination with the 1 mg/kg and 5 mg/kg doses of Δ⁹-THC, however, no dose of tropisetron significantly altered the percentage of shrews vomiting (FIG. 24, groups E and F).

Post hoc analysis of the effects of Δ⁹-THC on the percentage of shrews vomiting revealed that when pretreated with the tested doses of tropisetron, the 5 mg/kg dose of Δ⁹-THC had a statistically significant effect when combined with the 0 mg/kg (92% reduction), 0.025 mg/kg (75%), 0.1 mg/kg (88%), and 0.25 mg/kg (100%) doses of tropisetron (FIG. 24, group F, asterisks), relative to the 0 mg/kg Δ⁹-THC control groups. Furthermore, when combined with a dose of 0.1 mg/kg tropisetron, 1 mg/kg Δ⁹-THC significantly reduced the number of shrews vomiting (77%), relative to its corresponding vehicle-injected control group, (FIG. 24, group E, asterisk).

When based on the percentage of animals vomiting in response to CIS, no additive or synergistic antiemetic effects resulted from the tested combinations of Δ⁹-THC and tropisetron (FIG. 24).

### Effects of Dexamethasone and Δ⁹-THC Alone or in Combination on the Frequency of CIS-Induced Emesis.

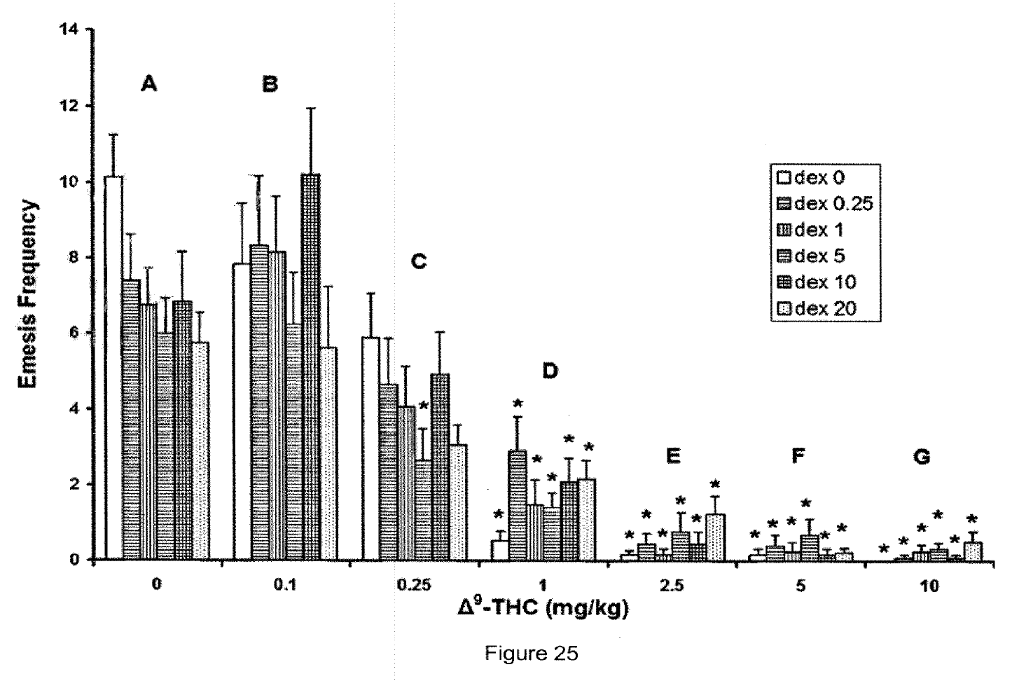

When administered without Δ⁹-THC, no tested doses of dexamethasone significantly attenuated the frequency of CIS-induced vomiting. Although not statistically significant, there appears to be a trend towards a modest reduction in the frequency of emesis (0.05<p<0.1, FIG. 25, group A).

Δ⁹-THC administered alone replicated the results noted in FIG. 23, in that it dose-dependently reduced the frequency of emesis produced by CIS relative to its corresponding 0 mg/kg (vehicle) control group (FIG. 25, groups D-G). When combined with DEX at doses of 1, 5, 10, or 20 mg/kg, Δ⁹-THC caused significant reductions (60.8%-100%) in vomiting frequency at all tested doses (FIG. 25, groups D-G, asterisks). Additionally, coadministration of 5 mg/kg DEX with the 0.25 mg/kg dose of Δ⁹-THC also significantly reduced (55.5%) the frequency of vomiting (FIG. 25, group C).

### Effects of Dexamethasone and Δ⁹-THC Alone or in Combination on the Percentage of Animals Vomiting in Response to CIS.

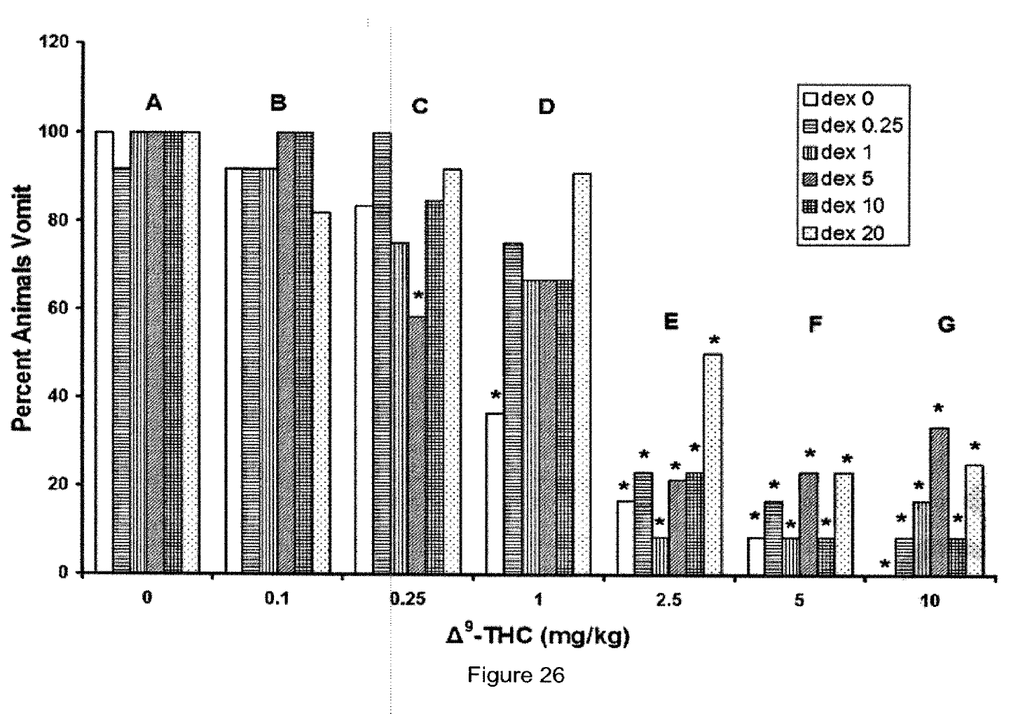

Based on analysis with Fisher's exact test, no dose of DEX administered alone significantly reduced the number of shrews vomiting in response to CIS (FIG. 26, group A). Furthermore, even when combined with any of the tested doses of Δ⁹-THC, no dose of DEX was able to significantly reduce the number of shrews vomiting relative to the 0 mg/kg DEX control groups (FIG. 26).

The tested doses of Δ⁹-THC did, however, reduce the number of shrews expressing CIS-induced vomiting in an essentially dose288 dependent manner. When administered without DEX, Δ⁹-THC at 1 mg/kg (FIG. 26, group D) or greater (FIG. 26, open bars, groups E-G), induced significant reductions (64%, 83.3%, 91.6%, and 100%, respectively) in the percentage of shrews vomiting, relative to their vehicle-injected control group (FIG. 26, group A). The 2.5 mg/kg dose of Δ⁹-THC (FIG. 26, group E) significantly reduced the percentage of shrews vomiting when co-administered with any of the tested doses of DEX (83%, 77%, 92%, 79%, 77%, and 50%, respectively), relative to the 0 mg/kg Δ⁹-THC control group. In addition, coadministration of 5 mg/kg of Δ⁹-THC and DEX (FIG. 26, group F) significantly reduced the percentage of animals vomiting (92%, 83%, 92%, 77%, 92%, and 77%, respectively). The 10 mg/kg dose of Δ⁹-THC (FIG. 26, group G) also significantly reduced the percentage of shrews vomiting whether administered without (100% reduction) or with DEX (92%, 83%, 67%, 92%, and 75%, respectively).

Finally, in shrews pretreated with 5 mg/kg DEX, the 0.25 mg/kg dose of Δ⁹-THC induced a 42% reduction in the percentage of shrews vomiting relative to its vehicle-injected control group (FIG. 26, diagonally striped bar, group C). Overall, at the doses tested, Δ⁹-THC had no significant additive or synergistic antiemetic activity when combined with DEX.

### Example 5

### Δ⁹-THC Suppresses Vomiting Behavior and Fos Expression in both Acute Phase and Delayed Phase of CIV

### Animals

Adult female Least Shrews (C. parva, N=44) from the Western University Animal Facilities colony were housed in groups on a 14:10 light:dark cycle and fed and watered ad libitum. The shrews were 45-60 days old and weighed 3.7-5.4 g. All experiments were performed between 11:00 hr and 17:30 hr, and in accordance with Western University IACUC standards. On the day of experimentation, shrews were brought from the animal facility and separated to individual cages, and allowed to adapt to the new conditions for 2 hr to 3 hr minimum to reduce potential novelty-induced Fos immunoreactivity.

Behavioral Procedures

For the acute phase studies, shrews were placed individually in experiment cages and allowed 2 hr to adapt, during which time food was restricted. They were then given four mealworms (Tenebrio sp.) each, and THC (2.5 mg/kg, i.p., provided through NIDA, Rockville, Md.) or vehicle (ethanol:Emulphor™:saline in a 1:1:18 ratio) injection. Twenty minutes after THC injection, shrews were injected with 10 mg/kg CIS, i.p. (Sigma-Aldrich, St. Louis, Mo.) then observed for vomiting behavior for 30 min. For these studies, a shrew was transcardially perfused 75 min after vomiting occurred, typically 20 min-30 min after CIS injection. Thus, vomiting animals were perfused 95 min-105 min after CIS injection. Animals used in the acute phase studies that did not vomit were perfused 90 min-100 min after CIS injection.

To begin the delayed phase studies, the shrews were injected with CIS as described above, then placed back in their home cage with food and water ad lib overnight. The next morning the shrews were moved individually to experiment cages and given adaptation time and food restriction as in the acute studies. At 31 hr post-CIS injection, shrews were given four mealworms, followed 20 min later by i.p. injection of THC or vehicle, and then monitored for vomiting for 30 min. For the delayed phase studies, shrews were perfused 90 min-100 min after THC injection if there was no vomiting, or 65 min-75 min after vomiting (95 min-105 min post-THC injection). In all cases, injections for a given day were staggered by 45 min each, to ensure perfusions would occur during optimal emesis-related Fos expression.

Lastly, two groups of shrews received an i.p. injection of the CB₁ receptor antagonist SR141716A (2.0 mg/kg, Sanofi, France) dissolved in the same vehicle as THC, or a vehicle injection, 20 min before receiving a THC injection. CIS injection and monitoring for vomiting behavior, as well as perfusion timing, were then performed as described above.

### Immunohistochemical Procedures

After the appropriate time period for acute or delayed phase vomiting, shrews were anesthetized with a lethal dose of pentobarbital (100 mg/kg) and perfused transcardially via blunted needle with a peristaltic pump. The shrew was perfused with ice cold 4% paraformaldehyde and 5% picric acid in pH 7.4, 0.1 M phosphate buffer (10 min). Brains were removed and stored in 30% sucrose in 0.1 M PB overnight, then embedded in blocks of 12% gelatin in 30% sucrose/PB. The blocks were postfixed for 3 hr in 2% paraformaldehyde/PB, then rinsed and immersed in 30% sucrose/PB until they sank (usually 1-2 hr). The brain block was cut sagittally on a freezing benchtop microtome (Leica) at 30 μm into 5 series, and stored in PB with 0.03% sodium azide.

Immunolabeling was accomplished by blocking a free-floating series with 10% normal horse serum (NHS) and 3% hydrogen peroxide in PB with 0.3% Tri-ton X-100 (TX) for 30 min. After rinsing in PB, tissue was put in sheep anti-Fos polyclonal antibody (Chemicon/Millipore, Temecula, Calif.; 1:600), with 5% NHS and 0.3% TX in PB, and incubated for about 42 hr at room temperature (RT) with gentle shaking. After rinsing in PB, the tissue was placed in biotinylated donkey anti-sheep IgG secondary antibody (Jackson Immunoresearch, West Grove, Pa.; 1:600) diluted in the same diluent described for the primary antibody, and the tissue incubated at RT with shaking for 75 min Tissue was then rinsed and incubated for 60 min in HRP-conjugated avidin-biotin complex (Vector Labs, Burlingame, Calif.; Vectastain kit diluted 1:2) in PB. Tissue was then rinsed twice in PB, then once in imidazole-acetate buffer (0.1 M, pH 7.4), then reacted for 6 min in 0.0006% hydrogen peroxide with 2% nickel ammonium sulfate-enhanced diaminobenzidine (DAB, 0.05% in 0.1 M imidazole-acetate buffer). All reagents for DAB processing were purchased from Sigma-Aldrich. Other series were processed for immunofluorescence using one or more primary antibodies including: mono-clonal rat anti-Substance P (Chemicon/Millipore, 1:400), affinity-purified polyclonal rabbit anti-CB₁ receptor (Chemicon/Millipore, AB5636P, 1:333), affinity-purified polyclonal rabbit anti-CB₂ receptor (Chemicon/Millipore, AB5642P, 1:250), and polyclonal rabbit anti-serotonin (Invitrogen/Zymed, 1:400). The cannabinoid receptor antibodies were both affinity-purified stocks with accompanying Western Blot data and characterization references provided by the manufacturer. In addition, several reactions were performed as described, but with normal donkey serum in place of the primary antibody (zero-primary controls). Tissue was blocked, and primary antibodies reacted overnight after rinsing as described for the anti-Fos primary. Secondary antibodies were HRP-conjugated donkey IgG (Jackson Immunoresearch) raised against the appropriate species (rat or rabbit), diluted 1:600 in the same diluent described above. Series were reacted for 90 min, then rinsed three times in PB, then reacted in the dark with AlexaFluor-405, 488, or 594 conjugated tyramide produced in lab using reactive AlexaFluor™ dyes from Invitrogen and tyramine from Sigma-Aldrich. Purified tyramide conjugate was reacted for 20 min with 0.006% hydrogen peroxide in PB.

After reacting, tissue was rinsed thoroughly in PB and mounted onto gel-subbed slides out of PB. After air-drying, slides were dehydrated through a series of ascending ethanols (50%-75%-90%-100%), then cleared in xylene. Cleared slides were cover-slipped with DEPEX (Electron Microscopy Sciences, Hatfield, Pa.).

### Analysis

Photomicrographs of regions of interest were taken at 1600×1200 px digital resolution with a SPOT digital camera (Diagnostic Instruments) attached to a PC running version 4.0 of the SPOT software and mounted to a Nikon Eclipse E600 microscope. Images were exported to Adobe Photoshop 7, and passed through a high-pass threshold filter set to 75% black. This filter eliminates potential false positives created by nonspecific background labeling. Relevant structures were identified using an atlas produced in lab (Ray & Darmani, 1156 Brain Res. 99-111 (2007). Immunofluorescent series were examined qualitatively for each primary antibody using a Nikon three-laser scanning confocal microscope, noting the relative density of the various markers in the DVC, and noting terminals or other structures which were co-labeled for several different antigens.

### Statistics

The number of shrews vomiting in each treatment group was recorded and the results analyzed using Mann-Whitney U tests for unpaired samples, with a significance level of P≦0.05 being considered statistically significant differences between groups. The number of animals vomiting in the acute phase vs. delayed phase was also analyzed via Mann-Whitney U tests. After immunohistochemical processing for Fos-IR, cell counts for each structure were averaged, and two-way analysis of variance and Student's t-test were used to compare numbers of Fos+nuclei.

### Behavioral Effects of THC and SR141716A on CIS-Induced Vomiting

Regarding the behavioral effects of THC and SR141716A on CIS-induced vomiting The number of shrews vomiting is described in Table 2. In generating that data, shrews were injected with CIS (10 mg/kg, i.p.), then injected at the appropriate time with either vehicle, Δ⁹-THC (2.5 mg/kg, i.p.), or Δ⁹-THC and the CB₁ receptor antagonist SR141716A (2 mg/kg, i.p.). Groups were divided according to pretreatment condition, treatment condition and emetic phase (acute or delayed). No shrews vomited when vehicle was injected in place of CIS. The number of shrews vomiting in response to CIS injection when also treated with Δ⁹-THC was significantly less than those given CIS followed by either vehicle injection (*P<0.05; **P<0.01), or by both SR141716A and Δ⁹-THC injection (†p<0.05; ††p<0.01). This pattern held true for both acute and delayed phases. There were no significant differences in the number of animals vomiting when a given pretreatment/treatment combination was compared between acute and delayed phase conditions.

TABLE 2
Number of shrews vomiting per experimental group

| | Treatment | Vomiting | Number vomited |
|---|---|---|---|
| Pretreatment condition | condition | phase | per total in group |
| Vehicle | Vehicle | Acute | 0/6 |
| Vehicle | Cisplatin | Acute | 6/6 |
| Δ⁹-THC | Cisplatin | Acute | 1/8 |
| Δ⁹-THC + SR141716A | Cisplatin | Acute | 5/6 |
| Vehicle | Vehicle | Delayed | 0/6 |
| Vehicle | Cisplatin | Delayed | 5/6 |
| Δ⁹-THC | Cisplatin | Delayed | 2/9 |
| Δ⁹-THC + SR141716A | Cisplatin | Delayed | 6/9 |

No shrews vomited when injected with vehicle instead of CIS. When administered following CIS, THC injections blocked vomiting in significantly more shrews than vehicle injections in both the acute (Mann-Whitney U score=45, 3; P<0.005) and delayed (U=43.5, 10.5; P<0.02) phases. Administration of the CB₁ receptor-specific antagonist SR141716A prior to THC injection significantly reversed the antiemetic efficacy of THC in both acute (U=41, 7; P<0.01) and delayed (U=58.5, 22.5; P<0.05) phases, resulting in vomiting in a number of shrews comparable to the vehicle-injected treatment groups in both phases (acute phase, p>0.24; delayed phase, p>0.27). No significant differences were found in the numbers of shrews vomiting in the acute phase vs. the delayed phase in either the vehicle groups (P>0.24), the THC groups (P>0.33), or the SR141716A+THC groups (P>0.27).

Effects of THC and SR141716A on Fos Expression Resulting from CIS-Induced Vomiting

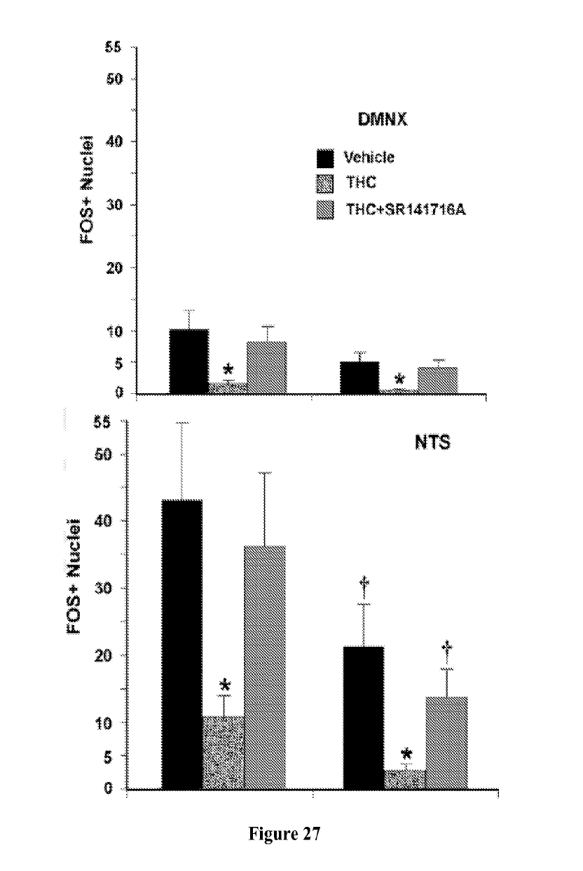

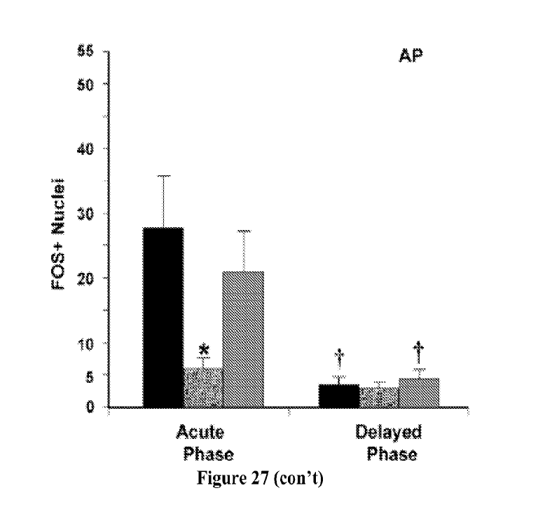

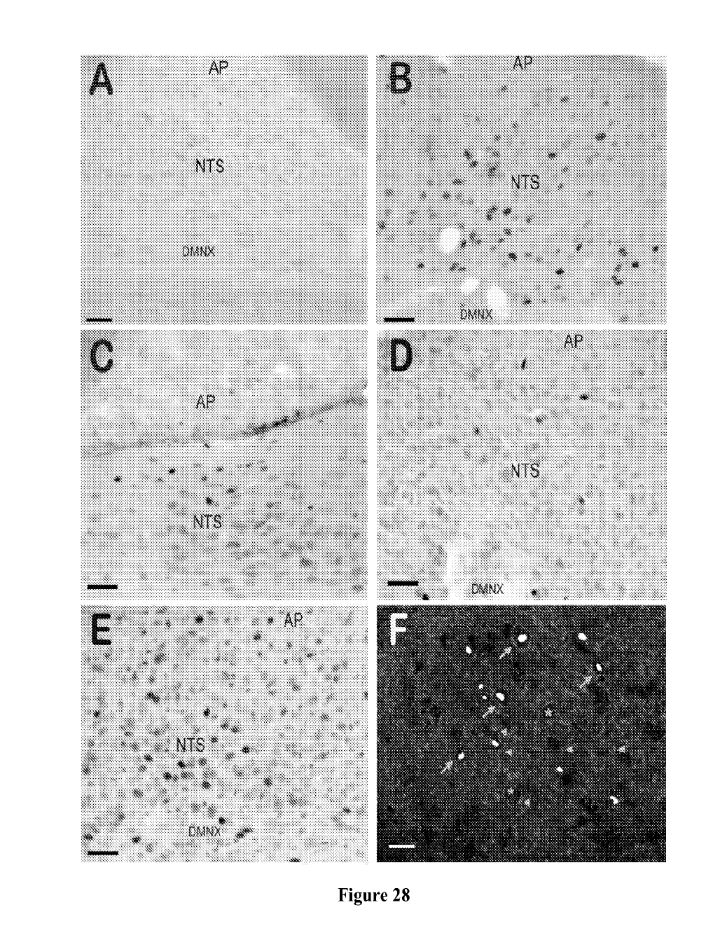

The number of Fos-immunopositive nuclei in the subdivisions of the DVC following CIS injections in each group is presented in FIG. 27, and representative photomicrographs of Fos-IR are presented in FIG. 28. Vehicle injected in place of CIS (FIG. 28A) produced almost no Fos-IR in the DVC, while CIS-induced vomiting resulted in numerous Fos-IR nuclei (FIG. 28B). In the acute phase of CIV, THC injections (FIG. 28D) significantly (P<0.05) reduced the number of Fos-immunopositive nuclei compared to vehicle injections (FIG. 28B). This was the case for all three nuclei within the DVC (NTS, AP, and DMNX). When SR141716A was administered prior to THC (FIG. 28E), the number of Fos+nuclei was not significantly different (P>0.2) from vehicle injections, and was significantly higher (p<0.05) than when THC was administered alone. In the delayed phase (FIG. 28C), THC-injected animals had significantly fewer (P<0.05) Fos-immunopositive nuclei than vehicle-injected animals in the NTS and DMNX, but not the AP (P>0.33). The difference in Fos+nuclei between vehicle-injected and SR141716A+THC-injected groups was not significant for any part of the DVC (P>0.18).

When regions of the DVC with the same treatment conditions were compared between acute and delayed phases, there were significantly fewer Fos+nuclei in the DVC during the delayed phase vs. the acute phase. This effect was region and condition-specific, however. The AP and NTS had significantly fewer (P<0.05) Fos+nuclei per section in the delayed phase than in the acute phase, but only when treated with vehicle or SR141716A+ THC. The number of Fos+nuclei was not significantly different when treated with THC alone (P>0.1). In the DMNX, no significant differences (P>0.12) were found between the delayed phase and the acute phase.

### Immunohistochemical Labeling of Neurochemicals in the Dorsal Vagal Complex

Close examination of overlaid Fos−/CB₁-IR images of the NTS demonstrated Fos+nuclei amidst dense CB₁-IR terminal-like structures, including several neurons which appeared apposed to CB₁-IR structures (FIG. 28F). CB₁-IR was more prominent in the NTS and DMNX than in the AP (FIG. 29A). CB₂-IR was found on the surface of the brainstem and in the choroid plexus, as well as in ribbon-like structures traversing dorsoventrally through the brainstem (FIG. 29B). These structures lacked a soma or fine fibers indicative of neurons, suggesting non-neuronal elements such as glia or brain vasculature were being labeled. Additional labeling for 5-HT and SP-IR demonstrated numerous fibers and putative terminals for both neurotransmitters within the DVC. Tissue labeled concurrently for SP, 5-HT, and CB₁ receptor-IR showed a wide variety of immunolabeled terminal-like structures, including single-labeled, double-labeled, and triple-labeled structures (FIG. 29C). Only Fos immunolabeling was quantified, however. When the primary anti-bodies were left out, no specific labeling was found for either the anti-rat (FIG. 29D) or anti-rabbit (FIG. 29E) secondary antibodies. These controls also demonstrated that background fluorescence was much dimmer than specific fluorescence using either Alexa594- or Alexa488-conjugated tyramides.

1. The use of Cryptotis parva as a specific and rapid behavioral model to screen concomitantly both the CNS penetration and the antiemetic potential of tachykinin NK₁ receptor antagonists.

2. A method of testing the anti-emetic efficacy of a tachykinin NK₁ receptor antagonist as a therapeutic agent for treating emesis comprising:
(a) obtaining test animals of an animal model of emesis, Cryptotis parva, which animal exhibits CNS penetration and emesis in response to tachykinin NK₁ receptor agonists;
(b) dividing test animals into two groups;
(c) administering a potential tachykinin NK₁ receptor antagonist to one group of animals and its vehicles to the other group;
(d) administering a tachykinin NK₁ receptor agonist to both groups of animals;
(e) observing and comparing indices of CNS penetration and emesis in said groups.

3. The use of Cryptotis parva as a specific and rapid behavioral model to screen concomitantly both the emetic capacity of diverse leukotrienes and the antiemetic potential of at least one leukotriene antagonist.

4. The use of C. parva of claim 4, wherein said leukotriene antagonist is a CysLT₁ receptor antagonist or an inhibitor of leukotriene synthetic enzymes.

5. A method of testing the antiemetic efficacy of a CysLT₁ receptor antagonist or an inhibitor of leukotriene synthetic enzymes as therapeutic agents for treating emesis comprising:
(a) obtaining test animals of an animal model of emesis, Cryptotis parva, which animal exhibits emesis in response to leukotriene LTC₄ or LTD₄;
(b) dividing test animals into at least two groups;
(c) administering a potential CysLT₁ receptor antagonist to one group of animals to pretreat said animals;
(d) administering a leukotriene LTC₄ (or LTD₄) to a non-pretreated group of animals and to the CysLT₁ receptor antagonist-pretreated group of animals;
(e) observing and comparing indices emesis in said groups;
(f) optionally repeating steps (b)-(e) against other emetogens;
(g) optionally administering a potential CysLT₁ receptor antagonist either alone or in combination with one or more of other classes of antiemetics such as a 5-HT₃ receptor antagonist, an NK₁ receptor antagonist, or an anti-inflammatory agent such as dexamethasone to determine their antiemetic potential and possible additive and/or synergistic antiemetic activity against both phases of chemotherapy-induced vomiting; and
(h) optionally administering a potential leukotriene synthesis inhibitor either alone or in combination with one or more of other classes of antiemetics to determine their antiemetic potential and possible additive and/or synergistic antiemetic activity against both phases of chemotherapy-induced vomiting.

6. The method of claim 5, wherein said other emetogens in step (f) are bacterial toxins or inflammatory emetogens

7. The method of claim 5, wherein the other class antiemetics includes a 5-HT₃ receptor antagonist, an NK₁ receptor antagonist, or an anti-inflammatory agent.

8. The method of claim 7, wherein the anti-inflammatory agent is dexamethasone.

9. The method of claim 3, further comprising the step of including a bacterial toxin or toxoid as an additional inflammatory emetogen.

10. A medicament for the treatment of emesis comprising a leukotriene antagonist.

11. The medicament of claim 10, wherein said leukotriene antagonist is a cysteinyl (peptidyl) leukotriene receptor antagonist (LTRA).

12. The medicament of claim 11, wherein said LTRA is pranlucast.

13. The use of Cryptotis parva as a specific and rapid behavioral model to screen for examining the substrates of cannabinoid-mediated inhibition of both the immediate and delayed phases of chemotherapy-induced vomiting comprising immunolabeling for at least one of serotonin, Substance P, cannabinoid receptors 1 or 2, or the neuronal activation marker Fos.

14. The use of Cryptotis parva as a specific and rapid behavioral model to screen for synergy between two or more antiemetic agents effective in one or both the immediate and delayed phases of chemotherapy-induced vomiting.

15. The use of the model of claim 14 wherein the antiemetic agents are cannabanoids, 5-HT₃ antagonists, leukotriene antagonists, or corticosteroids.
